(19) United States
(12) Patent Application Publication
(10) Pub. No.: US 2024/0327489 A1
(43) Pub. Date: Oct. 3, 2024

(54) BUTYROPHILIN-LIKE 2 AGENTS FOR TREATING INFLAMMATORY DISORDERS

(71) Applicant: Regeneron Pharmaceuticals, Inc., Tarrytown, NY (US)

(72) Inventors: Casandra M. Panea, West New York, NJ (US); Sokol Haxhinasto, Brookfield, CT (US); Zaruhi Hovhannisyan, Hoboken, NJ (US); Julie Horowitz, White Plains, NY (US)

(21) Appl. No.: 18/580,770

(22) PCT Filed: Jul. 20, 2022
(86) PCT No.: PCT/US2022/073936

§ 371 Date: Jan. 19, 2024

Related U.S. Application Data
(60) Provisional application No. 63/223,644, filed on Jul. 20, 2021.

(51) Int. Cl. C07K 14/705; A61K 38/00; A61K 39/00; A61P 29/00; A61P 37/06; C07K 16/28; G01N 33/50

(52) U.S. Cl. CPC C07K 14/70503; A61P 29/00; A61P 37/06; C07K 16/2803; G01N 33/505; A61K 38/00; A61K 2039/505; C07K 2319/33

(57) ABSTRACT

This disclosure provides methods for treating T cell-driven inflammatory disorders. The methods include administering to a subject a therapeutically effective amount of a butyrophilin-like 2 (Btnl2) agent capable of increasing a level or activity of Btnl2, thereby altering the frequencies or function of lymphocytes in the subject.

### FIELD

The present disclosure relates generally to methods and reagents for treating inflammatory disorders, including to methods and reagents for treating inflammatory disorders via Btnl2-mediated targeting of T cell development, maintenance, and function.

### BACKGROUND

Inflammation is a complex cellular and biochemical process that occurs in the affected blood vessels and adjacent tissues in response to an injury or abnormal stimulation caused by a physical, chemical, or biologic agent, such as a pathogen, allergen or irritant. Inflammatory bowel disease (IBD) is a group of inflammatory conditions of the colon and small intestine that cause over 50,000 deaths annually. The causes of IBD are complex, and contributing factors may include diet, genetics, and the composition of an individual's gut microflora. Medical treatment is largely based on factors specific to an individual.

Tissue-resident intraepithelial lymphocytes (IELs) represent a heterogenous population of antigen-experienced immune cells in the intestinal epithelium that are involved in the maintenance of gut homeostasis. In particular, IELs expressing alpha-beta (αβ) T cell receptors (TCRs) are poised for mounting pathogen-specific memory responses, while those possessing gamma-delta (γδ) TCRs strengthen tight junctions and orchestrate innate and adaptive immunity during homeostasis, inflammation, and infection. Interactions between intestinal epithelial cells (IECs) and γδ IELs influence IEL development and function. Notably, recent studies emphasized that anatomical segregation could drive gut segment-specific immunity, including functionally distinct γδ IEL immune responses to chemically-induced and pathogen-induced epithelial injury. However, the mechanisms that regulate γδ IEL development and maintenance in response to the local antigenic environment remain poorly understood.

Recent studies provided some evidence that IEC-specific butyrophilin-like (Btnl) molecules induce perinatal expansion and maturation of distinct Vγ TCR⁺ IELs. Indeed, intestinal γδ IELs predominantly express Vγ7 in mice and Vγ4 in humans that persist throughout the life of the host. γδ IELs continuously sample both self and bacterial antigens from the local environment to customize their TCR specificities. Moreover, the contributions of Btnl molecules to shaping γδ TCR repertoire diversity and regulating the distribution and function of γδ IEL subsets across intestinal compartments remain to be elucidated and may inform the understanding of the compartmentalized immune responses observed in the intestine.

Btn/Btnl proteins are members of B7 immunoglobulin-superfamily and analogous to other costimulatory and coinhibitory molecules (e.g., CD80, CD86, PDL1, and PDL2) have been shown to modulate αβ T cell immune functions, including inhibition of CD4+T and CD8*T cell activation, proliferation and cytokine production, induction of regulatory T (Treg) cells and blockade of antigen-specific proinflammatory responses. Btnl2, a member of the Btnl family, has been shown to induce Treg differentiation and suppress T cell activation and proliferation in vitro. However, the role of Btnl2 in regulating intestinal immune responses during homeostasis and inflammation and, particularly, in the induction and maintenance of intestinal γδ IELs has not yet been addressed.

### SUMMARY

In one aspect, this disclosure provides a method of treating an inflammatory disorder, comprising (a) selecting a subject with an inflammatory disorder and an elevated level of intraepithelial lymphocytes (IELs) as compared to a predetermined reference value; and (b) administering to the subject a therapeutically effective amount of a butyrophilin-like 2 (Btnl2) agent that is capable of decreasing proliferation of IELs in the subject by increasing a level or activity of Btnl2 in the subject.

In some embodiments, the subject has at least one loss-of-function single nucleic polymorphism (SNP) at rs28362675 in the Btnl2 gene. In some embodiments, the subject has at least one loss-of-function SNP at rs28362675 that results in a Glu454Ter (stop codon) mutation.

In some embodiments, the IELs comprise γδ T cells. In some embodiments, the IELs comprise intestinal γδ IELs. In some embodiments, the intestinal γδ IELs comprise jejunal γδ IELs, ileal γδ IELs, colonic γδ IELs, or combinations thereof. In some embodiments, the intestinal γδ IELs comprise the ileal γδ IELs. In some embodiments, the intestinal γδ IELs comprise ileal CD8αα+γδ IELs.

In some embodiments, the inflammatory disorder is an intestinal inflammatory disorder. In some embodiments, the inflammatory disorder is an immune-mediated disease. In some embodiments, the inflammatory disorder is an autoimmune disease. In some embodiments, the inflammatory disorder is a gut-associated immune-mediated disease. In some embodiments, the immune-mediated disease comprises graft versus host disease (GVHD) (with or without intestinal manifestations), Celiac disease, ulcerative colitis, Crohn's disease, rheumatoid arthritis, sarcoidosis, myositis, inflammatory bowel disease, gastrointestinal inflammation, or type I diabetes.

In some embodiments, the Btnl2 agent comprises a Btnl2 protein or variant thereof or a fusion protein comprising the Btnl2 protein or variant thereof. In some embodiments, the Btnl2 protein comprises an amino acid sequence having at least 90% identity to an amino acid sequence of SEQ ID NOs: 1-3 or comprises an amino acid sequence of SEQ ID NOs: 1-3.

In some embodiments, the fusion protein further comprises a tissue-specific signal. In some embodiments, the tissue-specific signal comprises a MADCAM1 inhibitor or an anti-MADCAM1 antibody. In some embodiments, the fusion protein comprises the Btnl2 protein or variant thereof and an anti-MADCAM1 antibody.

In some embodiments, the Btnl2 agent comprises a vector having a polynucleotide sequence encoding the Btnl2 protein or variant thereof or encoding a fusion protein comprising the Btnl2 protein or variant thereof. In some embodiments, the Btnl2 agent comprises a nucleic acid having a polynucleotide sequence encoding the Btnl2 protein or variant thereof or encoding a fusion protein comprising the Btnl2 protein or variant thereof. In some embodiments, the polynucleotide sequence is RNA.

In some embodiments, the subject is a mammal. In some embodiments, the subject is human. In some embodiments, the Btnl2 agent is administered intratumorally, intravenously, subcutaneously, intraosseously, orally, transdermally, or sublingually. In some embodiments, the Btnl2 agent is administered in a sustained release, controlled release, or delayed release dosage form.

In some embodiments, the method further comprises administering to the subject a second therapeutic agent or therapy. In some embodiments, the second therapeutic agent comprises an anti-inflammation agent.

In another aspect, this disclosure provides a method of treating an inflammatory bowel disease (IBD), comprising: (a) selecting a subject having an IBD and at least one loss-of-function SNP at rs28362675 in the Btnl2 gene; and (b) administering to the subject a therapeutically effective amount of a Btnl2 agent capable of increasing a level or activity of Btnl2 in the subject.

In some embodiments, the subject has at least one loss-of-function SNP at rs28362675 that results in a Glu454Ter (stop codon) mutation. In some embodiments, the Btnl2 agent comprises a Btnl2 protein or variant thereof or a fusion protein comprising the Btnl2 protein or variant thereof. In some embodiments, the Btnl2 protein comprises an amino acid sequence having at least 90% identity to an amino acid sequence of SEQ ID NOs: 1-3 or comprises an amino acid sequence of SEQ ID NOs: 1-3.

In some embodiments, the Btnl2 agent comprises a vector having a polynucleotide sequence encoding the Btnl2 protein or variant thereof or a fusion protein comprising the Btnl2 protein or variant thereof. In some embodiments, the Btnl2 agent comprises a nucleic acid having a polynucleotide sequence encoding the Btnl2 protein or variant thereof or a fusion protein comprising the Btnl2 protein or variant thereof. In some embodiments, the polynucleotide sequence is RNA.

In some embodiments, the method comprises the step of selecting comprises obtaining a sample containing a nucleic acid from the subject and performing a genotyping assay on the sample.

In another aspect, this disclosure also provides a method of decreasing the number of IELs in a subject, comprising administering an effective amount of a Btnl2 agent to a subject in need thereof, the Btnl2 agent capable of increasing a level or activity of Btnl2 in the subject. In some embodiments, the IELs comprise γδ T cells. In some embodiments, the IELs comprise intestinal γδ IELs. In some embodiments, the intestinal γδ IELs comprise jejunal γδ IELs, ileal γδ IELs, colonic γδ IELs, or combinations thereof. In some embodiments, the intestinal γδ IELs comprise the ileal γδ IELs. In some embodiments, the intestinal γδ IELs comprise ileal CD8αα+γδ IELs.

In some embodiments, the Btnl2 agent comprises a Btnl2 protein or variant thereof or a fusion protein comprising the Btnl2 protein or variant thereof. In some embodiments, the Btnl2 protein comprises an amino acid sequence having at least 90% identity to an amino acid sequence of SEQ ID NOs: 1-3 or comprises an amino acid sequence of SEQ ID NOs: 1-3.

In some embodiments, the fusion protein further comprises a tissue-specific signal. In some embodiments, the tissue-specific signal comprises a MADCAM1 inhibitor or an anti-MADCAM1 antibody. In some embodiments, the fusion protein comprises the Btnl2 protein or variant thereof and an anti-MADCAM1 antibody.

In some embodiments, the Btnl2 agent comprises a vector having a polynucleotide sequence encoding the Btnl2 protein or variant thereof or encoding a fusion protein comprising the Btnl2 protein or variant thereof. In some embodiments, the Btnl2 agent comprises a nucleic acid having a polynucleotide sequence encoding the Btnl2 protein or variant thereof or encoding a fusion protein comprising the Btnl2 protein or variant thereof. In some embodiments, the polynucleotide sequence is RNA.

In another aspect, this disclosure further provides a method of eliciting an antibacterial response in a subject, comprising administering to the subject an effective amount of a Btnl2 agent, the Btnl2 agent capable of increasing a level or activity of Btnl2 in the subject.

In some embodiments, the IELs comprise γδ T cells. In some embodiments, the IELs comprise intestinal γδ intraepithelial lymphocytes (IELs). In some embodiments, the intestinal γδ IELs comprise jejunal γδ IELs, ileal γδ IELs, colonic γδ IELs, or combinations thereof. In some embodiments, the intestinal γδ IELs comprise the ileal γδ IELs. In some embodiments, the intestinal γδ IELs comprise ileal CD8αα+γδ IELs.

In some embodiments, the Btnl2 agent comprises a Btnl2 protein or variant thereof or a fusion protein comprising the Btnl2 protein or variant thereof. In some embodiments, the Btnl2 protein comprises an amino acid sequence having at least 90% identity to an amino acid sequence of SEQ ID NOs: 1-3 or comprises an amino acid sequence of SEQ ID NOs: 1-3.

In some embodiments, the fusion protein further comprises a tissue-specific signal. In some embodiments, the tissue-specific signal comprises a MADCAM1 inhibitor or an anti-MADCAM1 antibody. In some embodiments, the fusion protein comprises the Btnl2 protein or variant thereof and an anti-MADCAM1 antibody.

In some embodiments, the Btnl2 agent comprises a vector having a polynucleotide sequence encoding the Btnl2 protein or variant thereof or encoding a fusion protein comprising the Btnl2 protein or variant thereof. In some embodiments, the Btnl2 agent comprises a nucleic acid having a polynucleotide sequence encoding the Btnl2 protein or variant thereof or encoding a fusion protein comprising the Btnl2 protein or variant thereof. In some embodiments, the polynucleotide sequence is RNA.

In another aspect, this disclosure provides a method for identifying a Btnl2 agent capable of decreasing IELs in a subject, comprising: (a) administering to the subject an amount of a Btnl2 agent capable of increasing a level or activity of Btnl2 or having Btnl2 agonist activity; (b) performing an assay on a sample obtained from the subject and determining the number of the IELs in the sample; and (c) identifying the Btnl2 agent as having capability of decreasing the IELs in the subject if the subject has an increased number of the IELs as compared to a predetermined reference value.

In some embodiments, the IELs comprise intestinal γδ IELs. In some embodiments, the intestinal γδ IELs comprise duodenal γδ IELs, jejunal γδ IELs, ileal γδ IELs, or combinations thereof.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, and 1C show that Btnl2 is preferentially expressed in small intestinal epithelial cells. FIG. 1A shows a schematic representation of the WT and targeted locus of Btnl2−/− mice. hUb, human Ubiquitin promoter. FIG. 1B shows the results of beta-galactosidase and Neutral Red counterstaining in cryosections of segments of the small intestine (duodenum, jejunum, and ileum) and colon of 15-wk-old Btnl2-KO mice. Arrowheads indicate regions of duodenal glands and duodenal, jejunal, ileal, and colonic crypts and villi with weak Btnl2-LacZ expression. Magnification is 20×. FIG. 1C shows mRNA expression of Btnl2 in intestinal epithelial cells from different segments of small intestine and colon of cohoused 7-wk-old Btnl2-KO and WT mice (n=5, each), normalized to β2m. Error bars represent mean±SEM. Significance is measured using multiple unpaired t-tests assuming similar SD, *p<0.05, ** p<0.005, *** p<0.0005, **** p<0.0001, significantly different from WT mice.

FIGS. 2A, 2B, 2C, 2D, and 2E show that Btnl2-KO mice display increased frequencies of γδ IELs in the ileum. FIG. 2A shows that different segments of small intestine were collected from cohoused 7-17-wk-old Btnl2-KO and WT mice (n=3-8, each) and processed for flow cytometry. Left panel: representative flow cytometry plots of γδ IELs in the duodenum, jejunum, and ileum of cohoused 7-wk-old Btnl2-KO and WT littermates. Displayed plots are gated on live TCRαβ-cells. Right panel: frequencies of γδ IELs and CD8αα+γδ IELs from 7-17-wk-old Btnl2-KO and WT littermates. FIG. 2B shows frequencies of ileal γδ IELs at different ages (n=6-23 mice/group). Error bars represent mean±SEM. Significance is measured by 2-way ANOVA with Sidak's multiple comparison test, *p<0.05, ** p<0.005. FIG. 2C shows frequencies of ileal αβ IELs at different ages (n=6-23 mice/group). Error bars represent mean±SEM. Significance is measured by 2-way ANOVA. FIG. 2D shows that ileum was collected from cohoused Btnl2-KO and WT littermates of different ages and intestinal intraepithelial lymphocytes (IELs) were isolated and processed for flow cytometry. Left panel: representative flow cytometry plots of γδ IELs in the ileum of 12-17-wk-old Btnl2-KO and WT littermates. Right panel: frequencies of ileal γδ IELs from 12-17-wk-old Btnl2-KO and WT littermates. Data are pooled from 3 independent experiments with 3-6 mice/group. Error bars represent mean±SEM. Significance is measured using unpaired t-tests assuming similar SD, *p<0.05, ** p<0.005, significantly different from WT mice. FIG. 2E shows frequencies of γδ T cells in lamina propria (LP), mesenteric lymph nodes (MLN), spleen, and Peyer's Patches of 7-17-wk-old Btnl2-KO and WT littermates (n=3-6 mice/group).

FIGS. 3A, 3B, 3C, 3D, and 3E show that Btnl2 suppresses proliferation of jejunal/ileal γδ IELs. IELs were isolated from duodenum and jejunum/ileum of cohoused 12-wk-old Btnl2-KO and WT mice (n=4-5, each), labeled with CFSE and stimulated with α-CD3 and different Fc fusions in the presence of rh IL-2, rmIL7, rm IL15 for 84 hours. Supernatants from the cell cultures were collected, and cells were processed for flow cytometry. FIG. 3A shows representative flow cytometry plots of duodenal and jejunal/ileal WT CD8αα+γδ IELs following 84 hours of culture in the presence of equimolar concentrations of Btnl2-Fc and control mFc fusion proteins. FIGS. 3B and 3C show suppression of proliferation calculated as the percent difference between the proliferation in the presence of a specific Fc fusion and no Fc fusion, relative to the proliferation in the absence of Fc fusion. FIG. 3B shows suppression of proliferation of duodenal and jejunal/ileal WT CD8αα+γδ IELs and CD8αβ+αβ IELs. FIG. 3C shows suppression of proliferation of duodenal and jejunal/ileal Btnl2-KO CD8αα+γδ IELs and CD8αβ+αβ IELs. Error bars represent mean+/− SEM. Significance is measured using one-way ANOVA, *p<0.05, ** p<0.005, *** p<0.001, **** p<0.0001, significantly different from mFc fusion protein control. FIGS. 3D and 3E show that cohoused 11-wk-old Btnl2-KO and WT mice (n=4-5, each) were given BrdU at 0.8 mg/mL ad libitum in drinking water for 3 days. BrdU incorporation was measured by intranuclear staining of γδ IELs from different segments of small intestine over time. FIG. 3D shows representative flow cytometry plots of BrdU incorporation in ileal CD8αα+γδ IELs. FIG. 3E shows BrdU incorporation in CD8αα+γδ IELs and CD8αβ+αβ IELs across different segments. Error bars represent mean+/− SEM. Significance is measured using unpaired t-tests assuming similar SD, *p<0.05, ** p<0.005, *** p<0.0005, **** p<0.0001, significantly different from WT.

FIGS. 4A, 4B, 4C, and 4D show that ileal Btnl2-KO γδ IELs display an altered antibacterial response module compared to ileal WT γδ IELs. γδ IELs from duodenum, jejunum, and ileum of cohoused 11-wk-old Btnl2-KO and WT littermates (n=3-4/genotype, each a pool of 2 mice) were sort-purified as CD45+TCRB-TCRγδ+ cells. RNA sequencing was performed, and gene set enrichment analysis using NextBio. Gene ontology (GO) was employed to identify GO biological processes differentially enriched in Btnl2-KO and WT γδ IELs. FIG. 4A shows a volcano plot displaying genes differentially regulated between ileal Btnl2-KO and WT γδ IELs. The horizontal dashed line indicates FDR-0.05, and the vertical dashed line indicates |Fold Change|=1.5. FIG. 4B shows hierarchical clustering of top 50 differentially expressed genes between ileal Btnl2-KO and WT γδ IELs; duodenal and jejunal Btnl2-KO and WT γδ IELs were also included as a comparison. FIG. 4C shows gene ontology enrichment analysis of most significantly impaired biological processes in ileal Btnl2-KO γδ IELs compared to ileal WT γδ IELs. FIG. 4D shows hierarchical clustering of top dysregulated antibacterial response genes in ileal Btnl2-KO and WT γδ IELs within the combined top 3 GO processes; duodenal and jejunal Btnl2-KO and WT γδ IELs were also included as a comparison.

FIGS. 5A, 5B, 5C, and 5D show that ileal Btnl2-KO γδ IELs exhibit a more diverse TRGV repertoire compared to ileal WT γδ IELs. γδ IELs from duodenum, jejunum, and ileum of cohoused 11-wk-old Btnl2-KO and WT littermates (n=3-4/genotype, pool of 2 mice, each) were sort-purified as CD45+TCRB-TCRγδ+ cells. Two thirds of each sample were processed for deep bulk RNA sequencing, and one third of each sample was pooled per genotype per segment and used for single-cell sorting and single-cell TCR sequencing. Γδ IELs from duodenum, jejunum, and ileum of cohoused 11-wk-old Btnl2-KO and WT littermates (n=8 mice, each) were single-cell sorted, and single cell TCR sequencing analysis of TCR Vγ and TCR Vδ chain usage and CDR3 amino acid sequences was performed. FIG. 5A shows TRGV*J and TRDV*J gene usage in Btnl2-KO and WT γδ IELs. ND—not detected. FIG. 5B (top panel) shows diversity estimates for TRG only CDR3 amino acid sequences in Btnl2-KO and WT γδ IELs. Shaded areas indicate the 95% confidence interval by 50 bootstrap replicates. FIG. 5B (bottom panel) shows TRG diversity estimates at interpolation point 3500, where p value is derived from t-test based on 50 bootstrap replicates. FIG. 5C shows diversity estimates for TRD only and paired TRG and TRD CDR3 amino acid sequences, respectively, in ileal Btnl2-KO and WT γδ IELs. Shaded areas indicate the 95% confidence interval by 50 bootstrap replicates. FIG. 5D shows diversity 50 (D50) index represented as the number of top unique clones that comprise 50% of the TRGV and TRDV repertoires, respectively, normalized to the total number of unique clones of duodenal, jejunal and ileal Btnl2-KO and WT γδ IELs.

FIGS. 6A, 6B, 6C, 6D, and 6E show that ileal γδ IEL transcriptome of shared Btnl2-KO and WT CDR3γ clones is shaped by pairing with CDR3δ. Ileal γδ IELs from cohoused 11-wk-old Btnl2-KO and WT littermates (pool of 8 mice, each) were single-cell sorted, and single-cell TCR sequencing and single-cell RNA sequencing were performed. FIG. 6A shows top 20 TRG clones (CDR3γ amino acid sequences listed in order on the right and provided as follows: CASWAGYSSGFHKVF (SEQ ID NO: 29); CSYGLYSSGFHKVF (SEQ ID NO: 30); CASWAYSSGFHKVF (SEQ ID NO: 31); CASWGYSSGFHKVF (SEQ ID NO: 32); CAVWMRYSSGFHKVF (SEQ ID NO: 33); CAVWIGTSWVKIF (SEQ ID NO: 34); CASWAGRSSGFHKVF (SEQ ID NO: 35); CASWAVYSSGFHKVF (SEQ ID NO: 36); CASWAPYSSGFHKVF (SEQ ID NO: 37); CASWVYSSGFHKVF (SEQ ID NO: 38); CASWAGSSGFHKVF (SEQ ID NO: 39); CASWADSSGFHKVF (SEQ ID NO: 40); CASWAGGSSGFHKVF (SEQ ID NO: 41); CASWAGKYSSGFHKVF (SEQ ID NO: 42); CASWAEYSSGFHKVF (SEQ ID NO: 43); CAVWVYSSGFHKVF (SEQ ID NO: 44); CAVWGYSSGFHKVF (SEQ ID NO: 45); CAVWNSSGFHKVF (SEQ ID NO: 46); CASWAGDSSGFHKVF (SEQ ID NO: 47); CASWALYSSGFHKVF (SEQ ID NO: 48)) from ileal Btnl2-KO γδ IELs, which are differentially enriched in ileal WT γδ IELs. FIG. 6B shows top largest CDR3γ clones identified during scTCRseq can be found among individual ileal Btnl2-KO (n=3) and ileal WT (n=4) samples, in which TCR sequences have been reconstructed from bulk RNAseq and provided as follows: CASWAGYSSGFHKVF (SEQ ID NO: 29); CASWGYSSGFHKVF (SEQ ID NO: 32); CASWAYSSGFHKVF (SEQ ID NO: 31); CASWEYSSGFHKVF (SEQ ID NO: 49); CASSSGFHKVF (SEQ ID NO: 50); CASWAGRSSGFHKVF (SEQ ID NO: 35); CASWAVYSSGFHKVF (SEQ ID NO: 36); CASWAGHSSGFHKVF (SEQ ID NO: 51); CAVWVYSSGFHKVF (SEQ ID NO: 44); CASWAGDSSGFHKVF (SEQ ID NO: 47); CASWAGYSSGFHKVF (SEQ ID NO: 29); CSYGLYSSGFHKVF (SEQ ID NO: 30); CASWAYSSGFHKVF (SEQ ID NO: 31); CASWGYSSGFHKVF (SEQ ID NO: 32); CAVWMRYSGFHKVFF (SEQ ID NO: 52); CASWAGRSSGFHKVF (SEQ ID NO: 35); CASWAVYSSGFHKVF (SEQ ID NO: 36); CASWAPYSSGFHKVF (SEQ ID NO: 37); CASWVYSSGFHKVF (SEQ ID NO: 38). Shared clones are highlighted in bold. Each slice represents a different sample, and white slices mark the absence of the CDR3γ clone from the individual sample. FIG. 6C shows multiple Vγ7-J1 recombination events converge to the same top 1 CDR3γ amino acid sequence (CASWAGYSSGFHKVF (SEQ ID NO: 29)) in both ileal Btnl2-KO and ileal WT γδ IELs. FIG. 6D shows the distribution of top 1 CDR3Y chain (TRGV7*02/TRG11*01, CASWAGYSSGFHKVF (SEQ ID NO: 29); 10% of total clones) shared by ileal Btnl2-KO and ileal WT γδ IELs among different UMAP clusters. FIG. 6E shows pairing of top 1 CDR3γ (CASWAGYSSGFHKVF (SEQ ID NO: 29)) with different CDR38 sequences provides as follows: CALMERGISEGYSTDKLVF (SEQ ID NO: 53); CALWEPIWHIGGIRATDKLVF (SEQ ID NO: 54); CALMERIGLTTDKLVF (SEQ ID NO: 55); AMGTLSEGYELGDKLVF (SEQ ID NO: 56); CASGFLWHLYRRDTSPTDKLVF (SEQ ID NO: 57); CALMELSEGPATDKLVF (SEQ ID NO: 58); CASGYTVWQIGGIRATDKLVF (SEQ ID NO: 59); CALSEPSAVDRRDTKTDKLVF (SEQ ID NO: 60), listed above the UMAP plot, shapes the transcriptome of the γδ IELs. Numbers of clones per pair are denoted below the CDR3 γ sequences.

FIGS. 7A, 7B, 7C, 7D, 7E, 7F, and 7G show that Btnl2-KO mice exhibit more severe intestinal inflammation in chronic DSS-induced colitis. Cohoused 15-wk-old Btnl2-KO (n=11) and WT (n=8) littermates were subjected to 3% DSS-induced colitis for 7 days followed by water for 8 days. Control mice (n=2-4) received water. FIG. 7A shows body weight loss in cohoused Btnl2-KO and WT littermates calculated as the percent difference between the initial and actual body weight on the above days. Error bars represent mean+/− SEM. Significance is measured using unpaired t-tests assuming similar SD, *p<0.05, ** p<0.005, *** p<0.0005, significantly different from DSS-treated WT mice. FIG. 7B shows colon length of water- and DSS-treated Btnl2-KO and WT mice on day 15. FIG. 7C shows H&E histological sections and a pathological score of colon from water- and DSS-treated Btnl2-KO and WT mice. FIG. 7D shows myeloperoxidase (MPO) activity in colon homogenates of water- and DSS-treated Btnl2-KO and WT mice. FIG. 7E shows levels of pro-inflammatory cytokines in colon homogenates of water and DSS-treated Btnl2-KO and WT mice. FIG. 7F shows granzyme A mRNA levels in colon homogenates of water- and DSS-treated Btnl2-KO and WT mice, normalized to β2m. Error bars represent mean+/− SEM. Significance is measured using unpaired t-tests assuming similar SD, *p<0.05. FIG. 7G shows Btnl1/2/6 mRNA levels in colon of water- and DSS-treated WT mice, normalized to β2m. Error bars represent mean+/− SEM. Significance is measured using one-way ANOVA, *p<0.05, ** p<0.005, *** p<0.0005.

FIG. 8 shows that expression of Btnl2 neighboring genes is not significantly affected by Btnl2-LacZ deletion cassette. Top panel-schematic representation of mouse Chr. 17qB1 locus based on USCS genome browser, showing the orientation and position of the genes near Btnl2 locus. Bottom panel: RNAseq reads (TPM) around the Btnl2 locus in intestinal epithelial cells isolated from different segments of the small intestine of cohoused 7-wk-old Btnl2-KO and WT mice (n=2-4, each). Error bars represent mean±SEM.

FIGS. 9A, 9B, and 9C show that Btnl2-KO mice do not develop spontaneous intestinal inflammation at steady-state. Duodenum, jejunum, and ileum were collected from 7- and 17-wk-old cohoused Btnl2-KO and WT mice (n=4-7, each) and processed for RNAseq and histology. FIG. 9A shows representative H&E staining and histological analysis of duodenum, jejunum, and ileum of 17-wk-old Btnl2-KO and WT littermates. FIG. 9B shows pro-inflammatory cytokines (TPM) in the ileum of 17-wk-old Btnl2-KO and WT littermates. Error bars represent mean±SEM. FIG. 9C shows intestinal epithelial cells (IECs) from jejunum/ileum of cohoused 7-wk-old Btnl2-KO and WT mice were processed for RNAseq. Plots represent fold change of Btnl2-KO reads relative to WT reads (grey bars) and their matching p-value (black line) in genes associated with differentiation and maturation of intestinal epithelial cells.

FIGS. 10A, 10B, and 10C show that Btnl2-KO mice exhibit comparable immune phenotypes in small intestine at a steady state. Ileum, Peyer's Patches, and mesenteric lymph nodes were collected from cohoused 7-17-wk-old Btnl2-KO and WT mice (n=3-13, each, pool of 1-3 separate experiments) and processed for flow cytometry. FIGS. 10A, 10B, and 10C show frequencies of major lymphocyte subsets within the ileal lamina propria (FIG. 10A), mesenteric lymph nodes (FIG. 10B), and Peyer's Patches (FIG. 10C). Error bars represent mean+/− SEM. Significance is measured using unpaired t-tests assuming similar SD, *p<0.05, ** p<0.005, *** p<0.0005, significantly different from WT. Tregs, regulatory T cells. DCs, dendritic cells. ILC3, innate lymphoid cells type 3. Dendritic cells are gated on lineage (TCRβ, B220, and Ly6c) negative cells. ILC3 cells are also gated on lineage (CD11b, CD11c, Gr1, B220, TCRβ, and NK1.1) negative cells.

FIGS. 11A, 11B, 11C, and 11D show that Btnl2 suppresses activation and proliferation of CD4⁺T cells comparably to Pdl1 and Pdl2 in vitro. Total CD4+ T cells were enriched from the spleens and lymph nodes of 10-12-wk-old WT mice (n=5, each), labeled with CFSE, and stimulated with α-CD3 or α-CD3/α-CD28 in the presence of different Fc fusions for 72 hours. At 72 hours, culture supernatants were collected, and cells were processed for flow cytometry. FIG. 11A shows the schematic design of the in vitro CD4+T cell proliferation assay. FIG. 11B shows a set of graphs displaying activation and suppression of proliferation of CD4+T cells following 72 hours of culture. Activation is represented as the frequency of CD44+CD4+T cells. Suppression of proliferation is calculated as the percent difference between the proliferation in the presence of a specific Fc fusion and no Fc fusion, relative to the proliferation in the absence of Fc fusion. FIG. 11C shows levels of pro-inflammatory cytokines in supernatants derived from CD4+T cell cultures with α-CD3 or α-CD3/α-CD28 and equimolar concentrations of Fc fusion proteins for 72 hours. FIG. 11D shows histograms displaying putative Btnl2 receptor staining and median fluorescence intensity on activated CD4+T cells at 72 hours following α-CD3 or α-CD3/α-CD28 stimulation. Right panel-Median fluorescence intensity of Fc-fusion-stained CD4+T cells. Error bars represent mean+/− SEM. One-way ANOVA with Tukey's test for multiple comparisons, *p<0.05, ** p<0.005, *** p<0.001, **** p<0.0001.

FIGS. 12A, 12B, and 12C show that jejunal/ileal Btnl2-KO and WT γδ IELs exhibit comparable expression levels of coinhibitory receptors and markers of maturation and activation. IELs were isolated from jejunum/ileum of cohoused 12-13-wk-old Btnl2-KO and WT (pool of 6 mice, each) and processed for flow cytometry by staining with LEGENDScreen mouse cell antibody panel (Biolegend, CA). Cells are gated on CD45+TCRB-TCRγδ+CD8αα+. FIG. 12A shows the schematic design of the in vitro IEL proliferation assay. FIG. 12B shows representative flow cytometry plots of duodenal and jejunal/ileal Btnl2-KO CD8αα+γδ IELs following 84 hours of culture in the presence of equimolar concentrations of Btnl2-Fc and control mFc fusion proteins. FIG. 12C shows histograms of maturation, activation, and co-inhibitory receptors for Btnl2-KO and WT CD8αα+γδ IELs. MFI, mean fluorescence intensity.

FIGS. 13A, 13B, 13C, and 13D show that Btnl2-KO γδ IELs have diverse TRGV and TRDV repertoire across small intestinal segments. τδ IELs from duodenum, jejunum, and ileum of cohoused 11-wk-old Btnl2-KO and WT littermates (n=3-4/genotype, pool of 2 mice, each) were sort-purified as CD45+TCRB-TCRγδ+ cells. Two thirds of each sample were processed for deep bulk RNA sequencing, and one third of each sample was pooled per genotype per segment and used for single-cell sorting and single-cell RNA sequencing. FIGS. 13A and 13B show Treemap rendering of all unique CDR3γ (FIG. 13A) or CDR3δ (FIG. 13B) amino acid sequences/combined sample, in which the size of the box corresponds to the size of the CDR3γ or CDR3δ clone, with the TRGV/TRGJ or TRDV/TRDJ genes used indicated, including TRGV1_TRGJ4, TRGV2_TRGJ2, TRGV3_TRGJ3, TRGV4_TRGJ1, TRGV5_TRGJ1, TRGV6 TRGJ1, and TRGV7_TRGJ1 (FIG. 13A); 1, and TRAV13/DV7_TRDJ1, TRAV13/DV3_TRDJ1, TRAV15/DV5_TRDJ1, TRAV5/DV3_TRDJ2, TRAV16/DV11_TRDJ1, TRAV21/DV12_TRDJ1, TRAV21/DV12_TRDJ2, TRDV1_TRDJ1, TRDV1_TRDJ2, TRDV2_TRDJ1, TRDV2_TRDJ2, TRDV4_TRDJ1, TRDV4_TRDJ2, TRDV5_TRDJ1, and TRDV5_TRDJ2. FIG. 13C shows Venn diagrams displaying the number of overlapping CDR3Y and CDR3δ clones per segment. FIG. 13D shows Venn diagrams displaying the number of overlapping CDR3γ and CDR3δ clones per genotype.

FIGS. 14A, 14B, and 14C show that CDR3γ/CDR3δ clone repertoire is unique to each small intestine segment and genotype. τδ IELs from duodenum, jejunum, and ileum of cohoused 11-wk-old Btnl2-KO and WT littermates (n=3-4/genotype, pool of 2 mice, each) were sort-purified as CD45+TCR8-TCRγδ+ cells. Two thirds of each sample were processed for deep bulk RNA sequencing, and one third of each sample was pooled per genotype per segment and used for single-cell sorting and single-cell RNA sequencing. FIG. 14A shows Treemap rendering of all unique pairs of CDR3γ and CDR3δ aminoacid sequences/combined sample, in which the size of the box corresponds to the size of the paired CDR3γ/CDR3δ clone with the TRGV/TRGJ genes used indicated, including TRGV1_TRGJ4, TRGV2_TRGJ2, TRGV3_TRGJ3, TRGV4_TRGJ1, TRGV5_TRGJ1, TRGV6_TRGJ1, and TRGV7_TRGJ1.

FIG. 14B shows Venn diagrams displaying the number of overlapping paired CDR3γ/CDR3δ clones per segment. FIG. 14C shows Venn diagrams displaying the number of overlapping paired CDR3γ/CDR3δ clones per genotype.

FIGS. 15A, 15B, 15C, and 15D show that γδ IELs can be subdivided into different clusters based on developmental origin, differentiation stage, and effector profile. Γδ IELs (CD45+TCRβ−TCRγδ+) from duodenum, jejunum, and ileum of cohoused 11-wk-old Btnl2-KO and WT littermates (pool of 8 mice, each) were single-cell sorted, and single cell RNA sequencing and single cell TCR sequencing were performed. FIG. 15A shows a combined UMAP plot with clusters marking nine distinct clusters based on gene expression differences for >4400 cells/segment/genotype. Right panel-Proportion of sorted Btnl2-KO and WT γδ IEL single cells that segregate with each cluster. FIG. 15B shows individual UMAP clustering of single cell RNA sequenced duodenal, jejunal, and ileal Btnl2-KO and WT γδ IELs. FIG. 15C shows the top 20 genes and their mean Fold Change (FC) enrichment in clusters 0-7 relative to their expression in the whole single cell population. FIG. 15D shows the frequency of TRGV gene usage in different clusters in ileal Btnl2-KO and WT γδ IELs.

FIGS. 16A, 16B, 16C, and 16D show that Btnl2-KO mice exhibit comparable ileal immune responses to WT mice in chronic DSS-induced colitis. Cohoused 15-wk-old Btnl2-KO (n=11) and WT (n=8) littermates were subjected to 3% DSS-induced colitis for 7 days followed by water for 8 days. Control mice (n=2-4) received water. FIG. 16A shows myeloperoxidase (MPO) activity in ileal homogenates of water- and DSS-treated Btnl2-KO and WT mice. FIG. 16B shows transcript levels of pro-inflammatory cytokines in ileal homogenates of water and DSS-treated Btnl2-KO and WT mice. Error bars represent mean+/− SEM. Significance is measured using one-way ANOVA, *p<0.05, ** p<0.005. FIG. 16C shows granzyme A mRNA levels in ileal homogenates of water- and DSS-treated Btnl2-KO and WT mice, normalized to β2m. FIG. 16D shows Btnl1/2/6 mRNA levels in ileal homogenates of water- and DSS-treated Btnl2-KO and WT mice, normalized to β2m.

FIGS. 17A, 17B, 17C, and 17D show that Btnl2-mediated inhibition of CD4+T cell activation is independent of α-CD3/α-CD28 displacement. Total CD4⁺T cells were enriched from spleens and lymph nodes of 10-12-wk-old WT mice (n=5, each), labeled with CFSE, and stimulated with α-CD3 or α-CD3/α-CD28 in the presence of different Fc fusions for 72 hours. At 72 hours, culture supernatants were collected, and cells were processed for flow cytometry. FIG. 17A depicts a schematic design of in vitro CD4⁺T cell proliferation assay with concurrent or sequential coating of α-CD3 or α-CD3/α-CD28 with Fc fusion proteins. FIGS. 17B, 17C, and 17D show graphs that display activation and suppression of proliferation of CD4⁺T cells following 72 hours of culture. Activation is represented as the frequency of CD44 CD4⁺T cells. Suppression of proliferation is calculated as the percent difference between the proliferation in the presence of a specific Fc fusion and no Fc fusion, relative to the maximum proliferation. FIG. 17B depicts concurrent coating with equal moles of Fc fusion proteins. FIG. 17C depicts sequential coating with equal moles of Fc fusion proteins. FIG. 17D depicts concurrent coating with the highest mass of Fc fusion proteins. Error bars represent mean+/− SEM. One-way ANOVA with Tukey's test for multiple comparisons, *p<0.05, ** p<0.005, *** p<0.001, **** p<0.0001; control group is Fc (IgG2a).

FIGS. 18A, 18B, 18C, and 18D show that BTNL2 induces de novo CD103⁺ FoxP3⁺ CD4⁺ T cell differentiation comparably to PDL1 in vitro. Naive CD4⁺T cells were enriched from spleens and lymph nodes of 10-12-wk-old WT mice (n=3-4, each) and stimulated with α-CD3 or α-CD3/α-CD28 in the presence of different Fc fusions and lineage-skewing cytokines for 5 days. After 5 days, culture supernatants were collected, and cells were processed for flow cytometry. FIG. 18A shows a schematic design of the in vitro CD4⁺ T helper cell differentiation assay. FIG. 18B shows graphs displaying frequencies of CD103⁺ and CD103 FoxP3⁺ regulatory T cells following 5 days of differentiation in the presence of IL-2, TGFβ1, and different Fc fusion proteins. FIG. 18C shows histograms displaying Akt/mTOR pathway kinase staining and median fluorescence intensity on stimulated CD4⁺T cells at 15 hours in the presence of IL-2 and TGFβ1 and different Fc fusion proteins. FIG. 18D shows histograms displaying STAT1/5 phosphorylation and median fluorescence intensity on differentiated regulatory CD4⁺T cells at 20 min in the presence of IL-2 and varying concentrations of TGFβ1. Error bars represent mean+/− SEM. One-way ANOVA with Tukey's test for multiple comparisons, *p<0.05, ** p<0.005, *** p<0.001, **** p<0.0001; control group is Fc (IgG2a).

FIGS. 19A, 19B, 19C, 19D, 19E, and 19F show that Btnl2 skews Th17 cell differentiation towards FoxP3⁺ regulatory T cell differentiation similarly to PDL1 in vitro. Naive CD4⁺T cells were enriched from spleens and lymph nodes of 10-12-wk-old WT mice (n=3-4, each) and stimulated with α-CD3 or α-CD3/α-CD28 in the presence of different Fc fusions and lineage-skewing cytokines for 5 days. After 5 days, culture supernatants were collected, and cells were processed for flow cytometry. FIGS. 19A and 19E show graphs displaying frequencies of Th17 cells and FoxP3⁺ regulatory T cell subsets following 5 days of differentiation in the presence of (FIG. 19A) IL-2, TGFβ1, IL-6, and IL-23 or (FIG. 19E) IL-2, TGFβ1, IL-6, IL-1β and IL-23, and different Fc fusion proteins. FIGS. 19B and 19F show histograms displaying Akt/mTOR pathway kinase staining and median fluorescence intensity on stimulated CD4⁺T cells at 15 hours in the presence of (FIG. 19B) IL-2, TGFβ1, IL-6, and IL-23 or (FIG. 19F) IL-2, TGFβ1, IL-6, IL-1β and IL-23 and different Fc fusion proteins. FIG. 19C shows histograms displaying STAT1/5 phosphorylation and median fluorescence intensity on differentiated CD4⁺T cells at 20 min in the presence of indicated cytokines. FIG. 19D shows levels of proinflammatory cytokines/chemokines following 5 days of Th17 cell differentiation in the presence of IL-2, TGFβ1, IL-6, and IL-23 and different Fc fusions proteins. Error bars represent mean+/− SEM. One-way ANOVA with Tukey's test for multiple comparisons, *p<0.05, ** p<0.005, *** p<0.001, **** p<0.0001; control group is Fc (IgG2a).

FIGS. 20A and 20B illustrate an exemplary embodiment, showing effects of treatments with a Btnl2-Fc fusion protein in WT and Btnl2 KO mice with DSS-induced colitis. Btnl2 KO mice and cohoused WT littermates were subjected to chronic DSS-induced colitis by administrating 3% DSS for 6 days, followed by water for 6 days. Control mice (n=3) received regular water. mBTNL2-Fc and mFc were intraperitoneally injected at 25 mg/kg on days-1, 1, 4, 7, and 11. FIGS. 20A and 20B display body weight loss in WT and Btnl2 KO mice, respectively. Data are representative of one experiment. n=8-10 mice per treatment group. Error bars represent mean+/− SEM; * p<0.05, ** p<0.005, *** p<0.001, **** p<0.0001, as determined by two-way ANOVA with Tukey's multiple comparison test; control group is Fc.

### DETAILED DESCRIPTION

This disclosure provides methods and reagents for treating inflammatory disorders, based, in part, on an unexpected discovery that butyrophilin-like 2 (Btnl2) regulates intraepithelial lymphocytes (IELs), such as intestinal γδ IELs development and maintenance. Tissue-resident γδ IELs orchestrate both innate and adaptive immune responses to maintain intestinal epithelial barrier integrity. Epithelia-specific butyrophilin-like (Btnl) molecules induce perinatal development of distinct Vγ TCR IELs. However, the mechanisms that control γδ IEL maintenance within discrete intestinal segments are unclear. This disclosure demonstrates that Btnl2 suppresses homeostatic proliferation of γδ IELs preferentially in small intestinal segments, such as ileum. High throughput transcriptomic characterization of Btnl2-KO γδ IELs isolated from different segments of the small intestine revealed that Btnl2 regulated the antimicrobial response module of ileal γδ IELs. Btnl2 deficiency re-shaped the TCR specificities and TCRγ/8 repertoire diversity of ileal γδ IELs likely in tune with their local antigenic environment. During chronic DSS-induced colitis, Btnl2-KO mice exhibited increased inflammation and delayed mucosal repair in the colon. Collectively, these data indicated that Btnl2 fine-tunes γδ IEL frequencies and function as well as TCR specificities in response to site-specific homeostatic and inflammatory cues. Hence, Btnl-mediated targeting of IEL (e.g., γδ IEL) development and maintenance will help dissect their immunological functions in intestinal diseases with gut segment-specific manifestations.

a. Methods of Treating Inflammatory Disorders

In one aspect, this disclosure provides a method of treating an inflammatory disorder, including selecting a subject with an inflammatory disorder and an elevated level of intraepithelial lymphocytes (IELs) as compared to a predetermined reference value; and administering to the subject a therapeutically effective amount of a Btnl2 agent that is capable of decreasing proliferation of IELs in the subject by increasing a level or activity of Btnl2 in the subject.

In some embodiments, the subject has at least one loss-of-function single nucleic polymorphism (SNP) at rs28362675 in the Btnl2 gene. In some embodiments, the subject has a loss-of-function SNP at rs28362675 in the Btnl2 gene. In some embodiments, the subject has one loss-of-function SNP at rs28362675 in the Btnl2 gene. rs28362675 contains a SNP at position chr6:32394744 (GRCh38.p13). In some embodiments, the subject has at least one loss-of-function SNP at rs28362675 that results in a Glu454Ter (stop codon) mutation. In some embodiments, the subject has a loss-of-function SNP at rs28362675 that results in a Glu454Ter (stop codon) mutation. In some embodiments, the subject has one loss-of-function SNP at rs28362675 that results in a Glu454Ter (stop codon) mutation.

In some embodiments, the method comprises: (a) obtaining a sample containing IELs; (b) performing an assay on the sample and determining the number of the IELs in the sample; (c) identifying the subject having an increased number of IELs as compared to a predetermined reference value; and (d) administering to the subject a therapeutically effective amount of a Btnl2 agent capable of modulating (e.g., decreasing) the number of IELs in the subject by increasing a level or activity of Btnl2 in the subject.

In some embodiments, the lymphocytes comprise CD4+ T cells, CD8+T cells, regulatory T cells (Treg) or tissue-specific T cells. In some embodiments, the IELs comprise γδ T cells. In some embodiments, the IELs comprise intestinal γδ IELs. In some embodiments, the intestinal γδ IELs comprise jejunal γδ IELs, ileal γδ IELs, colonic γδ IELs, or combinations thereof. In some embodiments, the intestinal γδ IELs comprise the ileal γδ IELs. In some embodiments, the intestinal γδ IELs comprise ileal CD8αα+γδ IELs.

In some embodiments, the inflammatory disorder is an intestinal inflammatory disorder. In some embodiments, the inflammatory disorder is an immune-mediated disease, e.g., an immune-mediated inflammatory disease. In some embodiments, the inflammatory disorder is an autoimmune disease. In some embodiments, the inflammatory disorder is a gut-associated immune-mediated disease or a gut-associated autoimmune disease. In some embodiments, the immune-mediated disease or a gut-associated autoimmune disease comprises graft versus host disease (GVHD) (e.g., GVHD with or without intestinal manifestations), Celiac disease, ulcerative colitis, Crohn's disease, rheumatoid arthritis, sarcoidosis, myositis, inflammatory bowel disease, gastrointestinal inflammation, or type I diabetes. Non-limiting examples of intestinal manifestations include nausea, loss of appetite, a feeling of fullness, indigestion, gas, bloating, diarrhea, pain, weight loss and/or other possible indications of intestinal distress.

In some embodiments, the above-described method includes determining the level or activity of Btnl2 or the number of intestinal γδ IELs in the subject. The determined level or activity of Btnl2 or the determined number of intestinal γδ IELs may be compared to the level or activity of Btnl2 or the number of intestinal γδ IELs prior to the administration of the Btnl2 agent or a composition thereof to the subject or a predetermined reference value.

The level or activity of Btnl2 may be measured by determining or estimating a protein level or mRNA level. Methods for determining or estimating a protein level or mRNA level are well known in the art. For example, the protein level (e.g., protein expression level) of Btnl2 can be determined by SDS-PAGE, Western blot, or an immunoassay (e.g., immunoblotting assay, immunoprecipitation assay). The mRNA level may be determined by RT-PCR.

As used herein, the term “predetermined reference value” refers to a reference value that may be any of the following: (a) obtained from the subject prior to the administration of the agent for increasing a level or activity of Btnl2 or a composition thereof; (b) obtained from a control subject or a group of individuals (in which case an average is used as the reference value) who do not have an inflammatory disorder (e.g., gut-related inflammatory disorder, such as IBD) or have not been diagnosed with an inflammatory disorder; (c) obtained based on average levels of the level or activity of Btnl2 or the number of IELs (e.g., γδ IELs, such as intestinal γδ IELs) in a control population, for example, a population not suffering from an inflammatory disorder; or (d) obtained based on a median or median level of a set of individuals in which patients with an inflammatory disorder are included.

In some embodiments, the predetermined reference value may be obtained from the subject prior to the administration of the agent for increasing a level or activity of Btnl2 or a composition thereof. In some embodiments, the predetermined reference value may be obtained from a control subject or a group of individuals who do not have an inflammatory disorder (e.g., gut-related inflammatory disorder, such as IBD) or have not been diagnosed with an inflammatory disorder. In some embodiments, the predetermined value is obtained based on average levels of the level or activity of Btnl2 or the number of IELs (e.g., γδ IELs, such as intestinal γδ IELs) in a control population, for example, a population not suffering from an inflammatory disorder. In some embodiments, the predetermined value is obtained based on a median or median level of a set of individuals in which patients with an inflammatory disorder are included.

In some embodiments, a predetermined reference value can be determined by analyzing the number of IELs using flow cytometry (e.g., fluorescence activated cell sorting (FACS), such as FACSCalibur (Becton Dickinson) or LSRFortessa X-20 instrument (BD Biosciences)), as described in, e.g., WO2013077186A1, which is incorporated by reference herein in its entirety. In some embodiments, the number of IELs (e.g., jejunal IELs, ileal IELs, colonic IELs) can be counted and expressed as the number of IELs per 100 intestinal epithelial cells (“IEL/100”), as described in, e.g., DePaolo et al. Nature. 2011 Mar. 10; 471 (7337): 220-24, which is incorporated by reference herein in its entirety. In some embodiments, a predetermined reference value for the number of IELs as expressed in an IEL/100 value obtained from a healthy control may be from about 10 to about 25, such as 13+/−7, as described in, e.g., Rostami et al. Gut 2017; 66:2080-86, which is incorporated by reference herein in its entirety.

As used herein, the terms “increase,” “elevate,” “elevated,” “enhance,” and “activate” all generally refer to an increase by a statically significant amount as compared to a reference level.

For the avoidance of any doubt, these terms mean an increase of at least 5% (e.g., 5%, 10%, 15%, 20%, 25%, 30%, 35%, 40%, 45%, 50%, 55%, 60%, 65%, 70%, 75%, 80%, 85%, 90%, 95%, 100%) as compared to a reference level, for example, an increase of at least about 10%, at least about 20%, at least about 30%, at least about 40%, at least about 50%, at least about 60%, at least about 70%, at least about 80%, at least about 90% or at least about 100%, as compared to a reference level. In some embodiments, these terms may refer to an increase of 10-20%, 10-30%, 10-40%, 10-50%, 10-60%, 10-70%, 10-80%, 10-90%, 10-100%, 10-110%, 10-120%, 10-130%, 10-140%, 10-150%, 10-160%, 10-170%, 10-180%, 10-190%, 10-200%, 10-210%, 10-220%, 10-230%, 10-240%, 10-250%, 10-260%, 10-270%, 10-280%, 10-290%, or 10-300%, as compared to a reference level. In some embodiments, these terms may refer to an increase of 10-300%, 20-300%, 30-300%, 40-300%, 50-300%, 60-300%, 70-300%, 80-300%, 90-300%, 100-300%, 110-300%, 120-300%, 130-300%, 140-300%, 150-300%, 160-300%, 170-300%, 180-300%, 190-300%, 200-300%, 210-300%, 220-300%, 230-300%, 240-300%, 250-300%, 260-300%, 270-300%, 280-300%, or 290-300% as compared to a reference level. In some embodiments, these terms may refer to an increase of at least 2-fold, at least 3-fold, at least 4-fold, at least 5-fold, at least 10-fold or greater, as compared to a reference level.

In some embodiments, the method may include identifying or selecting a subject having an elevated level of IELs as compared to a predetermined reference value. The terms “predetermined value” and “predetermined level” are used interchangeably herein. Similarly, the terms “reference value” and “reference level” are used interchangeably herein. An elevated level of IELs as compared to a predetermined reference value can be an increase of at least 5% (e.g., 5%, 10%, 15%, 20%, 25%, 30%, 35%, 40%, 45%, 50%, 55%, 60%, 65%, 70%, 75%, 80%, 85%, 90%, 95%, 100%) as compared to a predetermined reference value, for example, an increase of at least about 10%, at least about 20%, at least about 30%, at least about 40%, at least about 50%, at least about 60%, at least about 70%, at least about 80%, at least about 90%, at least about 100%, or any increase from 10% to 100%, as compared to a predetermined reference value; or at least a 2-fold, at least a 3-fold, at least a 4-fold, at least a 5-fold or at least a 10-fold increase, or any increase from 2-fold to 10-fold or greater, as compared to a predetermined reference value.

In some embodiments, an “elevated” level of IELs as compared to a predetermined reference value can be an increase of 10-20%, 10-30%, 10-40%, 10-50%, 10-60%, 10-70%, 10-80%, 10-90%, 10-100%, 10-110%, 10-120%, 10-130%, 10-140%, 10-150%, 10-160%, 10-170%, 10-180%, 10-190%, 10-200%, 10-210%, 10-220%, 10-230%, 10-240%, 10-250%, 10-260%, 10-270%, 10-280%, 10-290%, or 10-300% as compared to a predetermined reference value. In some embodiments, an elevated level of IELs as compared to a predetermined reference value can be an increase of 10-300%, 20-300%, 30-300%, 40-300%, 50-300%, 60-300%, 70-300%, 80-300%, 90-300%, 100-300%, 110-300%, 120-300%, 130-300%, 140-300%, 150-300%, 160-300%, 170-300%, 180-300%, 190-300%, 200-300%, 210-300%, 220-300%, 230-300%, 240-300%, 250-300%, 260-300%, 270-300%, 280-300%, or 290-300% as compared to a predetermined reference value. In some embodiments, an elevated level of IELs as compared to a predetermined reference value can be an increase of 10-20%, 20-30%, 30-40%, 40-50%, 50-60%, 60-70%, 70-80%, 80-90%, 90-100%, 100-110%, 110-120%, 120-130%, 130-140%, 140-150%, 150-160%, 160-170%, 170-180%, 180-190%, 190-200%, 200-210%, 210-220%, 220-230%, 230-240%, 240-250%, 250-260%, 260-270%, 270-280%, 280-290%, or 290-300% as compared to a predetermined reference value.

As used herein, the terms “decrease,” “reduce,” and “inhibit” all generally refer to a decrease by a statistically significant amount. However, for avoidance of doubt, the term “reduced,” “decrease,” “reduce,” or “inhibit” means a decrease by at least 5% (e.g., 5%, 10%, 15%, 20%, 25%, 30%, 35%, 40%, 45%, 50%, 55%, 60%, 65%, 70%, 75%, 80%, 85%, 90%, 95%, 100%) as compared to a reference level, for example, a decrease by at least about 10%, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease of 10-100% as compared to a reference level. In some embodiments, these terms refer to a decrease of 10-20%, 10-30%, 10-40%, 10-50%, 10-60%, 10-70%, 10-80%, 10-90%, 10-100%, 10-110%, 10-120%, 10-130%, 10-140%, 10-150%, 10-160%, 10-170%, 10-180%, 10-190%, 10-200%, 10-210%, 10-220%, 10-230%, 10-240%, 10-250%, 10-260%, 10-270%, 10-280%, 10-290%, or 10-300%, as compared to a reference level. In some embodiments, these terms refer to a decrease of 10-300%, 20-300%, 30-300%, 40-300%, 50-300%, 60-300%, 70-300%, 80-300%, 90-300%, 100-300%, 110-300%, 120-300%, 130-300%, 140-300%, 150-300%, 160-300%, 170-300%, 180-300%, 190-300%, 200-300%, 210-300%, 220-300%, 230-300%, 240-300%, 250-300%, 260-300%, 270-300%, 280-300%, or 290-300%, as compared to a reference level. In some embodiments, these terms refer to a decrease of 10-20%, 20-30%, 30-40%, 40-50%, 50-60%, 60-70%, 70-80%, 80-90%, 90-100%, 100-110%, 110-120%, 120-130%, 130-140%, 140-150%, 150-160%, 160-170%, 170-180%, 180-190%, 190-200%, 200-210%, 210-220%, 220-230%, 230-240%, 240-250%, 250-260%, 260-270%, 270-280%, 280-290%, or 290-300%, as compared to a reference level.

The terms “subject” and “patient” may be used interchangeably herein, irrespective of whether the subject has or is currently undergoing any form of treatment. These terms may refer to any vertebrate, including, but not limited to, a mammal (e.g., cow, pig, camel, llama, horse, goat, rabbit, sheep, hamsters, guinea pig, cat, dog, rat, and mouse), a non-human primate (for example, a monkey, such as a cynomolgus monkey, chimpanzee, etc.) and a human. The subject may be a human or a non-human. In more exemplary aspects, the mammal is a human.

As used herein, the expression “a subject in need thereof” or “a patient in need thereof” means a human or non-human mammal that exhibits one or more symptoms or indications of an inflammatory disorder (e.g., a gut-related inflammatory disorder), and/or who has been diagnosed with an inflammatory disorder. In some embodiments, the subject is a mammal. In some embodiments, the subject is human.

The terms “treatment,” “treating,” “palliating,” or “ameliorating” may be used interchangeably herein. These terms refer to an approach for obtaining beneficial or desired results, including but not limited to a therapeutic benefit and/or a prophylactic benefit. By therapeutic benefit is meant any therapeutically relevant improvement in or effect on one or more diseases (e.g., inflammatory diseases), conditions, or symptoms under treatment. For prophylactic benefit, the agent or the compositions thereof may be administered to a subject at risk of developing a particular disease, condition, or symptom, or to a subject reporting one or more of the physiological symptoms of a disease, even though the disease, condition, or symptom may not have yet been manifested.

The terms “sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest, such as antibodies. The sample may be any tissue sample from the subject. The sample may comprise protein from the subject.

In another aspect, this disclosure provides a method of treating an inflammatory bowel disease (IBD), comprising: (a) selecting a subject having an IBD and at least one loss-of-function SNP at rs28362675 in the Btnl2 gene; and (b) administering to the subject a therapeutically effective amount of a Btnl2 agent capable of increasing a level or activity of Btnl2 in the subject.

In some embodiments, the subject has at least one loss-of-function SNP at rs28362675 that results in a Glu454Ter (stop codon) mutation. In some embodiments, the subject has a loss-of-function SNP at rs28362675 that results in a Glu454Ter (stop codon) mutation. In some embodiments, the subject has one loss-of-function SNP at rs28362675 that results in a Glu454Ter (stop codon) mutation. In some embodiments, the Btnl2 agent comprises a Btnl2 protein or variant thereof or a fusion protein comprising the Btnl2 protein or variant thereof.

In some embodiments, the method comprises: (i) obtaining from the subject a sample containing a nucleic acid; (ii) analyzing the sample and detecting the presence of at least one (e.g., 1, 2, 3, 4, 5) loss-of-function SNP at rs28362675 in the Btnl2 gene; (iii) identifying the subject having the at least one (e.g., 1, 2, 3, 4, 5) loss-of-function SNP at rs28362675 in the Btnl2 gene as having an increased risk of having or developing an inflammatory bowel disease; and (iv) administering to the subject a therapeutically effective amount of a Btnl2 agent capable of increasing a level or activity of Btnl2 in the subject. In some embodiments, the step of analyzing the sample comprises performing a genotyping assay on the sample.

In another aspect, this disclosure additionally provides a method of identifying a subject having an increased risk of having or developing an IBD. In some embodiments, the method comprises: (i) obtaining a sample containing a nucleic acid from the subject; (ii) analyzing the sample and detecting the presence of at least one (e.g., 1, 2, 3, 4, 5) loss-of-function SNP at rs28362675 in the Btnl2 gene; and (iii) identifying the subject having the at least one (e.g., 1, 2, 3, 4, 5) loss-of-function SNP at rs28362675 in the Btnl2 gene as having an increased risk of having or developing an IBD.

In some embodiments, the step of analyzing the sample comprises performing a genotyping assay on the sample.

In another aspect, this disclosure also provides a method of reducing the number of IELs in a subject, comprising administering an effective amount of a Btnl2 agent to a subject in need thereof, the Btnl2 agent capable of increasing a level or activity of Btnl2 in the subject.

In some embodiments, the lymphocytes comprise CD4+ T cells, CD8+T cells, regulatory T cells (Treg) or tissue-specific T cells. In some embodiments, the IELs comprise γδ T cells. In some embodiments, the IELs comprise intestinal γδ IELs. In some embodiments, the intestinal γδ IELs comprise jejunal γδ IELs, ileal γδ IELs, colonic γδ IELs, or combinations thereof. In some embodiments, the intestinal γδ IELs comprise the ileal γδ IELs. In some embodiments, the intestinal γδ IELs comprise ileal CD8αα+γδ IELs.

b. Btnl2 Agents for Treating Inflammatory Disorders

In some embodiments, the Btnl2 agent comprises a Btnl2 protein or variant thereof or a fusion protein comprising the Btnl2 protein or variant thereof.

In some embodiments, the fusion protein comprises a Btnl2-Fc fusion protein. In some embodiments, the immunoglobulin Fc region is an immunoglobulin Fc fragment that can be derived from IgG, IgA, IgD, IgE, or IgM. In some embodiments, each domain of the immunoglobulin Fc fragment can be a hybrid of domains, and each domain can have a different origin derived from immunoglobulins selected from the group consisting of IgG, IgA, IgD, IgE, and IgM. In some embodiments, the immunoglobulin Fc fragment can be a dimer or multimer consisting of single-chain immunoglobulins comprising domains having the same origin. IgG may be divided into the IgG1, IgG2, IgG3, and IgG4 subclasses, and may include combinations or hybrids thereof.

In some embodiments, the Btnl2 protein comprises an amino acid sequence having at least 90% (e.g., 90%, 91%, 92%, 93%, 94%, 95%, 96%, 97%, 98%, 99%) identity to an amino acid sequence of SEQ ID NOs: 1-3 or comprises an amino acid sequence of SEQ ID NOs: 1-3.

TABLE 1
Example Sequences

| SEQ | | |
|---|---|---|
| ID NO | SEQUENCE | NOTES |
| 1 | MVDFPGYNLSGAVASFLFILLTMKQSEDFR VIGPAHPILAGVG | Human |
| | EDALLTCQLLPKRTTMHVEVRWYRSEPSTPVFVHRDGVEVTE | Btnl2 (full |
| | MQMEEYRGWVEWIENGIAKGNVALKIHNIQPSDNGQYWCHF | length) |
| | QDGNYCGETSLLLKVAGLGSAPSIHMEGPGESGVQLVCTARG | NM_001304561.1 |
| | WFPEPQVYWEDIRGEKLLAVSEHRIQDKDGLFYAEATLVVRN | |
| | ASAESVSCLVHNPVLTEEKGSVISLPEKLQTELASLKVNGPSQ | |
| | PILVRVGEDIQLTCYLSPKANAQSMEVRWDRSHRYPAVHVY | |
| | MDGDHVAGEQMAEYRGRTVLVSDAIDEGRLTLQILSARPSD | |
| | DGQYRCLFEKDDVYQEASLDLKVVSLGSSPLITVEGQEDGEM | |
| | QPMCSSDGWFPQPHVPWRDMEGKTIPSSSQALTQGSHGLFHV | |
| | QTLLRVTNISAVDVTCSISIPFLGEEKIATFSLSESRMTFLWKTL | |
| | LVWGLLLAVAVGLPRKRS | |
| 2 | MVDFPGYNLSGAVASFLFILLTMKQSGLGSAPSIHMEGPGESG | Human |
| | VQLVCTARGWFPEPQVYWEDIRGEKLLAVSEHRIQDKDGLFY | Btnl2 |
| | AEATLVVRNASAESVSCLVHNPVLTEEKGSVISLPEKLQTELA | (isoform 1) |
| | SLKVNGPSQPILVRVGEDIQLTCYLSPKANAQSMEVRWDRSH | |
| | RYPAVHVYMDGDHVAGEQMAEYRGRTVLVSDAIDEGRLTL | |
| | QILSARPSDDGQYRCLFEKDDVYQEASLDLKVVSLGSSPLITV | |
| | EGQEDGEMQPMCSSDGWFPQPHVPWRDMEGKTIPSSSQALT | |
| | QGSHGLFHVQTLLRVTNISAVDVTCSISIPFLGEEKIATFSLSES | |
| | RMTFLWKTLLVWGLLLAVAVGLPRKRS | |
| 3 | MVDFPGYNLSGAVASFLFILLTMKQSEKLQTELASLKVNGPS | Human |
| | QPILVRVGEDIQLTCYLSPKANAQSMEVRWDRSHRYPAVHV | Btnl2 |
| | YMDGDHVAGEQMAEYRGRTVLVSDAIDEGRLTLQILSARPS | (isoform 2) |
| | DDGQYRCLFEKDDVYQEASLDLKVVSLGSSPLITVEGQEDGE | |
| | MQPMCSSDGWFPQPHVPWRDMEGKTIPSSSQALTQGSHGLF | |
| | HVQTLLRVTNISAVDVTCSISIPFLGEEKIATFSLSESRMTFLW | |
| | KTLLVWGLLLAVAVGLPRKRS | |
| 4 | atggtggattttccaggctacaatctgtctggtgcagtcgcctccttcctattcatcctgctgacaa | Human |
| | tgaagcagtcagaagactttagagtcattggccctgctcatcctatcctggccggggttggggaaga | Btnl2 (full |
| | tgccctgttaacctgccagctactccccaagaggaccacaatgcacgtggaggtgaggtggtacc | length) |
| | gctcagagcccagcacacctgtgtttgtgcacagggatggagtggaggtgactgagatgcagat | NG_054759 |
| | ggaggagtacagaggctgggtagagtggatagagaatggcattgcaaagggaaatgtggcact | |
| | gaagatacacaacatccagccctccgacaatggacaatactggtgccatttccaggatgggaact | |
| | actgtggagaaacaagcttgctgctcaaagtagcaggtctggggtctgcccctagcatccacatg | |
| | gagggacctggggagagtggagtccagcttgtgtgcactgcaaggggctggttcccagagccc | |
| | caggtgtattgggaagacatccggggagagaagctgctggccgtgtctgagcatcgcatccaag | |
| | ataaagatggcctgttctatgcggaagccaccctggtggtcaggaacgcctctgcagagtctgtgt | |
| | cctgcttggtccacaaccccgtcctcactgaggagaaggggtcggtcatcagcctcccagagaa | |
| | actccagactgagctggcttctttaaaagtgaatggaccttcccagcccatcctcgtcagagtggg | |
| | agaagatatacagctaacctgttacctgtcccccaaggcgaatgcacagagcatggaggtgaggt | |
| | gggaccgatcccaccgttaccctgctgtgcatgtgtatatggatggggaccatgtggctggagag | |
| | cagatggcagagtacagagggaggactgtactggtgagtgacgccattgacgagggcagactg | |
| | accctgcagatactcagtgccagaccttcggacgacgggcagtaccgctgcctttttgaaaaagat | |
| | gatgtctaccaggaggccagtttggatctgaaggtggtaagtctgggttcttccccactgatcactg | |
| | tggaggggcaagaagatggagaaatgcagccgatgtgctcttcagatgggtggttcccacagcc | |
| | ccacgtgccatggagggacatggaaggaaagacgataccatcatcttcccaggccctgactcaa | |
| | ggcagccacgggctgttccacgtgcagacattgctaagggtcacaaacatctccgctgtggacgtca | |
| | cttgttccatcagcatcccctttttgggcgaggagaaaatcgcaactttttctctctcagagtccag | |
| | gatgacgtttttgtggaaaacactgcttgtttggggattgcttcttgctgtggctgtaggcctgccc | |
| | aggaagaggagctga | |
| 5 | ccgggtgtgtcacagcctcacttaggcacagcgtttccttgtctttgttccagggaggatggtggat | Human |
| | tttccaggctacaatctgtctggtgcagtcgcctccttcctattcatcctgctgacaatgaagcagt | Btnl2 |
| | cagtctggggtctgcccctagcatccacatggagggacctggggagagtggagtccagcttgtgtgc | (isoform 1) |
| | actgcaaggggctggttcccagagccccaggtgtattgggaagacatccggggagagaagctg | |
| | ctggccgtgtctgagcatcgcatccaagataaagatggcctgttctatgcggaagccaccctggtg | |
| | gtcaggaacgcctctgcagagtctgtgtcctgcttggtccacaaccccgtcctcactgaggagaa | |
| | ggggtcggtcatcagcctcccagagaaactccagactgagctggcttctttaaaagtgaatggac | |
| | cttcccagcccatcctcgtcagagtgggagaagatatacagctaacctgttacctgtcccccaagg | |
| | cgaatgcacagagcatggaggtgaggtgggaccgatcccaccgttaccctgctgtgcatgtgtat | |
| | atggatggggaccatgtggctggagagcagatggcagagtacagagggaggactgtactggtg | |
| | agtgacgccattgacgagggcagactgaccctgcagatactcagtgccagaccttcggacgacg | |
| | ggcagtaccgctgcctttttgaaaaagatgatgtctaccaggaggccagtttggatctgaaggtggt | |
| | aagtctgggttcttccccactgatcactgtggaggggcaagaagatggagaaatgcagccgatgt | |
| | gctcttcagatgggtggttcccacagccccacgtgccatggagggacatggaaggaaagacgat | |
| | accatcatcttcccaggccctgactcaaggcagccacgggctgttccacgtgcagacattgctaa | |
| | gggtcacaaacatctccgctgtggacgtcacttgttccatcagcatcccctttttgggcgaggagaa | |
| | aatcgcaactttttctctctcagagtccaggatgacgtttttgtggaaaacactgcttgtttgggga | |
| | ttgcttcttgctgtggctgtaggcctgcccaggaagaggagctgaaaagagtgaatgtgacattggc | |
| | ttcaaacacagctcacctgagactgatttcttctg | |
| 6 | ccgggtgtgtcacagcctcacttaggcacagcgtttccttgtctttgttccagggaggatggtggat | Human |
| | tttccaggctacaatctgtctggtgcagtcgcctccttcctattcatcctgctgacaatgaagcagt | Btnl2 |
| | cagagaaactccagactgagctggcttctttaaaagtgaatggaccttcccagcccatcctcgtcag | (isoform 2) |
| | agtgggagaagatatacagctaacctgttacctgtcccccaaggcgaatgcacagagcatggaggt | |
| | gaggtgggaccgatcccaccgttaccctgctgtgcatgtgtatatggatggggaccatgtggctg | |
| | gagagcagatggcagagtacagagggaggactgtactggtgagtgacgccattgacgagggca | |
| | gactgaccctgcagatactcagtgccagaccttcggacgacgggcagtaccgctgcctttttgaaa | |
| | aagatgatgtctaccaggaggccagtttggatctgaaggtggtaagtctgggttcttccccactgat | |
| | cactgtggaggggcaagaagatggagaaatgcagccgatgtgctcttcagatgggtggttccca | |
| | cagccccacgtgccatggagggacatggaaggaaagacgataccatcatcttcccaggccctga | |
| | ctcaaggcagccacgggctgttccacgtgcagacattgctaagggtcacaaacatctccgctgtgga | |
| | cgtcacttgttccatcagcatcccctttttgggcgaggagaaaatcgcaactttttctctctcagag | |
| | tccaggatgacgtttttgtggaaaacactgcttgtttggggattgcttcttgctgtggctgtaggcc | |
| | tgcccaggaagaggagctgaaaagagtgaatgtgacattggcttcaaacacagctcacctgagact | |
| | gatttcttctg | |

As used herein, the term “variant” refers to a first molecule that is related to a second molecule (also termed a “parent” molecule). The variant molecule can be derived from, isolated from, based on or homologous to the parent molecule. The variant molecule may include a fragment of the parent molecule. For example, the mutant forms of Btnl2, including the Btnl2 mutant with a cysteine substitution, are variants of the wild-type Btnl2. The term variant can be used to describe either polynucleotides or polypeptides.

As applied to proteins, a variant polypeptide can have an entire amino acid sequence identity with the original parent polypeptide or can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence. Polypeptide variants include polypeptides comprising the entire parent polypeptide, and further comprising additional fused amino acid sequences. Polypeptide variants also include polypeptides that are portions or subsequences of the parent polypeptide, for example, unique subsequences (e.g., as determined by standard sequence comparison and alignment techniques) of the polypeptides disclosed herein are also encompassed by the present disclosure.

In some embodiments, a variant of an amino acid sequence can be a second amino acid sequence that is at least 50% or more identical in amino acid sequence compared to the original amino acid sequence. In some embodiments, a variant of an amino acid sequence can be a second amino acid sequence that is at least 60% more identical in amino acid sequence compared to the original amino acid sequence. In some embodiments, a variant of an amino acid sequence can be a second amino acid sequence that is at least 70% or more identical in amino acid sequence compared to the original amino acid sequence. In some embodiments, a variant of an amino acid sequence can be a second amino acid sequence that is at least 80%, or more identical in amino acid sequence compared to the original amino acid sequence. In some embodiments, a variant of an amino acid sequence can be a second amino acid sequence that is at least 90% or more identical in amino acid sequence compared to the original amino acid sequence. In some embodiments, a variant of an amino acid sequence can be a second amino acid sequence that is at least 95% or more identical in amino acid sequence compared to the original amino acid sequence. In some embodiments, a variant of an amino acid sequence can be a second amino acid sequence that is at least 98% or more identical in amino acid sequence compared to the original amino acid sequence. In some embodiments, a variant of an amino acid sequence can be a second amino acid sequence that is at least 99% or more identical in amino acid sequence compared to the original amino acid sequence.

In another aspect, polypeptide variants include polypeptides that contain minor, trivial, or inconsequential changes to the parent amino acid sequence. For example, minor, trivial, or inconsequential changes include amino acid changes (including substitutions, deletions, and insertions) that have little or no impact on the biological activity of the polypeptide and yield functionally identical polypeptides, including additions of non-functional peptide sequences. In other aspects, the variant polypeptides of the present disclosure change the biological activity of the parent molecule. A person skilled in the art will appreciate that many variants of the disclosed polypeptides are encompassed by the present disclosure.

In some aspects, polynucleotide or polypeptide variants of the present disclosure can include variant molecules that alter, add or delete a small percentage of the nucleotide or amino acid positions, for example, typically less than about 10%, less than about 5%, less than 4%, less than 2% or less than 1%.

In some embodiments, polynucleotide or polypeptide variants of the present disclosure can include variant molecules that alter, add or delete less than about 10% of the nucleotide or amino acid positions. In some embodiments, polynucleotide or polypeptide variants of the present disclosure can include variant molecules that alter, add or delete less than about 5% of the nucleotide or amino acid positions. In some embodiments, polynucleotide or polypeptide variants of the present disclosure can include variant molecules that alter, add or delete less than about 4% of the nucleotide or amino acid positions. In some embodiments, polynucleotide or polypeptide variants of the present disclosure can include variant molecules that alter, add or delete less than about 2% of the nucleotide or amino acid positions. In some embodiments, polynucleotide or polypeptide variants of the present disclosure can include variant molecules that alter, add or delete less than about 1% of the nucleotide or amino acid positions.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

In some embodiments, the Btnl2 variant may include one or more conservative modifications. The Btnl2 variant with one or more conservative modifications may retain the desired functional properties, which can be tested using the functional assays known in the art. As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine). The Btnl2 protein with one or more conservative modifications may retain the desired functional properties, which can be tested using the functional assays known in the art.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988), which is hereby incorporated by reference in its entirety) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970), which is hereby incorporated by reference in its entirety) algorithm which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the XBLAST program (version 2.0) of Altschul et al. (1990)J. Mol. Biol. 215:403-10, which is hereby incorporated by reference in its entirety. BLAST protein searches can be performed with the XBLAST program, score=50, wordlength=3 to obtain amino acid sequences homologous to the antibody molecules of the present disclosure. To obtain gapped alignments for comparison purposes, Gapped BLAST can be utilized as described in Altschul et al. (1997) Nucleic Acids Res. 25 (17): 3389-3402, which is hereby incorporated by reference in its entirety. When utilizing BLAST and Gapped BLAST programs, the default parameters of the respective programs (e.g., XBLAST and NBLAST) can be used. (See www.ncbi.nlm.nih.gov).

In some embodiments, the Btnl2 variant can be conjugated or linked to a detectable tag or a detectable marker (e.g., a radionuclide, a fluorescent dye). In some embodiments, the detectable tag can be an affinity tag. The term “affinity tag” as used herein relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, 3×FLAG tag, a SUMO tag, and MBP-tag (MBP: maltose-binding protein). Further examples of affinity tags can be found in Kimple et al., Curr Protoc Protein Sci. 2013 Sep. 24; 73: Unit 9.9, which is hereby incorporated by reference in its entirety.

In some embodiments, the detectable tag can be conjugated or linked to the N- and/or C-terminus of the Btnl2 variant. The detectable tag and the affinity tag may also be separated by one or more amino acids. In some embodiments, the detectable tag can be conjugated or linked to the Btnl2 variant via a cleavable element. In the context of the present disclosure, the term “cleavable element” relates to peptide sequences that are susceptible to cleavage by chemical agents or enzyme means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “fusion polypeptide” or “fusion protein” means a protein created by joining two or more polypeptide sequences together. The fusion polypeptides encompassed in this disclosure include translation products of a chimeric gene construct that joins the nucleic acid sequences encoding a first polypeptide with the nucleic acid sequence encoding a second polypeptide to form a single open reading frame. In other words, a “fusion polypeptide” or “fusion protein” is a recombinant protein of two or more proteins which are joined by a peptide bond or via several peptides. The fusion protein may also comprise a peptide linker between the two domains.

In some embodiments, the Btnl2 agent comprises a fusion protein comprising a Btnl2 protein or variant thereof. In some embodiments, the fusion protein further comprises a tissue-specific signal. As used herein, the term “tissue-specific signal” refers to a molecule that is capable of targeting a Btnl2 agent to a specific tissue. For example, the tissue-specific signal can be a molecule that binds to a protein (e.g., mucosal vascular addressing cell adhesion molecule 1 (MADCAM1) expressed in a specific tissue (e.g., intestine). The tissue-specific signal can be a signal peptide or an antibody that binds specifically to (e.g., mucosal vascular addressing cell adhesion molecule 1 (MADCAM1) expressed in a specific tissue (e.g., intestine).

In some embodiments, the tissue-specific signal comprises a MADCAM1 inhibitor or an anti-MADCAM1 antibody. In some embodiments, the Btnl2 agent comprises a fusion protein comprising Btnl2 protein or a fragment thereof and an anti-MADCAM1 antibody such as anti-MAdCAM-1mAb MECA-367 (ATCC Accession No. HB-9478, including periodic GenBank sequence updates) or MECA-367 described in US20180140601A1, the relevant portion of which is hereby incorporated by reference in its entirety.

In some embodiments, the Btnl2 agent comprises a vector having a polynucleotide sequence encoding the Btnl2 protein or variant thereof or encoding a fusion protein comprising the Btnl2 protein or variant thereof. In some embodiments, the Btnl2 agent comprises a nucleic acid (e.g., DNA, RNA) having a polynucleotide sequence encoding the Btnl2 protein or variant thereof or encoding a fusion protein comprising the Btnl2 protein or variant thereof. In some embodiments, the polynucleotide sequence is RNA.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present disclosure comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the present disclosure comprises an expression cassette that comprises polynucleotide sequences that encode Btnl2 or variant thereof.

In some embodiments, the Btnl2 agent is a Btnl2 agent having agonist activity. The term “agonist” refers to a compound that causes agonism of the Btnl2 pathway and causes a response in a cell, e.g., suppression of the BTNL2 receptor-expressing immune cells. The Btnl2 agonist mimics the action of an endogenous ligand (Btnl2), resulting in a physiological response similar to that provided by the endogenous ligand. The term includes Btnl2 agents (e.g., a Btnl2 variant/fragment, a fusion protein comprising Btnl2, or a Btnl2 mimic) that, upon administration to a subject in need thereof, cause an upregulation and/or an increase in the activity of Btnl2-mediated signaling pathways. In some embodiments, the term includes a Btnl2 agent that leads to a reduction in the number or activity of T cells upon administration to a subject.

In some embodiments, the Btnl2 agent for increasing a level or activity of Btnl2 may be provided as a composition, e.g., a pharmaceutical composition. The composition may include one or a combination of the Btnl2 protein or variants thereof, formulated together with a pharmaceutically acceptable carrier. In some embodiments, such compositions may include one or a combination of (e.g., two or more different) Btnl2 variants. For example, the composition can include a combination of the Btnl2 variants having different genetic modifications.

As used herein, the term “composition” or “pharmaceutical composition” refers to a mixture of at least one component useful within the present disclosure with other components, such as carriers, stabilizers, diluents, dispersing agents, suspending agents, thickening agents, and/or excipients. The pharmaceutical composition facilitates administration of one or more components of the present disclosure to an organism.

Pharmaceutical compositions or therapeutic formulations of the Btnl2 agent for increasing a level or activity of Btnl2 (e.g., Btnl2 protein or variant thereof, Btnl2 agonist) can be prepared by mixing the Btnl2 agent thereof having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed and include buffers such as phosphate, citrate, and other organic acids; antioxidants including ascorbic acid and methionine; preservatives (e.g., octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONIC, or polyethylene glycol (PEG).

The formulation may also contain more than one active ingredients as necessary for the particular indication being treated, preferably those with complementary activities that do not adversely affect each other. For instance, the formulation may further comprise another anti-inflammation agent. Such molecules are suitably present in combination in amounts that are effective for the purpose intended.

The active ingredients may also be entrapped in microcapsule prepared, for example, by coacervation techniques or by interfacial polymerization, for example, hydroxymethyl cellulose or gelatin-microcapsule and poly-(methylmethacrylate) microcapsule, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the Btnl2 agent for increasing a level or activity of Btnl2 (e.g., the Btnl2 protein or variant thereof), which matrices are in the form of shaped articles, e.g., films, or microcapsule. Examples of sustained-releasable matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), and poly-d(−)-3-hydroxybutyric acid (PHB). While polymers such as ethylene-vinyl acetate and lactic acid-glycolic acid enable release of molecules for over 100 days, certain hydrogels release proteins for shorter time periods. When encapsulated, the Btnl2 agent remains in the body for a long time and may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiol-disulfide interchange, stabilization may be achieved by modifying sulfhydryl residues, lyophilizing from acidic solutions, controlling moisture content, using appropriate additives, and developing specific polymer matrix compositions.

The formulations to be used for in vivo administration must be sterile, which can be readily accomplished by filtration through sterile filtration membranes. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

c. Administration and Dosage Regimens

The Btnl2 agent for increasing a level or activity of Btnl2 (e.g., Btnl2 protein or variant thereof) can be administered as a single dose or more commonly can be administered on multiple occasions. Intervals between single dosages can be, for example, weekly, monthly, every three months or yearly. Intervals can also be irregular as indicated by measuring blood levels of Btnl2 protein or variant thereof in the patient.

The Btnl2 agent or the pharmaceutical composition thereof can be administered via one or more routes of administration using one or more of a variety of methods known in the art. As will be appreciated by the skilled artisan, the route and/or mode of administration will vary depending upon the desired results. For example, administration for the Btnl2 protein or variant thereof may include intravenous, intramuscular, intradermal, intraperitoneal, subcutaneous, spinal or other parenteral routes of administration, for example, by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion. Alternatively, a Btnl2 protein or variant thereof can be administered via a non-parenteral route, such as a topical, epidermal or mucosal route of administration, for example, intranasally, orally, vaginally, rectally, sublingually or topically.

In some embodiments, the Btnl2 agent for increasing a level or activity of Btnl2 (e.g., Btnl2 protein or variant thereof) or the pharmaceutical composition can be administered intratumorally, intravenously, subcutaneously, intraosseously, orally, transdermally, in sustained release, in controlled release, in delayed release, as a suppository, or sublingually.

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. The specification for the dosage unit forms of the present disclosure are dictated by and directly dependent on (a) the unique characteristics of the active compound and the particular therapeutic effect to be achieved, and (b) the limitations inherent in the art of compounding such an active ingredient for the treatment of sensitivity in individuals.

For administration of the Btnl2 agent for increasing a level or activity of Btnl2, the dosage ranges from about 0.0001 to 100 mg/kg (e.g., 0.001, 0.01, 0.1, 0.5, 1, 3, 5, 10, 15, 20, 25, 30, 35, 40, 45, 50, 55, 60, 65, 70, 75, 80, 85, 90, 95, 100 mg/kg), and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for a Btnl2 protein or variant thereof of the present disclosure include 1 mg/kg body weight or 3 mg/kg body weight via intravenous administration, with the Btnl2 protein or variant thereof being given using one of the following dosing schedules: (i) every four weeks for six dosages, then every three months; (ii) every three weeks; and (iii) 3 mg/kg body weight once followed by 1 mg/kg body weight every three weeks.

Alternatively, the Btnl2 agent can be administered as a sustained release formulation, in which case less frequent administration is required. Dosage and frequency vary depending on the half-life of the Btnl2 agent in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the compositions may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors, including the activity of the particular compositions of the present disclosure employed, the route of administration, the time of administration, the rate of excretion of the particular active ingredient being employed, the duration of the treatment, other drugs, compounds and/or materials used in combination with the particular compositions employed, the age, sex, weight, condition, general health and prior medical history of the patient being treated, and like factors well known in the medical arts.

As used herein, the terms “effective amount,” “effective dose,” and “effective dosage” refer to an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug/agent is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Pharmaceutical compositions disclosed herein may be administered with medical devices known in the art. For example, a therapeutic pharmaceutical composition of the present disclosure may be administered with a needleless hypodermic injection device, such as a device disclosed in U.S. Pat. Nos. 5,399,163, 5,383,851, 5,312,335, 5,064,413, 4,941,880, 4,790,824, and 4,596,556, which are hereby incorporated by reference by their entireties. Examples of well-known implants and modules useful in connection with the present disclosure include those described in U.S. Pat. Nos. 4,487,603, 4,486,194, 4,447,233, 4,447,224, 4,439,196, and 4,475,196, which are hereby incorporated by reference by their entireties. Many other such implants, delivery systems, and modules are suitable for use with the present disclosure and known to those skilled in the art.

In some embodiments, the method of treating an inflammatory disorder in a subject, as described herein, further comprises administering to the subject a second therapeutic agent or therapy. In some embodiments, the second therapeutic agent is an anti-inflammation agent.

As used herein, the terms “co-administration” and “co-administered” refer to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In some embodiments, the second therapeutic agent, e.g., the anti-inflammation agent, is administered to the subject before, after, or concurrently with the second therapeutic agent.

d. Methods of Eliciting Antimicrobial Responses

In another aspect, this disclosure further provides a method of eliciting an antimicrobial response in a subject, comprising administering to the subject an effective amount of a Btnl2 agent, the Btnl2 agent capable of increasing a level or activity of Btnl2 in the subject. The antimicrobial response may kill or slow the growth of microorganisms, such as bacteria, viruses, protozoans, and fungi.

In some embodiments, the IELs comprise γδ T cells. In some embodiments, the IELs comprise intestinal γδ intraepithelial lymphocytes (IELs). In some embodiments, the intestinal γδ IELs comprise jejunal γδ IELs, ileal γδ IELs, colonic γδ IELs, or combinations thereof. In some embodiments, the intestinal γδ IELs comprise the ileal γδ IELs. In some embodiments, the intestinal γδ IELs comprise ileal CD8αα+γδ IELs.

In some embodiments, the Btnl2 agent comprises a Btnl2 protein or variant thereof or a fusion protein comprising the Btnl2 protein or variant thereof. In some embodiments, the Btnl2 protein comprises an amino acid sequence having at least 90% (e.g., 90%, 92%, 94%, 95%, 96%, 97%, 98%, 99%) identity to an amino acid sequence of SEQ ID NOs: 1-3 or comprises an amino acid sequence of SEQ ID NOs: 1-3.

In some embodiments, the fusion protein further comprises a tissue-specific signal. In some embodiments, the tissue-specific signal comprises a MADCAM1 inhibitor or an anti-MADCAM1 antibody. In some embodiments, the fusion protein comprises the Btnl2 protein or variant thereof and an anti-MADCAM1 antibody.

In some embodiments, the Btnl2 agent comprises a vector having a polynucleotide sequence encoding the Btnl2 protein or variant thereof or encoding a fusion protein comprising the Btnl2 protein or variant thereof.

In some embodiments, the Btnl2 agent comprises a nucleic acid (e.g., DNA or RNA) having a polynucleotide sequence encoding the Btnl2 protein or variant thereof or encoding a fusion protein comprising the Btnl2 protein or variant thereof. In some embodiments, the polynucleotide sequence is RNA.

e. Methods of Identifying Btnl2 Agonists

In yet another aspect, this disclosure provides a method for identifying a Btnl2 agent capable of reducing IELs in a subject, comprising: (a) administering to the subject an amount of a Btnl2 agent capable of increasing a level or activity of Btnl2 or having Btnl2 agonist activity; (b) performing an assay on a sample obtained from the subject and determining the number of the IELs in the sample; and (c) identifying the Btnl2 agent as having capability of reducing the IELs in the subject if the subject has an increased number of the IELs as compared to a predetermined reference value.

In some embodiments, the lymphocytes comprise CD4+ T cells, CD8+T cells, regulatory T cells (Treg) or tissue-specific T cells. In some embodiments, the IELs comprise intestinal γδ IELs. In some embodiments, the intestinal γδ IELs comprise duodenal γδ IELs, jejunal γδ IELs, ileal γδ IELs, or combinations thereof.

The level or activity of Btnl2 may be measured by determining or estimating a protein level or mRNA level. Methods for determining or estimating a protein level or mRNA level are well known in the art. For example, the protein level (e.g., protein expression level) of Btnl2 can be determined by SDS-PAGE, Western blot, or an immunoassay (e.g., immunoblotting assay, immunoprecipitation assay). The mRNA level may be determined by RT-PCR.

Measuring the levels of Btnl2 can be performed by assaying the proteins themselves (by Western blotting, ELISA, RIA, and other techniques known to one skilled in the art), by assaying the mRNA encoding these proteins (such as quantitative PCR, Northern blotting, RNAse protection assay, RNA dot-blotting, and other techniques known to one skilled in the art), or by assaying the activity of the regulatory elements of the genes for Btnl2. For example, the activity of regulatory elements can be assessed by reporter constructs consisting of DNA segments from the promoter, enhancer, and/or intronic elements coupled to cDNAs encoding reporters (such as luciferase, beta-galactosidase, green fluorescent protein, or other reporting genes that can be easily assayed). These reporter constructs can be transfected into cells, either stably or transiently.

In some embodiments, the method may include culturing or expanding a test cell and/or a control cell. The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” may be used interchangeably herein, and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

The terms “polypeptide,” “peptide” and “protein” may be used interchangeably herein, and refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “operably linked” refers to a functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions in the same reading frame.

The term “linker” refers to any means, entity, or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea, and the like. To provide for linking, the domains can be modified by oxidation, hydroxylation, substitution, reduction, etc., to provide a site for coupling. Methods for conjugation are well known by persons skilled in the art and are encompassed for use in the present disclosure. Linker moieties include, but are not limited to, chemical linker moieties, or for example, a peptide linker moiety (a linker sequence).

By “isolated” nucleic acid molecule or polynucleotide is intended as a nucleic acid molecule, DNA or RNA, which has been removed from its native environment. For example, a recombinant polynucleotide encoding a therapeutic polypeptide contained in a vector is considered isolated for the purposes of the present disclosure. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present disclosure, as well as positive and negative strand forms and double-stranded forms. Isolated polynucleotides or nucleic acids, according to the present disclosure, further include such molecules produced synthetically. In addition, a polynucleotide or a nucleic acid may be or may include a regulatory element such as a promoter, ribosome binding site, or a transcription terminator.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, or at least 95%, 98% or 99% sequence identity. In some embodiments, the term means two peptide sequences, when optimally aligned, such as by the programs GAP or BESTFIT using default gap weights, share at least 95% sequence identity. In some embodiments, the term means two peptide sequences, when optimally aligned, such as by the programs GAP or BESTFIT using default gap weights, share at least 98% sequence identity. In some embodiments, the term means two peptide sequences, when optimally aligned, such as by the programs GAP or BESTFIT using default gap weights, share at least 99% sequence identity. In some embodiments, residue positions, which are not identical, differ by conservative amino acid substitutions.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is hereby incorporated by reference in its entirety.

As used herein, “homology” refers to the level of similarity between two or more nucleic acid and/or amino acid sequences in terms of percent of positional identity (i.e., sequence similarity or identity). Homology also refers to the concept of similar functional properties among different nucleic acids or proteins. A homologue of a nucleotide sequence or polypeptide as disclosed herein may have a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to the nucleotide sequence or polypeptide as disclosed herein.

As used herein, “genotyping” refers to a process of determining the specific allelic composition of a cell and/or subject at one or more positions within the genome, e.g., by determining the nucleic acid sequence at that position. Genotyping refers to a nucleic acid analysis and/or analysis at the nucleic acid level, such as sequencing.

As used herein, “sequencing” refers to the determination of the exact order of nucleotide bases in a strand of DNA (deoxyribonucleic acid) or RNA (ribonucleic acid) or the exact order of amino acids residues or peptides in a protein. Nucleic acid sequencing can be done using Sanger sequencing or next-generation high-throughput sequencing.

As used herein, “next-generation sequencing” refers to oligonucleotide sequencing technologies that have the capacity to sequence oligonucleotides at speeds above those possible with conventional sequencing methods (e.g., Sanger sequencing), due to performing and reading out thousands to millions of sequencing reactions in parallel. Non-limiting examples of next-generation sequencing methods/platforms include Massively Parallel Signature Sequencing (Lynx Therapeutics); 454 pyro-sequencing (454 Life Sciences/Roche Diagnostics); solid-phase, reversible dye-terminator sequencing (Solexa/Illumina): SOLID technology (Applied Biosystems); Ion semiconductor sequencing (ION Torrent); DNA nanoball sequencing (Complete Genomics); and technologies available from Pacific Biosciences, Intelligen Bio-systems, Oxford Nanopore Technologies, and Helicos Biosciences. Next-generation sequencing technologies and the constraints and design parameters of associated sequencing primers are well known in the art (see, e.g., Shendure et al., “Next-generation DNA sequencing,” Nature, 2008, vol. 26, No. 10, 1135-1145; Mardis, “The impact of next-generation sequencing technology on genetics,” Trends in Genetics, 2007, 24 (3): 133-41; Su et al., “Next-generation sequencing and its applications in molecular diagnostics” Expert Rev Mol Diagn, 2011, 11 (3): 333-43; Zhang et al., “The impact of next-generation sequencing on genomics,” J Genet Genomics, 2011, 38 (3): 95-109; (Nyren et al. Anal Biochem 208:17175 (1993); Bentley, D. R. Curr Opin Genet Dev, 16:545-52 (2006); Strausberg et al. Drug Disc Today, 13:569-77 (2008); U.S. Pat. Nos. 7,282,337; 7,279,563; 7,226,720; 7,220,549; 7,169,560; 6,818,395; 6,911,345; US Pub. Nos. 2006/0252077; 2007/0070349; 20070070349). All the references as mentioned are hereby incorporated by reference in their entireties.

The terms “disease,” “disorder,” and “condition” (as in medical condition) may be used interchangeably herein, and refer to an abnormal condition (e.g., inflammatory disorder) of the human or animal body or of one of its parts that impairs normal functioning, is typically manifested by distinguishing signs and symptoms, and causes the human or animal to have a reduced duration or quality of life.

As used herein, “inflammatory bowel disease” refers to a condition relating to abnormality of inflammation in the bowel. Inflammatory bowel diseases that can be treated according to the invention include but are not limited to Crohn's disease as may be enteritis, enterocolitis, Crohn's disease, granulomatous colitis, ileitis or Crohn's colitis, and ulcerative colitis as may be ulcerative colitis, sigmoiditis, pancolitis, and ulcerative proctitis.

As used herein, “modulate” refers to any change in biological state, i.e., increasing, decreasing, and the like.

As used herein, “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

As used herein, the term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of the disclosure, and are physiologically acceptable to the subject. Supplementary active compounds may also be incorporated into the compositions.

As used herein, “in vitro” refers to events that occur in an artificial environment, e.g., in a test tube or reaction vessel, in cell culture, etc., rather than within a multi-cellular organism.

As used herein, “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

As used herein, “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like do not necessarily refer to the same embodiment, but may unless the context dictates otherwise.

As used herein, the terms “and/or” or “/” means any one of the items, any combination of the items, or all of the items with which this term is associated.

As used herein, the term “substantially” does not exclude “completely,” e.g., a composition which is “substantially free” from Y may be completely free from Y. Where necessary, the word “substantially” may be omitted from the definition of the present disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The use of any and all examples, or exemplary language (e.g., “such as”) provided herein, is intended merely to better illuminate the invention and does not pose a limitation on the scope of the present disclosure unless otherwise claimed. No language in the specification should be construed as indicating any non-claimed element as essential to the practice of the present disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

The following examples are put forth so as to provide those of ordinary skill in the art with a complete disclosure and description of how to make and use the methods and compositions of the present disclosure and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1

This example describes the materials and methods used in EXAMPLES 2-12.

### Mice

Eight to twelve-week-old female C57BL/6 mice were obtained from Jackson Laboratory. Btnl2-KO mice on a C57BL/6 background were generated and maintained at Regeneron Pharmaceuticals Inc. using the VelociGene technology (Poueymirou et al., Nat Biotechnol, 2007, 25 (1): 91-99; Valenzuela et al., Nat Biotechnol, 2003, 21 (6): 652-59). A LacZ cassette was inserted in-frame with the start codon followed by a selection cassette that disrupted the transcription of the Btnl2 gene resulting in a null allele. Heterozygous mice were interbred to produce homozygous KO and WT littermates. Btnl2 expression pattern was confirmed by β-galactosidase staining, and Btnl2 targeted deletion was measured by quantitative RT-PCR and RNA sequencing of the small intestine. Btnl2-KO and WT female mice were used at 10-17 weeks of age for all the experiments except when otherwise indicated. Female littermates were cohoused after weaning for several weeks and assigned randomly to experimental groups in disease settings. All animals were maintained under pathogen-free conditions, and experiments were performed according to protocols approved by the Institutional Animal Care and Use Committee at Regeneron Pharmaceuticals Inc.

### Isolation of Intestinal Epithelial Cells (IECs), Intraepithelial Lymphocytes (IELs), and Lamina Propria Lymphocytes (LPLs)

The small intestine was divided into three equal segments, and lymphocyte isolation proceeded as described previously (Ivanov et al., Cell, 2006. 126 (6): 1121-33). To isolate IEC and IEL fractions, small intestine was cut into 2 cm pieces and incubated in HBSS containing 5 mM EDTA, 10 mM HEPES, and 2% fetal calf serum (FCS) twice for 15 min at 37° C. with shaking at 150 rpm. After vigorous vortexing, the intestinal pieces were washed over 100 μm cell strainer and centrifuged on a 40%/80% Percoll gradient (GE Healthcare) at 2500 rpm for 20 min at 20° C. The top layer containing IECs was collected, washed, and resuspended in Trizol for RNA extraction. IEL fraction was collected from the interface, washed, and resuspended in Miltenyi MACS buffer. Following IEL isolation, LPLs were isolated from intestinal pieces by incubation in HBSS w/o Ca^{2 +}/Mg²⁺ supplemented with 50 U/mL Collagenase D (Roche), 0.25 mg/mL DNase I (Sigma-Aldrich), 50 U/mL Dispase (Corning), and 5% FCS for two rounds of 25 min at 37° C. with shaking at 150 rpm. Cells were centrifuged on a 40%/80% Percoll gradient (GE Healthcare), and LPLs were collected from the interface, washed, and resuspended in MACS buffer for immediate surface cell staining.

### Mesenteric Lymph Node and Peyer's Patch Immunophenotyping

Peyer's Patches were collected from the whole small intestine, washed with ice-cold DPBS, and incubated with 50 U/mL Collagenase D (Roche), 0.25 mg/mL DNase I (Sigma-Aldrich), 50 U/mL Dispase (Corning), and 5% FCS for 25 min at 37° C. with shaking at 150 rpm. Mesenteric lymph nodes were minced in HBSS with Ca²⁺/Mg²⁺ containing 15 U/mL Collagenase D (Roche) and 50 μg/mL DNase I (Sigma-Aldrich) and incubated for 20 min at 37° C. without shaking. Cells were resuspended in MACS buffer for immediate surface staining.

### Flow Cytometry

Flow cytometry antibodies were purchased from Biolegend (US), BD Biosciences (US), TONBO Biosciences (US), eBioscience (US) and ThermoFischer (US). Dead cells were excluded using LIVE/DEAD fixable blue dead cell stain (Thermo Fischer Scientific, Cat #L23105). Fc receptors were blocked using purified anti-mouse CD16/32 (BD Pharmigen, Clone 2.4G2, Cat #553142) and 2% each of normal mouse serum (Jackson ImmunoResearch, Cat #015-000-120), rat serum (Jackson ImmunoResearch, Cat #012-000-120) and hamster serum (Jackson ImmunoResearch, Cat #007-000-120). The following antibodies were used for the staining according to manufacturer's instructions: CD45-BV510 (Biolegend, Clone #30-F11, Cat #103138), CD8α-AF700 (Biolegend, Clone #53-6.7, Cat #100730), CD8β-PerCP/Cy5.5 (Biolegend, Clone #YTS156.7.7, Cat #126610), TCRβ-BV711 (Biolegend, Clone #H57-597, Cat #109243), TCRβ-APC/Cy7 (Biolegend, Clone #H57-597, Cat #109220), TCRγ/8-PE/Cy7 (Biolegend, Clone #GL3, Cat #118124), CD11b-APC/Cy7 (Biolegend, Clone #M1/70, Cat #101226), CD11c-APC/Cy7 (Biolegend, Clone #N418, Cat #117324), CD11c-AF700 (Biolegend, Clone #N418, Cat #117320), CD11c-PE/Cy7 (Biolegend, Clone #N418, Cat #117318), Gr1-APC/Cy7 (Biolegend, Clone #RB6-8C5, Cat #108424), B220-APC/Cy7 (Biolegend, Clone #RA3-6B2, Cat #103224), B220-AF700 (Biolegend, Clone #RA3-6B2, Cat #103232), B220-BV650 (Biolegend, Clone #RA3-6B2, Cat #103241), NK1.1-APC/Cy7 (Biolegend, Clone #PK136, Cat #108724), MHCII-BV421 (Biolegend, Clone #M5/114.15.2, Cat #107632), Ly6C-PerCP/Cy5.5 (Biolegend, Clone #HK1.4, Cat #128012), CD64-PE (Biolegend, Clone #X54-5/7.1, Cat #139304), CD103-FITC (Biolegend, Clone #2E7, Cat #121420), CX3CR1-Biotin (Biolegend, Clone #SA011F11, Cat #149018), Streptavidin-PE/Dazzle 594 (Biolegend, Cat #405248), NKp46-PE/Dazzle594 (Biolegend, Clone #29A1.4, Cat #137630), CD4-PerCP/Cy5.5 (Biolegend, Clone #GK1.5, Cat #100434), CD4-VF450 (Tonbo Biosciences, Clone #GK1.5, Cat #75-0041-U100), c-KIT-PE-Cy7 (Biolegend, Clone #ACK2, Cat #135112), CD44-APC/Cy7 (Biolegend, Clone #IM7, Cat #103028), CD44-BV650 (Biolegend, Clone #IM7, Cat #103049), RORγt-APC (eBiosciences, Clone #AFKJS-9, Cat #17-6988-82), FoxP3-AF700 (eBioscience, Clone #FJK-16s, Cat #56-5773-82), FoxP3-eF450 (eBioscience, Clone #FJK-16s, Cat #48-5773-82), LegendScreen Mouse PE kit (Biolegend, Cat #700005). For intranuclear staining, cells were incubated with fixable viability dye and surface markers prior to fixation and permeabilization using the FoxP3/Transcription factor fixation and permeabilization kit (eBioscience) according to manufacturer's instructions.

BrdU (Sigma-Aldrich) incorporation was assessed 3 days after continuous administration in drinking water dissolved at 0.8 mg mL⁻¹ in 3% sucrose. Briefly, IELs were fixed with BD Cytofix/Cytoperma for 20 min at 20° C., washed, and incubated with 1× DPBS supplemented with Ca²⁺/Mg²⁺, 10% FCS, and 10% DMSO for 10 min at 20° C. Cells were re-fixed with BD Cytofix/Cytoperma for 5 min at 20° C., washed, and incubated with 0.5 mg mL⁻¹ DNAse I (Sigma-Aldrich) for 1 hr at 37° C. Cells were stained with BrdU-AF647 (MoBU-1) (Thermo Fisher Scientific, Clone #MoBU-1, Cat #B35133) at 20° C., washed, and resuspended for acquisition.

Flow cytometry was performed on LSRFortessa X-20 instrument (BD Biosciences), data were analyzed using FlowJo software (BD Biosciences) and plotted using GraphPad Prism™ (GraphPad Software, Inc.).

### In Vitro IEL Proliferation Assay

96-well flat −bottom plates were coated overnight with 1 mg mL⁻¹ purified anti-mouse CD3e (Tonbo Biosciences, 145-2C11, Cat #70-0031-M001) and 60 pmoles of mouse Btnl2-Fc, PDL1-Fc or mFc (Adipogen Life Sciences) at 4° C., and washed twice with DPBS before adding IELs to the cultures. Freshly isolated IELs were labeled with CellTrace CFSE Cell Proliferation dye according to the manufacturer's instructions (Thermo Fischer Scientific, Cat #C34554). CFSE-labeled IELs were plated at 200,000 cells per well in RPMI 1640 supplemented with 10% FCS, 1% Pen/Strep, 2% HEPES, 1% Glutamine, 1% non-essential amino acids, 1% sodium pyruvate, 0.1% b-mercapto-ethanol (Gibco), recombinant mouse IL-7 (10 ng mL⁻¹, R&D), recombinant mouse IL-15 (10 ng mL⁻¹, R&D) and recombinant human IL-2 (10 ng mL⁻¹, Peprotech). Cells were incubated for 72-96 hours at 37° C. in 5% CO₂ prior to analysis.

### In Vitro CD4+T Cell Proliferation Assay

96-well flat −bottom plates were coated overnight with 1 mg mL⁻¹ purified anti-mouse CD3e (Tonbo Biosciences, 145-2C11, Cat #70-0031-M001), 1 mg mL⁻¹ purified anti-mouse CD28 (Tonbo Biosciences, 37.51, Cat #70-0281-U500) and 60 pmoles of mouse Btnl2-Fc, PDL1-Fc, PDL2-Fc or mFc (Adipogen Life Sciences) at 4° C., and washed twice with DPBS before adding CD4⁺ T cells to the cultures. CD4⁺ T cells were enriched from pooled spleen and lymph nodes using mouse CD4 (L3T4) microbeads (Miltenyi Biotec) and labeled with CellTrace CFSE Cell Proliferation dye (Thermo Fischer Scientific, Cat #C34554). CFSE-labeled CD4⁺ T cells were plated at 80,000-100,000 cells per well in RPMI 1640 supplemented with 10% FCS, Pen/Strep, 2% HEPES, 1% Glutamine, 1% non-essential amino acids, 1% sodium pyruvate and 0.1% b-mercapto-ethanol (Gibco). Cells were incubated for 72 hours at 37° C. in 5% CO₂ prior to analysis.

### In Vitro CD4+T Helper Cell Differentiation Assay

96-well flat −bottom plates were coated overnight with 1 μg/mL purified anti-mouse CD38 (145-2C11, TONBO BIOSCIENCES), 1 μg/mL purified anti-mouse CD28 (37.51, TONBO BIOSCIENCES) and 60 pmoles of mouse Btnl2-Fc, PDL1-Fc, CD86-Fc, CTLA4-Fc or mFc (Adipogen Life Sciences) at 4° C., and washed twice with DPBS before adding naïve CD4⁺T cells to the cultures. Naïve CD4+ T cells were enriched from pooled spleen and lymph nodes using mouse naïve CD4⁺ T cell isolation kit (MILTENYI BIOTEC). Enriched naïve CD4+ T cells were plated at 80,000-100,000 cells per well in RPMI 1640 supplemented with 30 U/mL recombinant mouse IL-2 (PEPROTECH), 10% FCS, Pen/Strep, 2% HEPES, 1% Glutamine, 1% non-essential amino acids, 1% sodium pyruvate, and 0.1% β-mercapto-ethanol (GIBCO). For regulatory T cell differentiation, naïve CD4⁺ T cells cultures were further supplemented with 10 ng/ml recombinant mouse TGFβ1 (BIOLEGEND), 10 μg/mL anti-mouse IL-4 (11B11, BIOLEGEND), and 10 μg/mL anti-mouse IFNγ (XMG1.2, BIOLEGEND). For Th17 cell differentiation, naïve CD4+T cells cultures were further supplemented with 0.5 ng/mL recombinant mouse TGFβ1 (BIOLEGEND), 50 ng/mL recombinant mouse IL-6 (BIOLEGEND), 20 ng/ml recombinant mouse IL-23 (BIOLEGEND), 10 μg/mL anti-mouse IL-4 (11B11, BIOLEGEND), 10 μg/mL anti-mouse IFNγ (XMG1.2, BIOLEGEND), and in some cases, 20 ng/ml recombinant mouse IL-1B (R&D). Cells were incubated for 5 days at 37° C. in 5% CO₂ prior to analysis.

### Phospho-Flow Cytometry

Flow cytometry antibodies for staining of phosphorylated cell signaling molecules were purchased from CELL SIGNALING TECHNOLOGY (US) and BD BIOSCIENCES (US). Dead cells were excluded using LIVE/DEAD fixable blue dead cell stain (THERMO FISCHER SCIENTIFIC). Fc receptors were blocked using purified anti-mouse CD16/32 (93) and 2% each of normal mouse serum, rat serum, and hamster serum (JACKSON IMMUNORESEARCH). The following antibodies were used for staining according to manufacturer's instructions: phospho-Akt-Alexa Flour 647 (Ser473, D9E), phospho-S6-Alexa Fluor 647 (Ser235/236, D57.2.2E), phospho-FoxO1 (Thr24)/FoxO3a (Thr32)/FoxO4 (Th28) (4G6), FoxO1-PE (C29H4), STAT5-PE-Cy7 (pY694, 47), STAT1-BV421 (pY701, 4a), goat anti-rabbit IgG (H+L), F (ab′)₂ fragment-Alexa Fluor 647.

For Akt/S6/FoxO staining, after 15 hours of culture in Th17 and regulatory T cell differentiation conditions in the presence of Fc fusion proteins, CD4⁺ T cells were incubated with fixable viability dye at room temperature for 10 min, fixed in 2% paraformaldehyde (ELECTRON MICROSCOPY SCIENCES) on ice for 20 min, and then permeabilized in 90% methanol at −20° C. overnight. After washing in ice-cold 1× DPBS, cells were then incubated with Fc receptor blockers for 10 min, stained with Akt/S6/FoxO antibodies for 5 hours at 4° C., washed, and resuspended for acquisition.

For STAT staining, after 5 days of culture in regulatory T cell differentiation conditions in the presence of Fc fusion proteins, CD4⁺T cells were washed several times, resuspended in RPMI 1640 supplemented with 0.5% Bovine Serum Albumin (VWR), and rested on ice for 3 hours. Next, an equal volume of media containing fixable viability dye and lineage-skewing cytokines was added, and cells were incubated at 37° C. for 20 min. Immediately, cells were placed on ice and fixed in an equal volume of ice-cold 4% paraformaldehyde for 20 min. Cells were then washed in ice-cold 1× DPBS and permeabilized in 90% methanol at −20° C. overnight. After washing in ice-cold 1× DPBS, cells were incubated with Fc receptor blockers for 10 min, stained with STAT1/5 antibodies at 4° C. overnight, washed, and resuspended for acquisition.

### DSS-Induced Model of Chronic Colitis

Fourteen-twenty-week-old cohoused female Btnl2-KO and WT mice with an average body weight greater than 23 g were given 3% DSS (Sigma-Aldrich) in drinking water for 6-7 days, followed by distilled water for up to 10 days. Control group received only distilled water for the duration of the study. Mice were weighed and monitored daily for clinical signs of colitis (e.g., stool consistency, fecal blood). On day 15, mice were euthanized, and colon length was measured.

### Generation of Colon Homogenates and Measurement of Cytokines and Myeloperoxidase (MPO) Activity

6 mm pieces of distal colon or terminal ileum were placed in T-per buffer (Thermo Fisher Scientific) containing 1× Halt Protease Inhibitor Cocktail (Thermo Fisher Scientific), 0.5 M EDTA solution (Thermo Fisher Scientific), and two 3 mm tungsten carbide beads (Qiagen). Tissues were disrupted in TissueLyser II (Qiagen) for 10 min at an oscillation frequency of 27.5 Hz. Generated tissue homogenates were centrifuged at 15000 rcf for 10 min at 4° C., and the supernatants were collected into deep 96-well plates. Protein assay dye (BioRad) was used to quantify total protein content using Bradford protein assay according to manufacturer's instructions. Cytokine concentrations were measured using V-PLEX Plus Proinflammatory Panel 1 mouse kit according to manufacturer's instructions (Meso Scale Diagnostics). Absorbance was measured on Meso SECTOR S600 instrument (Meso Scale Diagnostics). Myeloperoxidase (MPO) activity was measured using a mouse MPO ELISA kit according to manufacturer's instructions (Hycult Biotech). Absorbance was measured on SpectraMax i3x instrument (Molecular Devices). Data analysis was performed using GraphPad Prismä (GraphPad Software, Inc.). Cytokine and MPO levels were normalized to total protein content.

### Histology

3 cm pieces of duodenum, jejunum, ileum, and colon were prepared as swiss rolls, fixed in 10% buffered formalin, embedded in paraffin, sectioned at 5 mm, and H&E stained. Histology was performed by HistoWiz Inc. (histowiz.com) using a Standard Operating Procedure and fully automated workflow. After staining, sections were dehydrated and filmed coverslipped using a TissueTek-Prisma and Coverslipper (Sakura). Whole slide scanning (40×) was performed on an Aperio AT2 (Leica Biosystems). Histopathological scoring was performed by an evaluator blinded to genotype, group assignment, and experimental outcome. The following features were evaluated for DSS-induced injury and scored based on previously published criteria (Izcue et al., Immunity, 2008. 28 (4): p. 559-70): degree of inflammation in lamina propria, goblet cell loss, abnormal crypts, presence of crypt abscesses, mucosal erosion and ulceration, submucosal spread to transmural involvement, number of neutrophils. Each parameter received a score from 0 to 4 with the maximum cumulative score of 17. Mucosal lesions in unchallenged mice were scored as described previously (Xiao et al., Sci Rep, 2017. 7: p. 40317): 0, normal; 1, mild sloughing of epithelial cells; 2, moderate sloughing of epithelial cells; 3, severe mucosal edema; 4, extensive mucosal injury. Data analysis was performed using GraphPad Prism™

### Quantitative PCR

RNA was isolated from IECs derived from duodenum, jejunum, ileum, and colon from cohoused unchallenged Btnl2-KO and WT mice. RNA was extracted from distal colon and terminal ileum from cohoused, water- and DSS-treated Btnl2-KO and WT mice. RNA was purified on Kingfisher flex (Thermo Fisher Scientific) using the MagMAX-96 for Microarrays Total RNA isolation kit (Thermo Fisher Scientific) with an additional DNAse I (Sigma-Aldrich) step added between the first and second washes. cDNA synthesis was performed using SuperScript VILO Master Mix (Thermo Fisher Scientific) according to manufacturer's instructions. qPCR was performed using MyTaq Mix (Bioline) and assay mix (Thermo Fisher Scientific or LGC BioSearch). qPCR was run on an ABI 7900HT Fast Real-Time PCR System with a 384-well block module and automation accessory (Thermo Fisher Scientific). Gene expression was normalized to β2m, and differences were determined using the 2ΔC(t) calculation.

TABLE 2
Example primer sequences

| | | SEQ | | SEQ | | SEQ |
|---|---|---|---|---|---|---|
| | | ID | | ID | | ID |
| Gene | Forward | NO | Reverse | NO | Probe | NO |
| BTNL2 | ggattgcccac | 7 | aggaccgac | 8 | tatctggcgtgg | 9 |
| | ggtatagtc | | cactctgaa | | ctgcctcctt | |
| | | | g | | | |
| BTNL1 | ggtgcagatgc | 10 | gccacactt | 11 | caggacccagat | 12 |
| | cggaatacag | | cccatgtca | | ggtgagacaagC | |
| | | | atg | | | |
| BTNL6 | gaggccatctt | 13 | ccaccgtct | 14 | tggcagcaatgg | 15 |
| | ggaactgaa | | tctggacct | | gctctgtcc | |
| | | | tt | | | |
| ß2m | gggaagccgaa | 16 | cccgttctt | 17 | acgtaacacagt | 18 |
| | catactgaact | | cagcatttg | | tccacccgcct | |
| | g | | gatttc | | | |
| Gzma | ggcgctttgatt | 19 | tgttctggc | 20 | tgactgctgccc | 21 |
| | gaaaagaactg | | tccttattg | | actgtaacgtgg | |
| | | | attgag | | | |

### Single-Cell RNA Sequencing of γδ IELs

Following IEL isolation, cells were stained with LIVE/DEAD fixable blue dead cell stain as per manufacturer's instructions (THERMO FISHER SCIENTIFIC). Fc receptors were blocked using purified anti-mouse CD16/32 (93) and 2% each of normal mouse serum, rat serum, and hamster serum (JACKSON IMMUNO RESEARCH), and cells were stained using the following antibodies: CD45-BV510 (30-F11), CD8β-AF700 (53-6.7), CD8β-PerCP/Cy5.5 (YTS156.7.7), TCRβ-BV711 (H57-597), TCRγδ-PE/Cy7 (GL3). Two mice were pooled per each sample.

γδ IELs were sorted from each sample using MoFlo Astrios EQ (BECKMAN COULTER). Two thirds of each sample were resuspended in RNA Lysis Buffer (ZYMO RESEARCH) and processed for bulk RNA sequencing. One third of each sample was pooled per segment of the small intestine per genotype, resuspended in PBS with 0.04% BSA, and loaded on a Chromium Single Cell Instrument (10× Genomics). RNAseq and V(D)J libraries were prepared using Chromium Single Cell 5′ Library, Gel Beads & Multiplex Kit (10× Genomics). After amplification, cDNA was divided into RNAseq and V(D)J library aliquots. To enrich the V(D)J library aliquot for γδ TCRs, cDNA was divided into two 10 ng aliquots and amplified in two rounds using internally designed primers. In particular, the following primers were used for the first round of amplification: MP147 for short R1 (ACACTCTTTCCCTACACGACGC) (SEQ ID NO: 22), MP371 for mouse TRGC1-3 (/5Biosg/TTCCTGGGAGTCCAGGATRGTATTG) (SEQ ID NO: 23), MP 372 for mouse TRGC4 (/5Biosg/CACCCTTATGACTTCAGGAAAGAACTTT) (SEQ ID NO: 24) and MP369 for mouse TRDC (/5Biosg/TTCCACAATCTTCTTGGATGATCTGAG) (SEQ ID NO: 25). For the second round of amplification, 20 ng aliquots from the first round were further amplified using MP147 for short R1 (ACACTCTTTCCCTACACGACGC) (SEQ ID NO: 26), MP373 a nested R2 plus mouse TRGC (GTGACTGGAGTTCAGACGTGTGCTCTTCCGATCTGTCCCAGYCTTATGGAGAT TTGT) (SEQ ID NO: 27), and MP370 a nested R2 plus mouse TRDC (GTGACTGGAGTTCAGACGTGTGCTCTTCCGATCTTAGTCACCTCTTTAGGGTAGAAA TCTT) (SEQ ID NO: 28).

V(D)J libraries were prepared from 25 ng of each mTRGC and mTRDC amplified cDNA. Paired-end sequencing was performed on Illumina NextSeq500 for RNAseq libraries (Read 1:26-bp for unique molecular identifier (UMI) and cell barcode, 8-bp i7 sample index, 0-bp i5, and Read 2:55-bp transcript read) and V(D)J libraries (Read 1:150-bp, 8-bp i7 sample index, 0-bp i5, and Read 2:150-bp read). For RNAseq libraries, Cell Ranger Single-Cell Software Suite (10× Genomics, v2.2.0) was used to perform sample de-multiplexing, alignment, filtering, and UMI counting. The mouse mm 10 genome assembly and RefSeq gene model for mouse were used for the alignment. For V(D)J libraries, Cell Ranger Single-Cell Software Suite (10× Genomics, v2.2.0) was used to perform sample de-multiplexing, de novo assembly of read pairs into contigs, align and annotate contigs against all the germline segment V(D)J reference sequences from IMGT, label and locate CDR3 regions, group clonotypes.

### Single-Cell RNA Sequencing Data Analysis

scRNAseq data were analyzed using the Seurat R package (Butler et al., Nature Biotechnology, 2018. 36:411.). Cells with fewer than 500 genes or more than 10% of mitochondrial RNA content were excluded during the quality control (QC) step. The remaining cells underwent dimension reduction by PCA on the highly variable genes. Data were further reduced to the 2D space on the first 20 PCs using uniform manifold approximation and projection (UMAP). Cell clusters were determined using a graph-based unbiased clustering approach implemented in Seurat. Positive markers defining each cluster were identified using Wilcoxon rank-sum test. Six representative markers were selected for each cluster to visualize in heatmaps.

### Single-Cell TCR Sequencing Data Analysis

After V(D)J sequences were assembled and annotated, only productive γ and δ TCR sequences were kept. Two TCR diversity metrics (i.e., species richness and exponential of Shannon entropy) were estimated for each sample using iNEXT R package (Hsieh et al. Methods in Ecology and Evolution, 2016. 7 (12): 1451-56; Chao et al., Ecological Monographs, 2014. 84 (1): 45-67). Species richness measured total unique clone numbers, whereas the Shannon index computed the uncertainty in predicting the identity of a sequence taken at random from the dataset. Both interpolated and extrapolated diversities were estimated, and 95% confidence interval was based on 50 bootstraps. TCR repertoires were visualized using the Treemap R package (https://CRAN.R-project.org/package=treemap). Downstream TCR analysis such as V(D)J usage, shared TCR, and integration of TCR and RNA-seq was performed using customized R scripts.

Bulk RNA-Sequencing and Data Analysis cDNA was synthesized and amplified (16-cycle PCR) from 5 ng total RNA using SMARTer® Ultra® Low RNA Kit (Clontech). Nextera XT library prep kit (Illumina) was used to generate the final sequencing library (12 PCR cycles performed to amplify libraries) using 1 ng of cDNA as the input. The amplified libraries were size-selected at 400˜600 bp. Sequencing was performed on Illumina HiSeq®2500 (Illumina) by multiplexed paired-read run with 2×100 cycles. The sequencing reads were mapped to the customized mouse genome using ArrayStudio (OmicSoft). Sense-strand exon reads were used to quantify the gene expression level by RSEM algorithm implemented in ArrayStudio. Genes were flagged as detectable with minimum of 10 reads. Differential expressed gene analysis was performed using Deseq2 (Love et al. Genome Biology, 2014. 15 (12): 550.). Genes with fold change |>1.5| and FDR <0.05 were considered significantly differentially expressed. The differentially expressed genes were subjected to pathway enrichment analysis using Running Fish exact test in NextBio (www.nextbio.com). TCR hypervariable-region sequences were reconstructed using TRUST (Li et al., Nature Genetics, 2017. 49 (4): 482-83.).

### Statistics

Statistical significance (p values) within the groups was determined by using one of the following statistical tests: unpaired t-tests assuming similar SD; one-way ANOVA with Tukey's multiple comparison post-test; or ordinary two-way ANOVA with Sidak's multiple comparison post-test, *p<0.05, ** p<0.005, *** p<0.0005. P values of <0.05 were considered significant. Statistical analyses were performed with Graphpad Prism 8. Samples were defined as biological replicates, and no technical replicates were used to generate graphs. Each experiment was repeated at least three times. Sample sizes and numbers and the statistical test used were indicated in each figure legend.

### Example 2

### Btnl2 is Preferentially Expressed in Small Intestinal Epithelial Cells

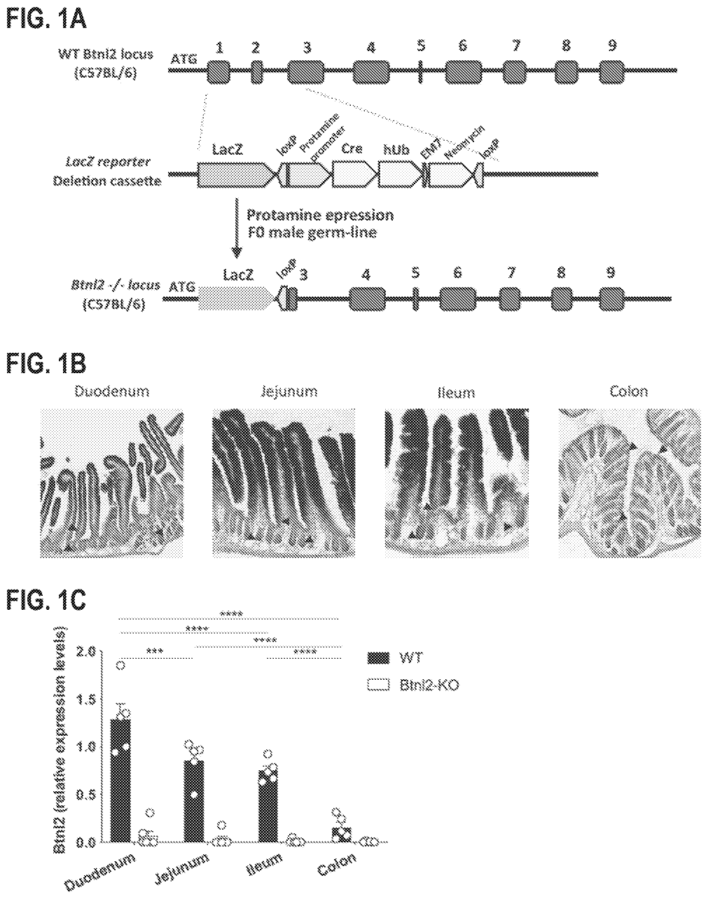

To determine the expression pattern of Btnl2 in the intestine during homeostasis, Btnl2-LacZ knock-in mice were generated and henceforth referred to as Btnl2-KO mice (FIG. 1A). As shown in FIG. 1B, Btnl2 was predominantly expressed in terminally differentiated IECs of the small intestine. Importantly, unlike Btnl1, Btnl4, and Btnl6 (Di Marco Barros et al., Cell, 2016. 167 (1): 203-218), Btnl2 expression was detected in duodenal Brunner's glands and duodenal, jejunal, ileal, and colonic crypts, indicating potential divergent roles of different Btnls dictated by their region-specific expression patterns (FIG. 1B). To further validate the observations, Btnl2 mRNA levels in IECs derived from terminally differentiated enterocytes isolated from duodenum, jejunum, ileum, and distal colon were measured. Btnl2 was highly enriched in duodenal IECs with a descending proximal-to-distal gradient, such that Btnl2 expression in IECs isolated from colon was ˜5-fold lower than in the ileum of WT mice (FIG. 1C). Similarly, BTNL2 transcripts were detected in small intestine, but not colon samples pooled from healthy human tissues.

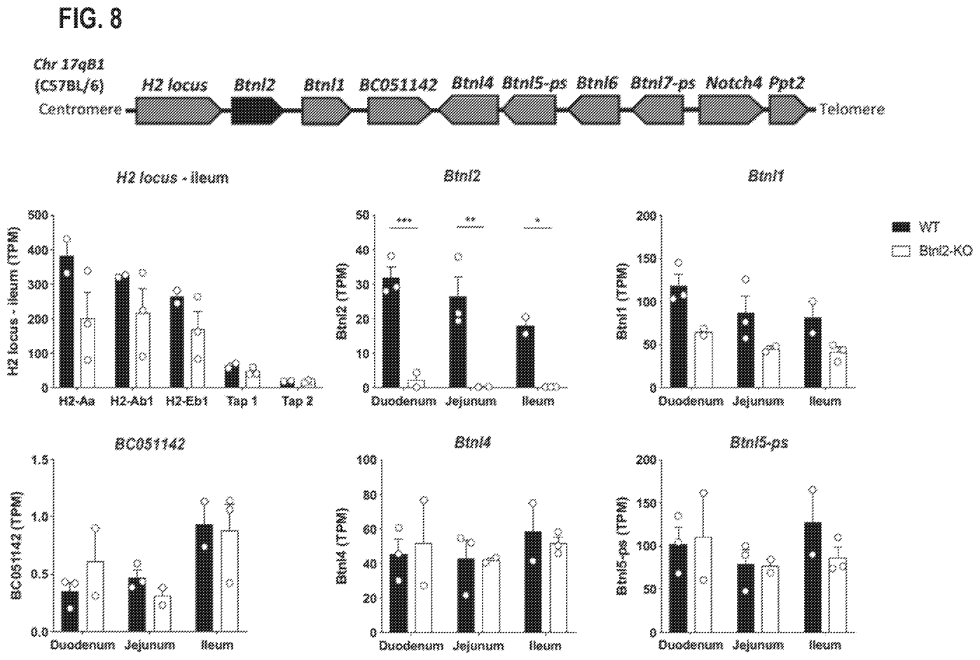

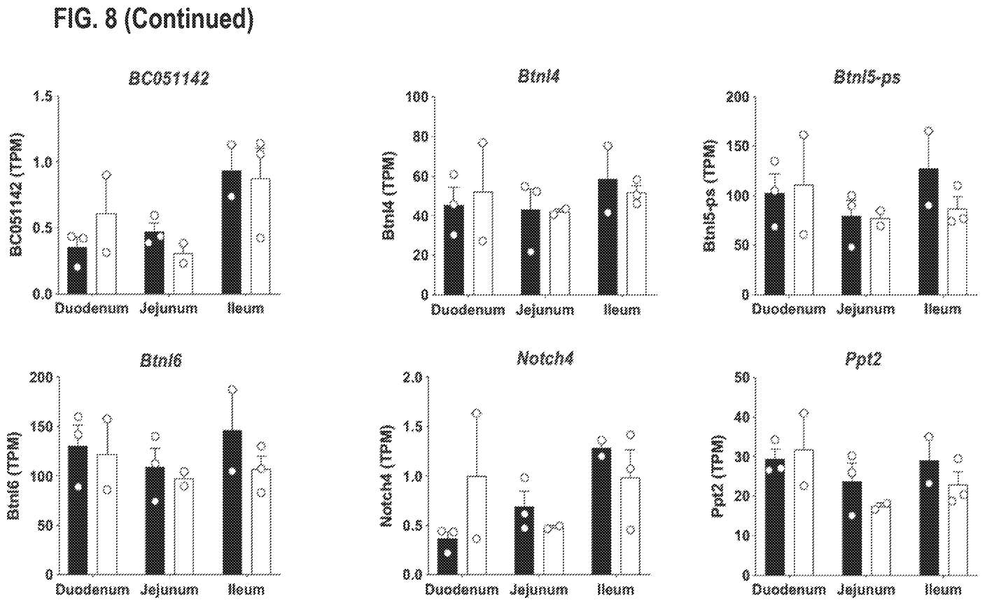

Given the close proximity of Btnl2 gene to H2 locus and other Btnls, the expression levels of several adjacent genes in the IEC fraction isolated from duodenum, jejunum, and ileum of Btnl2-KO and WT mice by bulk RNA-sequencing were investigated. No significant changes in H2-Aa, H2-Ab1, H2-Eb1, Tap1/2, BC051142, Btnl4, Btnl5, Btnl6, Notch4, and Ppt2 gene expression levels across different segments of the small intestine was observed, albeit Btnl2-KO mice displayed a trend towards decreased levels of Btnl1 (FIG. 8), indicating no significant coregulation of Btnl2 with adjacent genes near the H2 locus. Altogether, the data confirm preferential expression of Btnl2 in terminally differentiated enterocytes across different segments of the small intestine, indicating a compartment-specific function of Btnl2.

### Example 3

### Btnl2-KO Mice Display Increased Frequencies of γδ IELs Preferentially in the Ileum

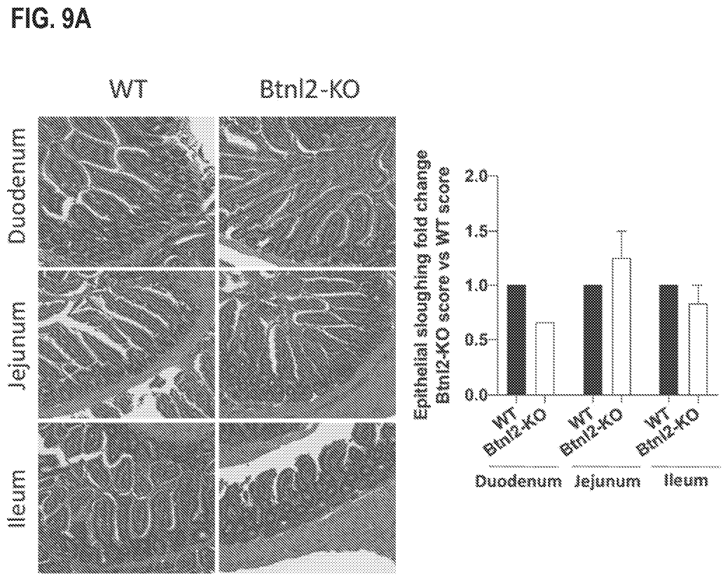

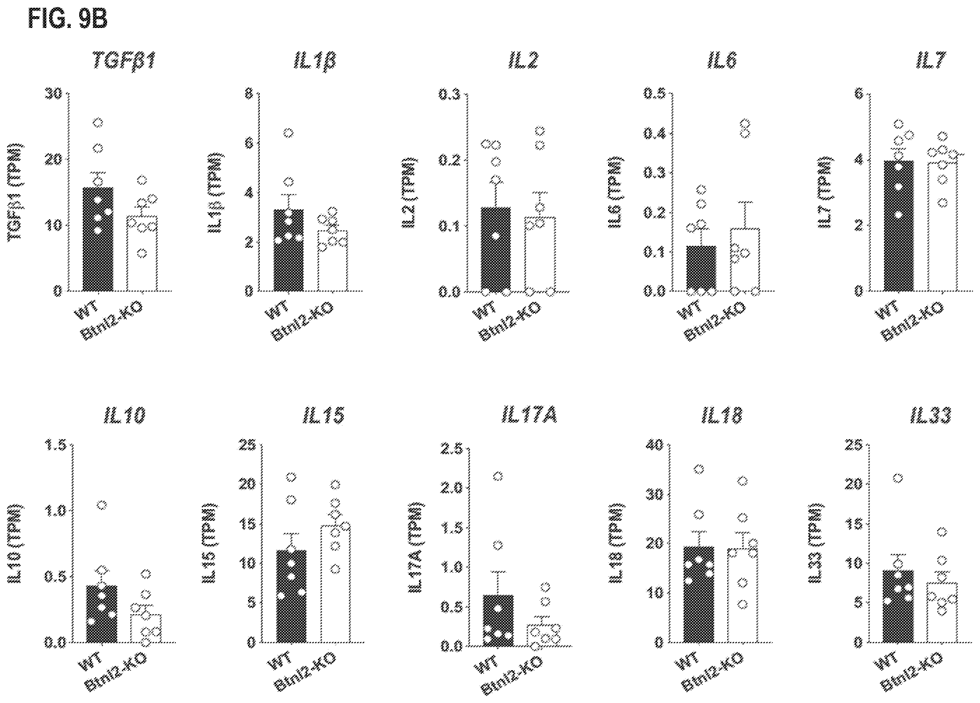

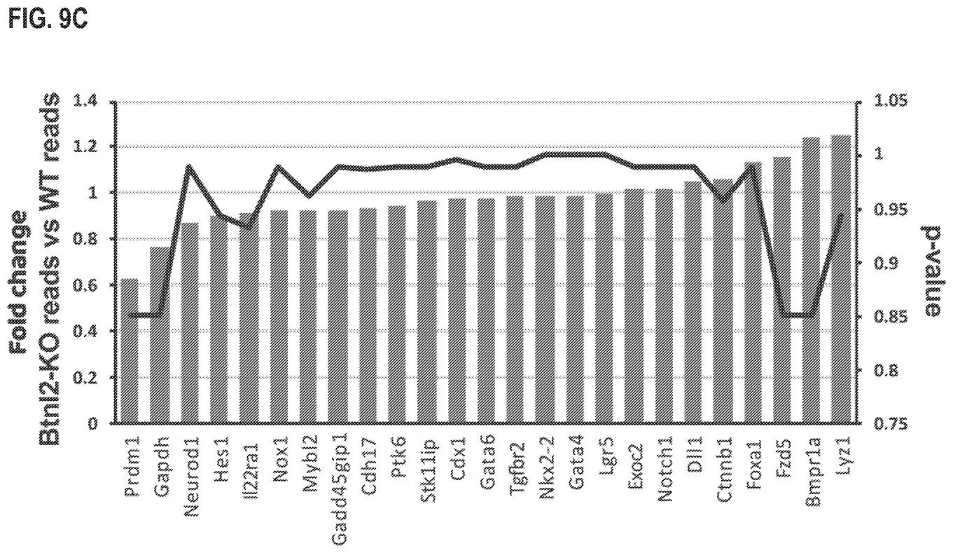

Under homeostatic conditions, Btnl2-KO mice did not exhibit any adverse intestinal pathology, as determined by body weight loss, increased epithelial sloughing, and pro-inflammatory cytokines (FIGS. 9A-B). In addition, no significant change in genes associated with differentiation and maturation of IECs was observed, indicating that IEC development and maintenance are not altered in unchallenged Btnl2-KO mice (FIG. 9C).

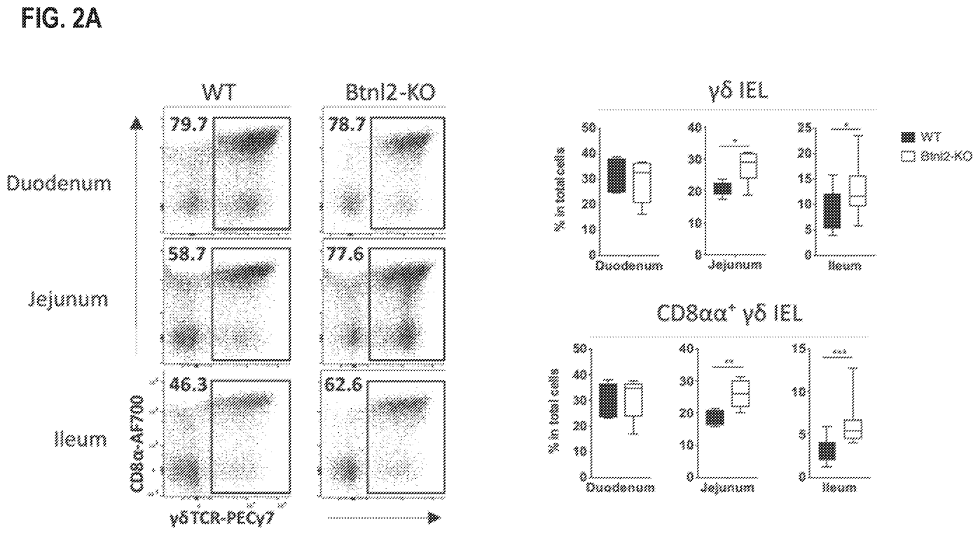

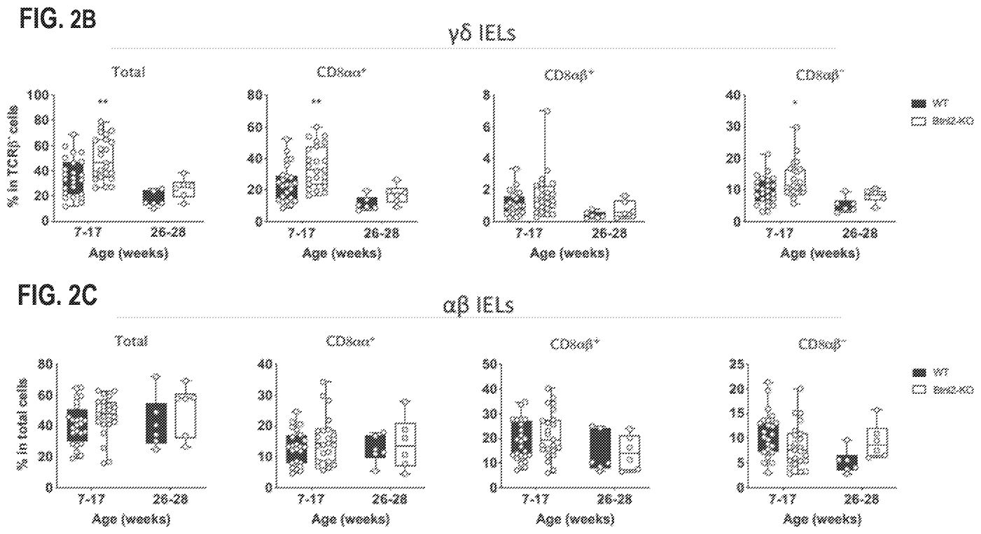

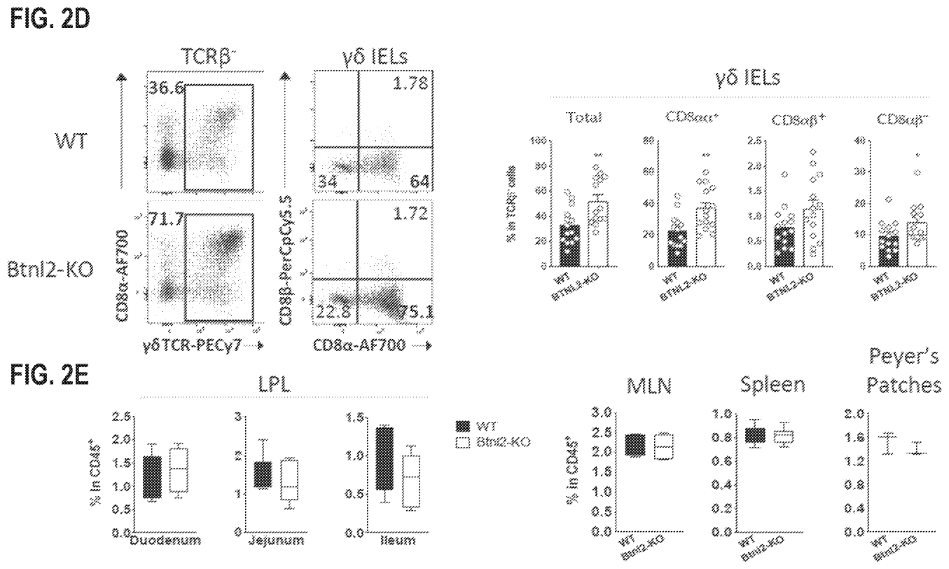

In light of the developing paradigm implicating members of Btnl/BTN/BTNL family in shaping the γδ T cell compartment and intrigued by the selective expression pattern of Btnl2 in different segments of small intestine, it was postulated that Btnl2 deficiency might impact the maintenance of γδ IEL subsets in different segments of the small intestine. To this end, IELs were isolated from duodenum, jejunum, and ileum of adult Btnl2-KO and WT mice. γδ IELs were found at ˜3-fold higher frequency in the duodenum compared to the ileum of WT mice (30.7% vs. 8.62% in total cells, FIG. 2A-right panel). However, it was observed that compared to WT littermates, Btnl2-KO mice displayed a 30-40% increase in the frequency of γδ IELs in the jejunum (27.1% vs. 20.1%) and ileum (12.8% vs. 8.6%), but not duodenum (29.4% vs. 30.7%) (FIG. 2A). Notably, this increase was observed predominantly in ileal CD8αα⁺ γδ IELs, indicating that Btnl2 may suppress their proliferative capacity in situ (FIG. 2A). As the number of Vγ7⁺ IELs have been reported to plateau in 11-16-wk-old young adults (Di Marco Barros et al., Cell, 2016. 167 (1): 203-218), whether the observed effect of Btnl2 on the percentage of ileal γδ IELs changed as mice approached middle adulthood was investigated. Previous work suggested that ileal γδ IEL frequencies, including CD8αα⁺ γδ IELs, remain steady past 6 months of age in WT mice. In contrast, it was observed that γδ IEL frequency was reduced by 50% at 6 months of age in WT mice in the facility (39.5% vs. 17.1% in total γδ IELs) (FIG. 2B). Conversely, αβ IEL frequency remained relatively unchanged at 6 months of age, indicating that αβ IELs actively maintain their levels, possibly through in situ expansion (FIG. 2C). It was found that γδ IELs were significantly increased in the ileum of young adult (up to 4 months old) Btnl2-KO compared to WT mice (36.9% vs. 22.3% in CD8αα⁺ γδ IELs), while mature adult mice displayed similar levels of γδ IELs (FIGS. 2B and 2D). αβ IELs were not significantly altered in Btnl2-KO mice during this time frame (FIG. 2C). As such, Btnl2 effect in young adult mice indicates it plays a role in γδ IEL maintenance under homeostatic conditions. Notably, γδ T cells were observed at comparable frequencies in the lamina propria (LP) of duodenum, jejunum, and ileum, mesenteric lymph nodes (mLN) and Peyer's Patches (PP) of Btnl2-KO and WT mice, indicating that Btnl2 exerts its function specifically on γδ CD8αα⁺ IELs (FIG. 2E).

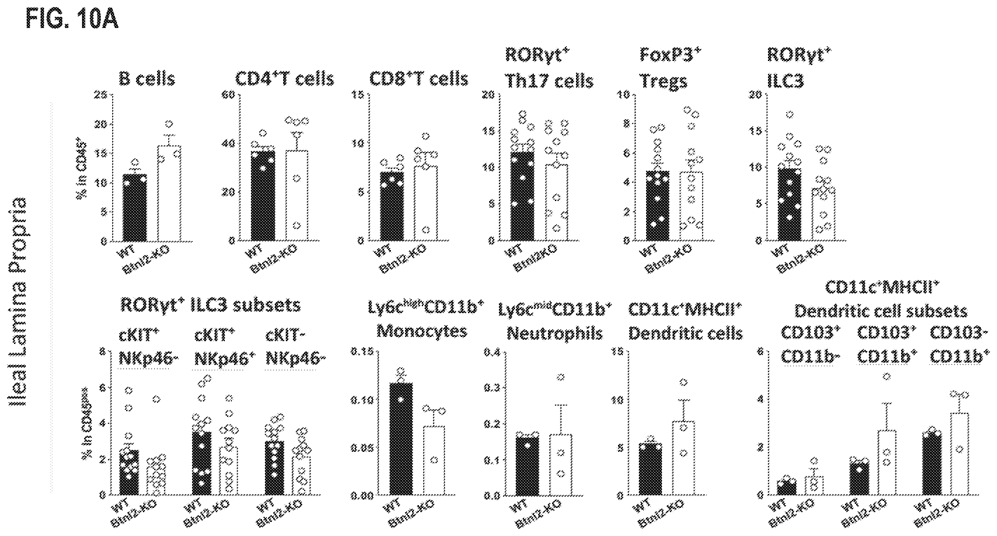

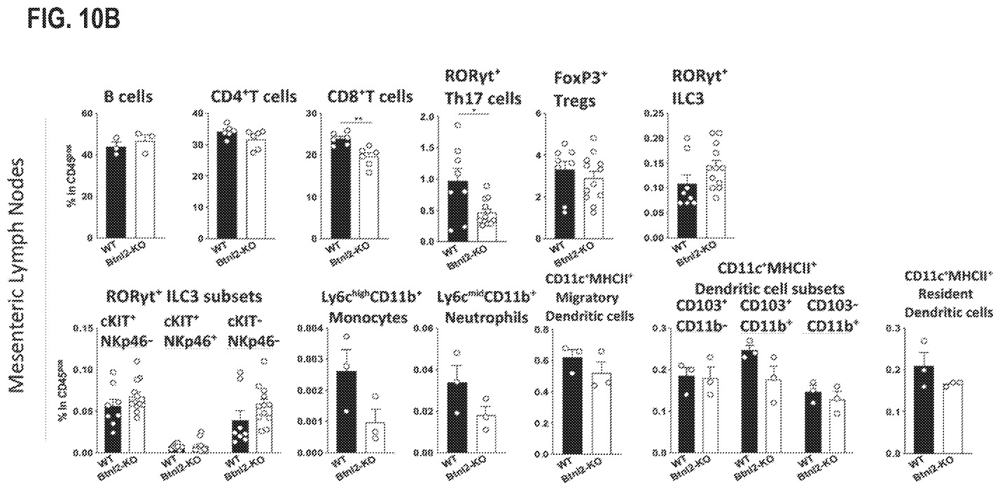

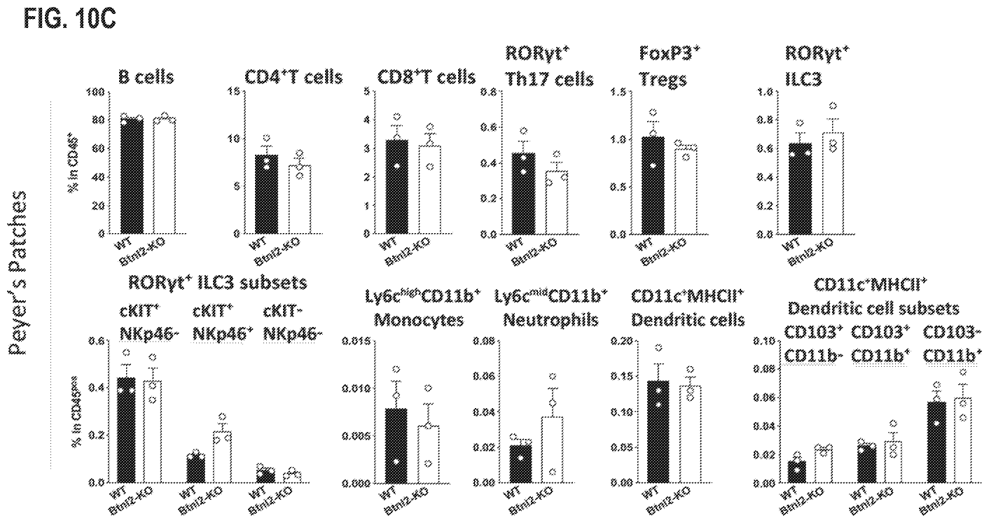

Prior studies had shown that recombinant Btnl2 can inhibit mLN CD4⁺T cell proliferation and promote Treg cell differentiation under certain activation conditions in vitro (Nguyen et al., J Immunol, 2006. 176 (12): 7354-60.). Nevertheless, similar frequencies of CD4⁺T cells and FoxP3⁺ Tregs in the ileal LP, mLNs, and PPs of Btnl2-KO mice were observed. In addition, Btnl2-KO mice exhibited comparable immune cell profiles across different tissues, emphasizing the specificity and localized effect of Btnl2 effects in the intestine on jejunal and ileal γδ IELS (FIGS. 10A-C).

### Example 4

### Btnl2 Suppresses Proliferation of Jejunal/Ileal γδ IELs

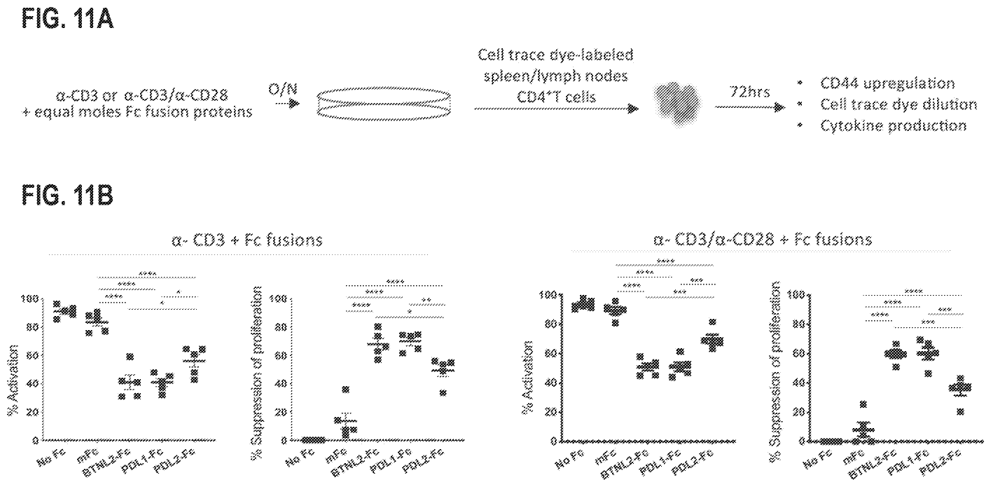

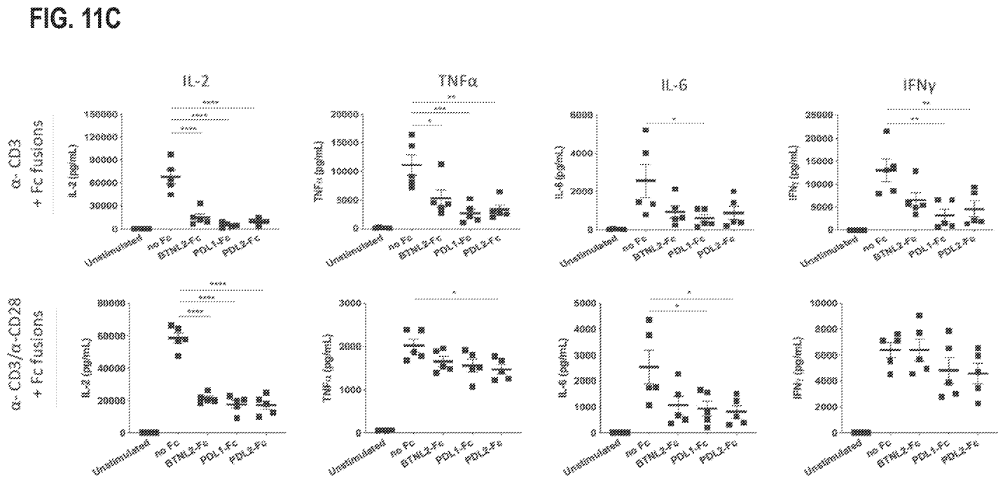

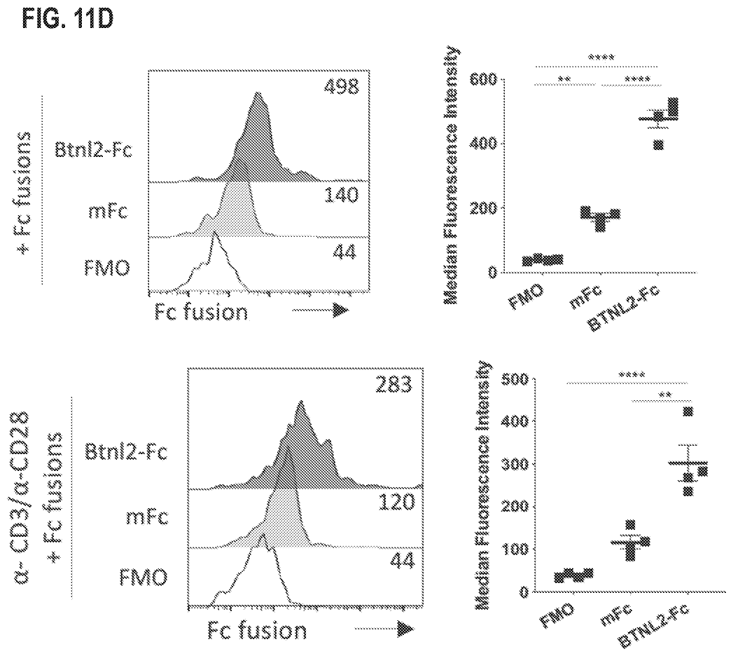

To investigate the effects that Btnl2 exerted on jejunal and ileal γδ IELs, its ability to suppress T cell proliferation was investigated. As Btnl2 inhibitory function is dependent on concurrent TCR stimulation and ligation with the putative Btnl2 receptor on CD4⁺ T cells in vitro, CFSE-labeled CD4⁺ T cells were activated in the presence of equimolar concentrations of plate-bound Btnl2-mFc, Pdl1-mFc, Pdl2-mFc or mFc (FIG. 11A). After 72 hours of culture, it was found that recombinant Btnl2 potently suppressed proliferation and activation of CD4⁺T cells similarly to Pull and Pdl2, as evidenced by CFSE dilution and greater than 40% decrease in cytokine production (FIGS. 11β-C). CD28 co-stimulation rescued production of TNFa and IFNg, but not IL-2 (FIG. 11C), which also coincided with ˜50% decrease in Btnl2 binding to its putative receptor on activated CD4⁺T cells (FIG. 11D).

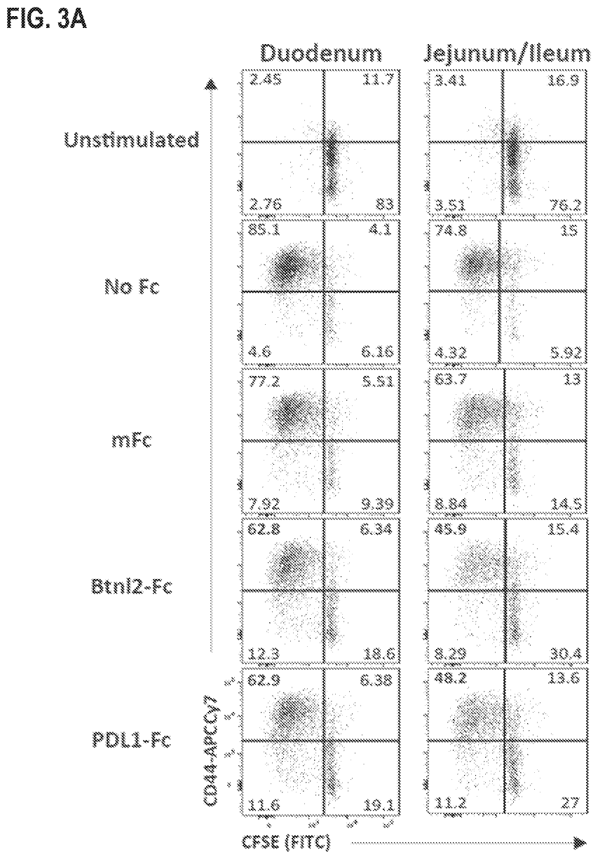

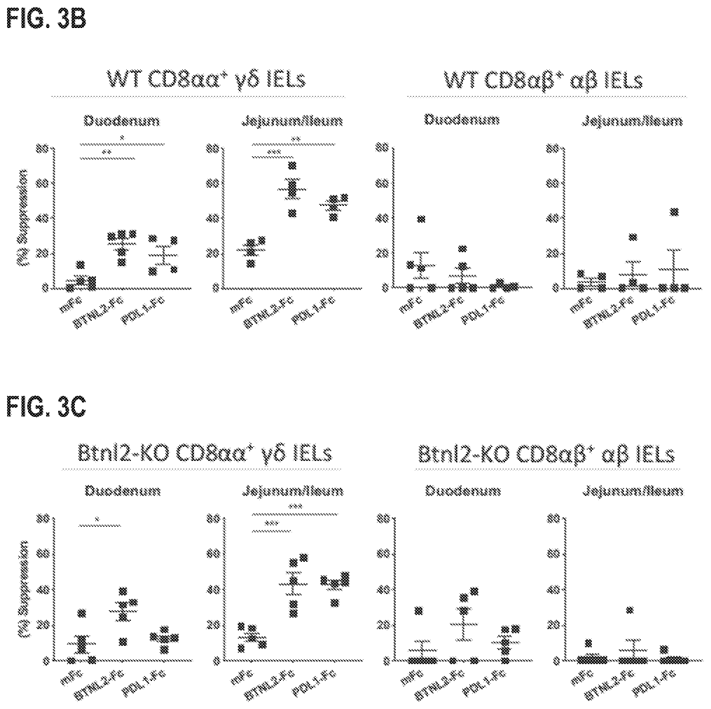

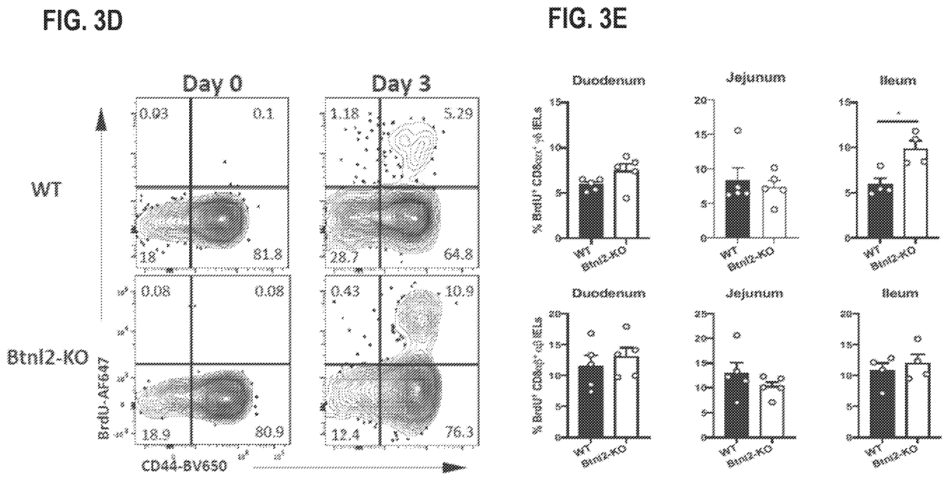

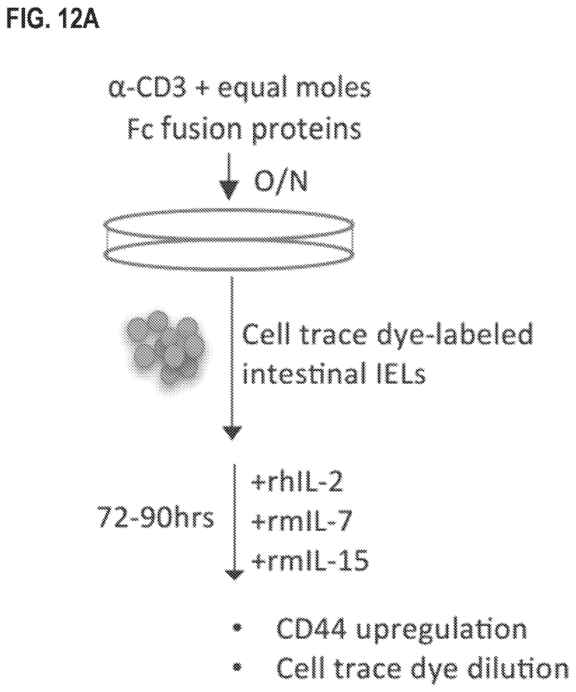

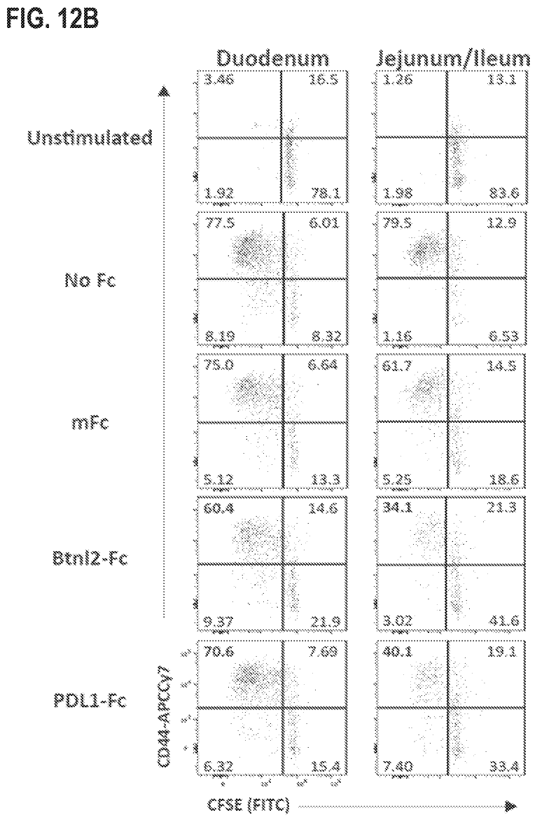

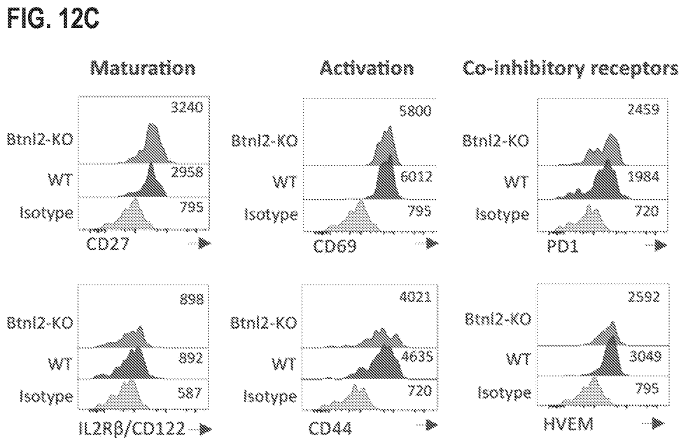

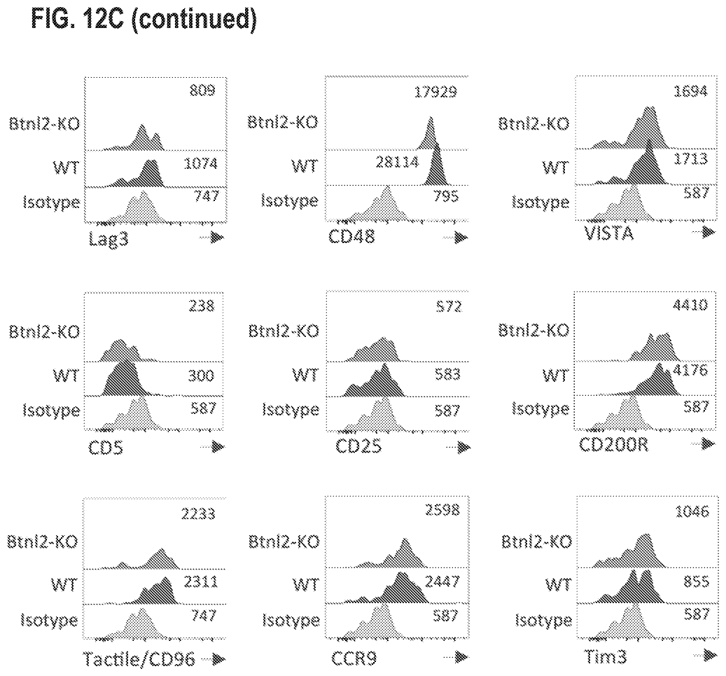

Next, whether Btnl2 suppresses the proliferation of γδ IELs in vitro (FIG. 12A) was determined. To obtain comparable numbers to those from duodenum, IELs were pooled from jejunum and ileum. As shown in FIG. 3A, duodenal CD8αα⁺ γδ IELs exhibited greater proliferative capacity compared to their jejunal/ileal counterparts indicating that duodenum and jejunal/ileal γδ IELs may require different TCR and/or cytokine stimulation. Recombinant Btnl2 and Pdl1 potently inhibited the proliferation of both duodenal and jejunal/ileal CD8αα⁺ γδ IELs (FIGS. 3A-B). However, Btnl2 suppressive effect was 2-fold higher on jejunal/ileal CD8αα⁺ γδ IEL proliferation compared to one observed for duodenal CD8αα⁺ γδ IELs (FIG. 3B). Importantly, recombinant Btnl2 failed to inhibit the proliferation of duodenal or jejunal/ileal CD8ab⁺ and CD8αα⁺ αβ IELs, indicating that the Btnl2 putative receptor may not be present on these cells (FIG. 3B). Contrary to previous reports indicating stronger responsiveness of Btnl1-KO γδ IELs to α-CD3 stimulation (Di Marco Barros et al., Cell, 2016. 167 (1): 203-218), it was found that duodenal and jejunal/ileal Btnl2-KO γδ and αβ IELs exhibited equal proliferative capacity compared to their WT counterparts, indicating that Btnl2 deficiency did not impair the ability of IELs to respond to TCR and cytokine stimulation (FIG. 12B). Moreover, recombinant Btnl2 similarly inhibited the proliferation of Btnl2-KO and WT jejunal/ileal γδ IELs in vitro (FIG. 3C). Btnl2-KO and WT jejunal/ileal γδ IELs also showed comparable expression profiles of coinhibitory receptors (e.g., PD1) and markers associated with tissue residence, maturation, and activation (e.g., CD69, CD44, CD27, CD122) (FIG. 12C). Altogether, these data indicate that Btnl2 preferentially suppresses jejunal/ileal CD8αα⁺ γδ IEL proliferation.

To determine the proliferative capacity of Btnl2-KO γδ IELs in vivo, BrdU incorporation in γδ IELs was measured. As shown in FIGS. 3D-E, high BrdU incorporation was observed in WT CD8αα⁺ γδ IELs by day 3. Notably, ileal Btnl2-KO γδ IELs incorporated BrdU ˜2-fold more than their WT counterparts (9.86±1.52 vs. 5.87±1.18), emphasizing the enhanced proliferative capacity of γδ IELs in the absence of Btnl2 (FIG. 3D, E). Overall, these observations indicate that Btnl2 may serve as a γδ immunocheckpoint molecule by limiting the expansion of mature γδ IELs in the ileum and, potentially and regulate their effector responses during the normal epithelial lifespan.

### Example 5

### Ileal Btnl2-KO γδ IELs Display a Subdued Antibacterial Response Module

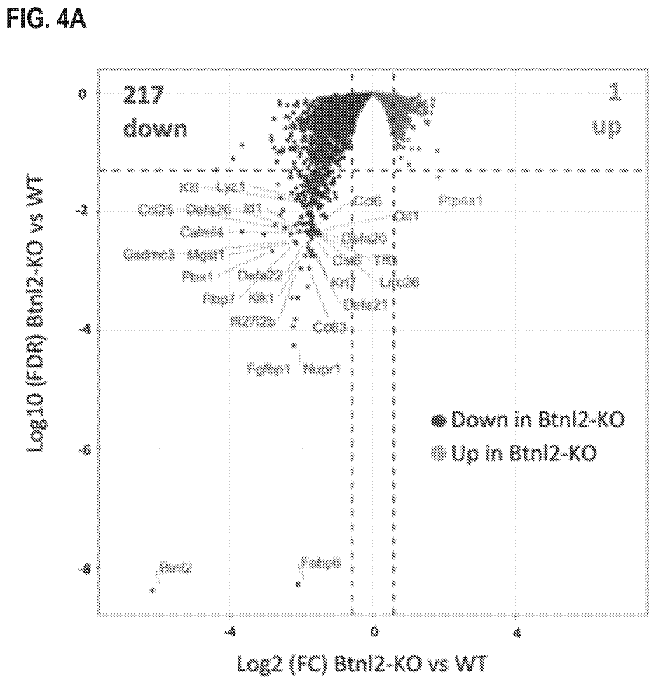

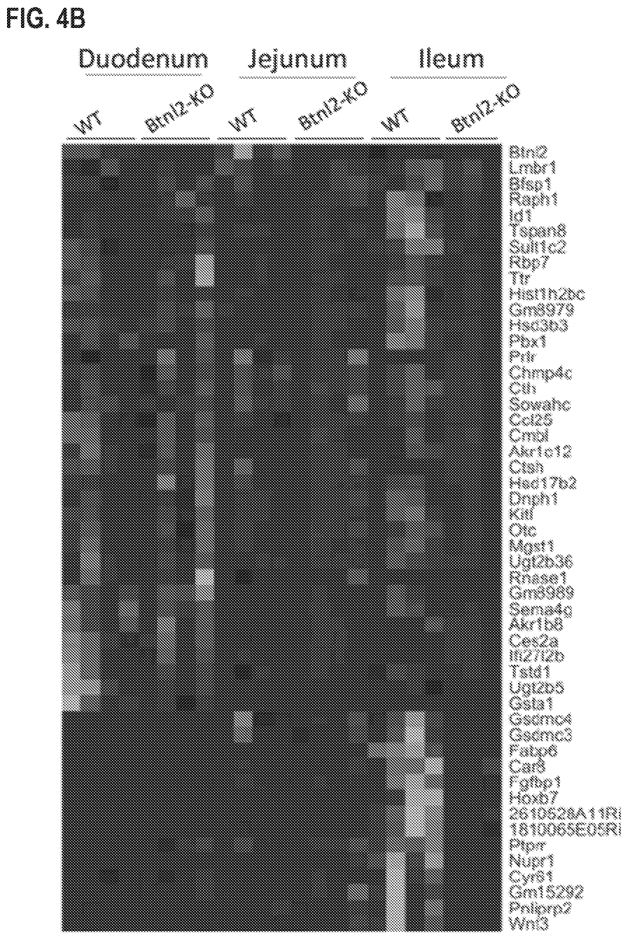

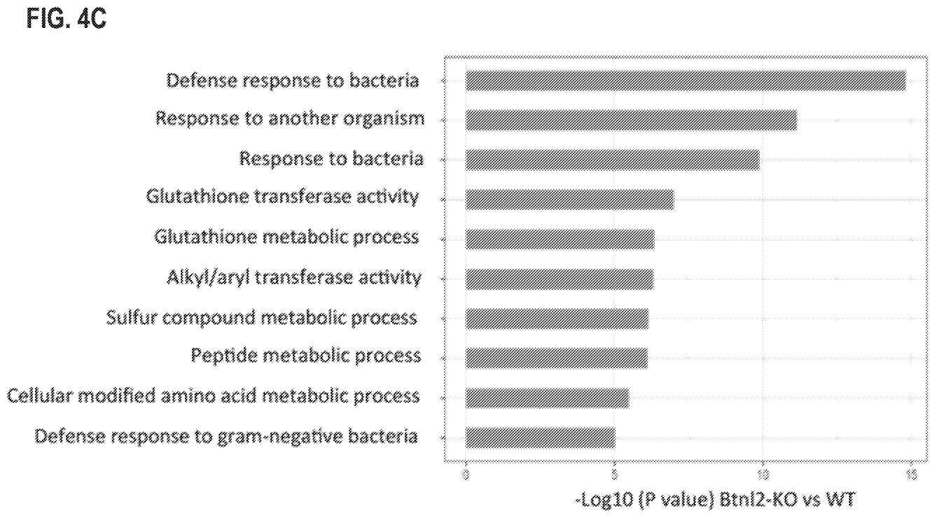

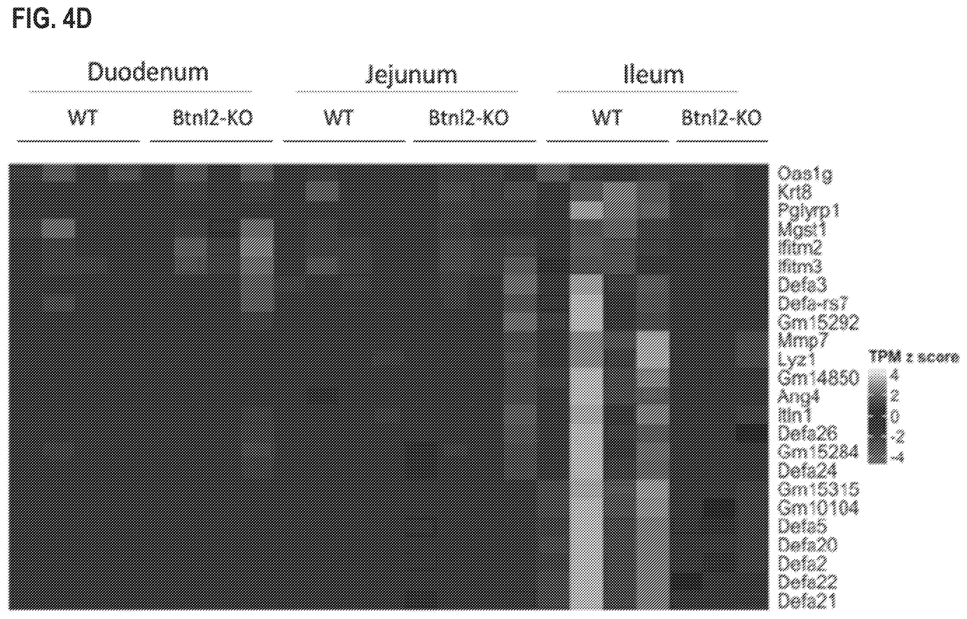

To gain insights into the impact of Btnl2 deficiency on γδ IEL cytolytic potential, transcriptomic analysis of γδ IELs enriched from duodenum, jejunum, and ileum of 11-wk-old cohoused Btnl2-KO and WT mice at steady-state were performed. Over 200 genes were significantly downregulated (FDR<0.05 and fold change >1.5) in ileal Btnl2-KO γδ IELs compared to their WT counterparts (FIG. 4A), whereas only Btnl2 was significantly decreased in duodenal and jejunal Btnl2-KO γδ IELs (FIG. 4B). The top 50 downregulated genes included signaling molecules (e.g., Raph1, Cyr61, Tspan8), transcriptional regulators of cell proliferation and apoptosis (e.g., Id1, Pbx1, Nupr1, Hoxb7), growth factors (e.g., Kit1, Wnt3, Fgfbp1), antimicrobial molecules (e.g., Ifi2712b, Ccl25, Gsdmc3/4) and different classes of metabolic molecules (e.g., aminoacid-Mgst1, lipid-Fabp6, Pnliprp2, sulfur-Sult1c2, Cth, carbonic anhydrases-Car8) (FIG. 4A-B). Collectively, these observations hint at some decrease in metabolic function of ileal Btnl2-KO γδ IELs compared to ileal WT γδ IELs.

In line with these findings, gene ontology enrichment analysis revealed that the most significantly dysregulated biological processes centered around bacterial tolerance and clearance, emphasizing that ileal Btnl2-KO γδ IELs display an impaired ability to secrete antimicrobial molecules at a steady state (FIG. 4C). Interferon-induced molecules and several members of α-defensin antimicrobial peptide family were found among the genes significantly downregulated in ileal Btnl2-KO γδ IELs compared to their WT counterparts (FIG. 4D). Hence, the findings indicate that γδ IELs in the ileum, but not duodenum or jejunum, may be specialized in secreting antibacterial molecules in response to local microbial antigens.

### Example 6

### Single-Cell TCR Sequencing Highlights Greater Repertoire Diversity in Btnl2-KO γδ IELs

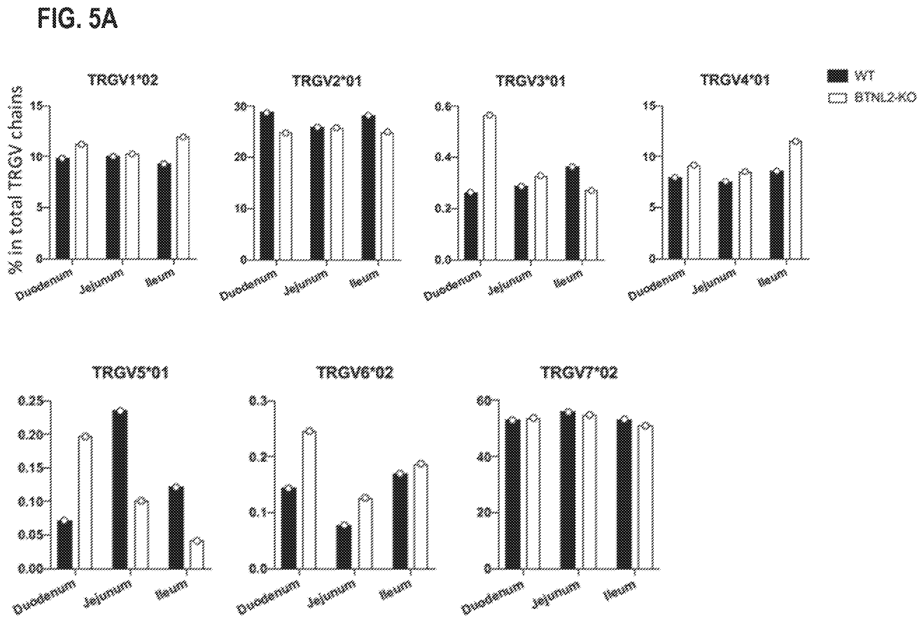

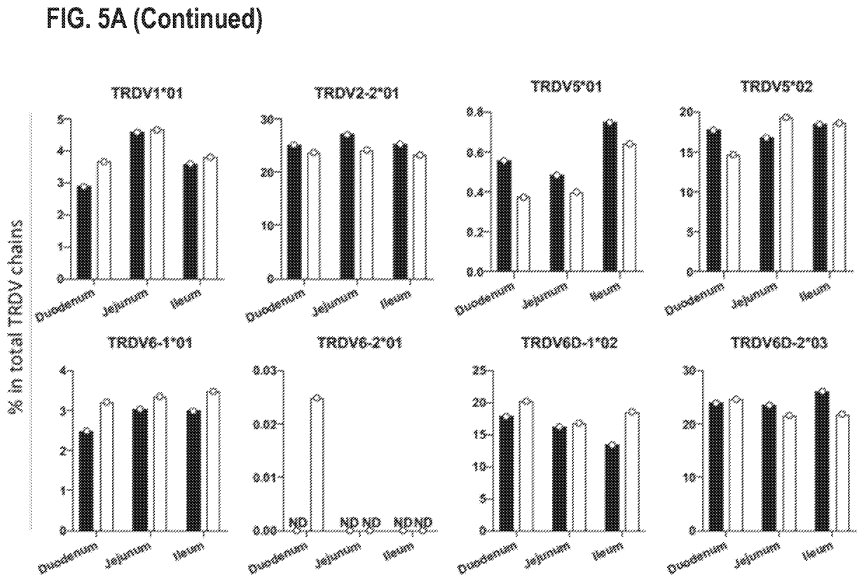

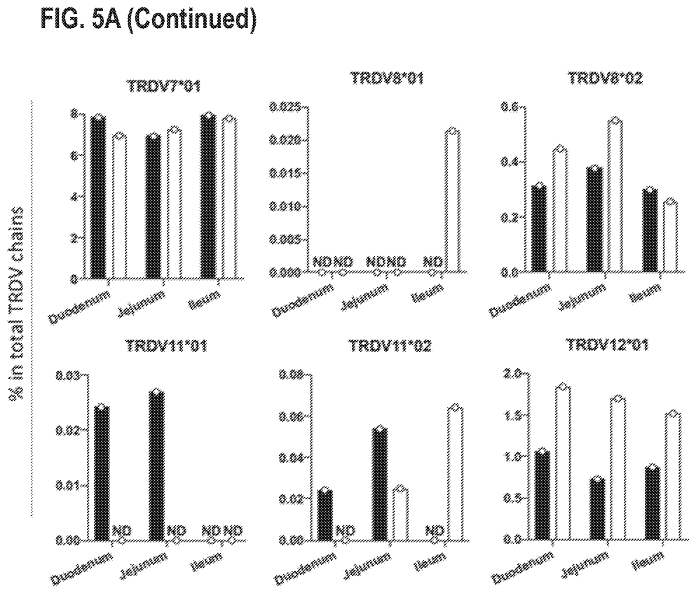

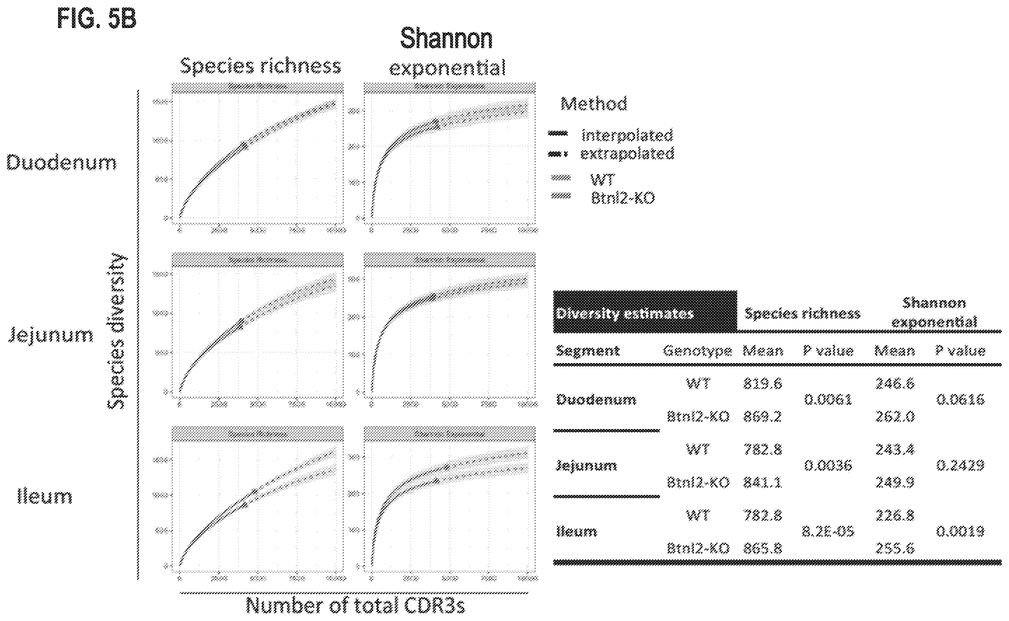

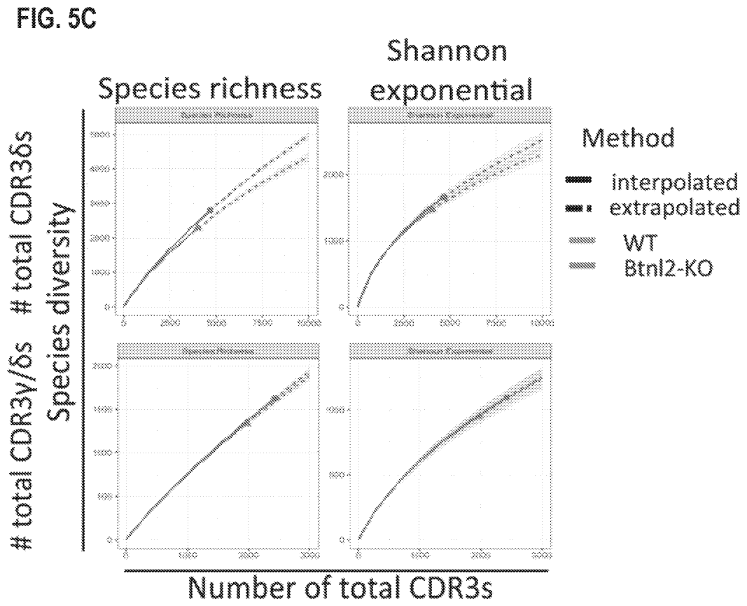

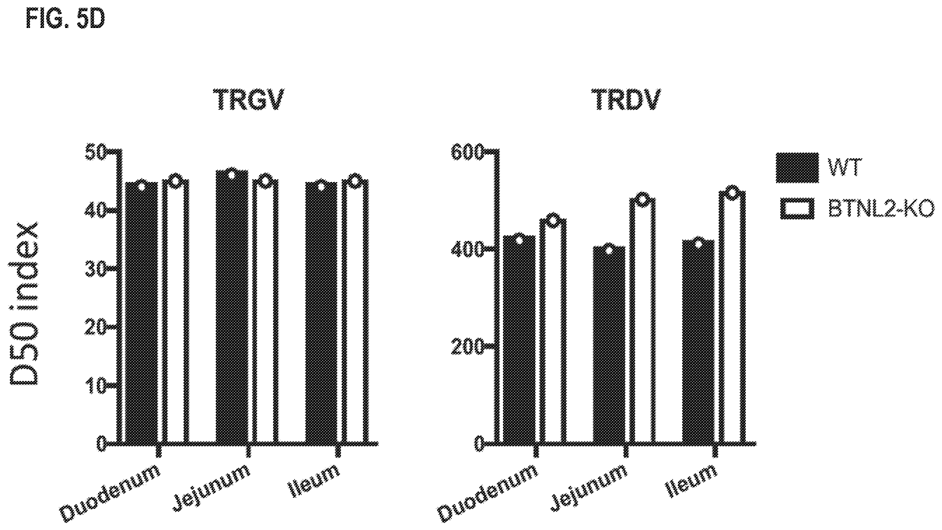

To determine whether the downregulated antibacterial response module observed in ileal Btnl2-KO γδ IELs related to an altered γ/8 TCR repertoire, unbiased single-cell TCR sequencing on duodenal, jejunal, and ileal γδ IELs from Btnl2-KO and WT mice was performed. In total, 28,679 cells were sequenced, and 24,961 productive γ chains and 24,515 productive δ chains were reassembled. Among all sequenced cells, 17,260 cells (60.2%) had paired γ and δ chains. It was found that TRGV and TRDV gene usage was comparable between Btnl2-KO and WT γδ IELs in the duodenum, jejunum, and ileum (FIG. 5A). TRGI7 gene usage averaged 50% of TCR γ chains, whereas TRDV 2-2, TRDV5, TRDV6D-1, and TRDV6D-2 genes were equally represented, and their combined gene usage surpassed 80% of TCR & chains. Ileal Btnl2-KO γδ IELs used TRGV7 gene less frequently than their WT counterparts (51.0% vs. 53.2%). However, they employed the less common TRGV4 gene more frequently (11.5% vs. 8.6%; Pearson's chi-squared test, p=1.78×10⁻⁷). Similarly, ileal Btnl2-KO γδ IELs showed reduced usage of TRDV2-2 and TRDV6D-2 genes compared to their WT counterparts (23.2% vs. 25.3%; 21.9% vs. 26.2%, respectively), whereas usage of TRDV6D-1 gene (18.6% vs. 13.4%) and of the less employed TRDV12 gene (1.52% vs. 0.87%) increased.

To establish TCR γ chain and δ chain clonotypes, TCR γ and δ sequences encoded by the same V gene and J gene segments with identical aminoacid sequences in the third complementarity determining regions (CDR3) were identified. Using the R iNext package (Hsieh et al. Methods in Ecology and Evolution, 2016. 7 (12): 1451-56), two metrics were computed (Species richness and Shannon diversity) to estimate TCR diversity for each sample. Overall, ileal Btnl2-KO γδ IELs had consistently higher TCR γ chain diversity than WT γδ IELs by both measurements in both interpolated and extrapolated data. For example, 3500 γ chains from each sample by 50 bootstrap replications were randomly sampled, and it was observed that the mean Shannon diversity of ileal Btnl2-KO γ chains was 255.6, significantly higher than 226.8 in WT (p=0.0019, T test) (FIG. 5B). Duodenal and jejunal Btnl2-KO γδ IELs also showed a trend towards higher diversity in TCR γ chain compared to WT γδ IELs, but the difference was marginal when contrasted to their ileal counterparts (p=0.0616 and 0.2429, respectively for Shannon diversity when sampling 3500 γ chains, FIG. 5B). Although ileal Btnl2-KO γδ IELs had increased diversity in TCR δ chains (FIG. 5C), Btnl2-KO γδ IELs isolated from all three segments displayed an increased frequency of unique clonotypes that comprised 50% of the TRDV repertoire (duodenum: 19.0% vs. 17.6%; jejunum: 20.2% vs. 18.6%; and ileum: 18.5% vs. 18%) (FIG. 5D). In contrast, the overall repertoire diversity of paired γ/8 chains was marginally altered in ileal Btnl2-KO γδ IELs (FIG. 5C). Collectively, these results indicated that the ileal TCR γδ repertoire diversity may be continually shaped by both host and microbial antigens and metabolites such that fluctuations in the frequencies of ileal γδ IELs, as well as perturbations in the antimicrobial response module, could lead to significant clonal revisions.

### Example 7

### Shared TCR Clonotypes Display Different Frequencies in Ileal Btnl2-KO and WT γδ IELs

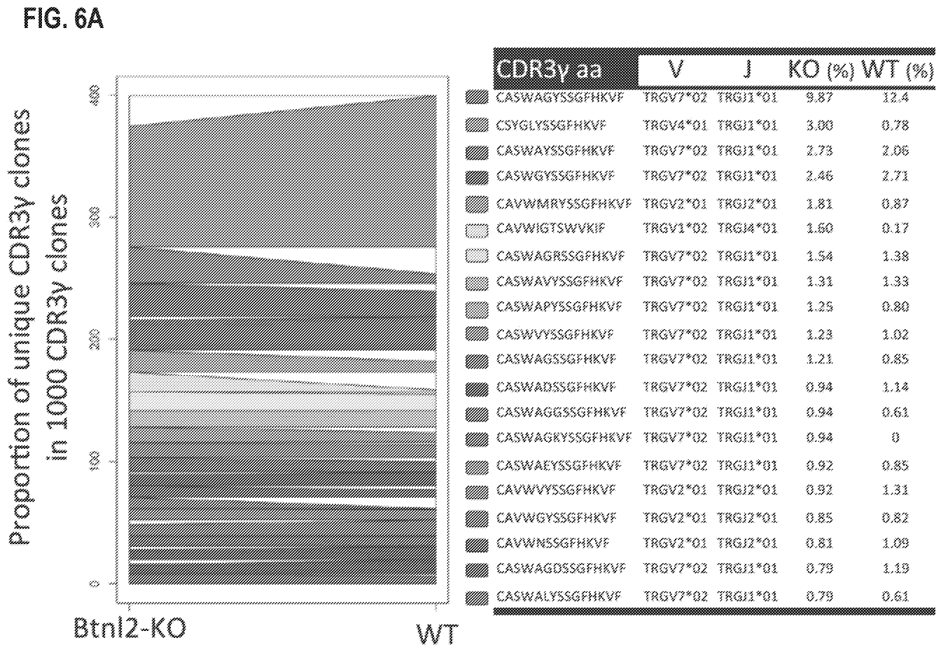

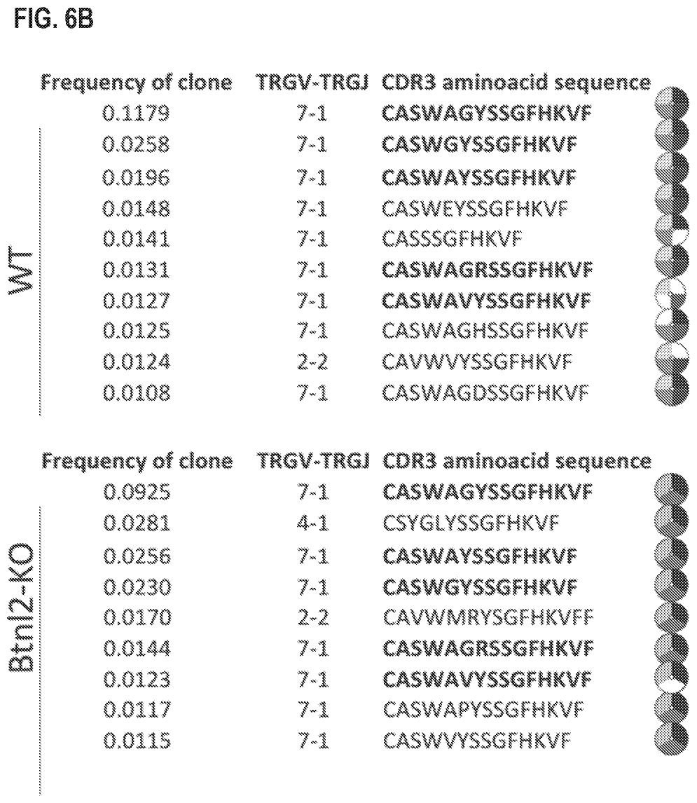

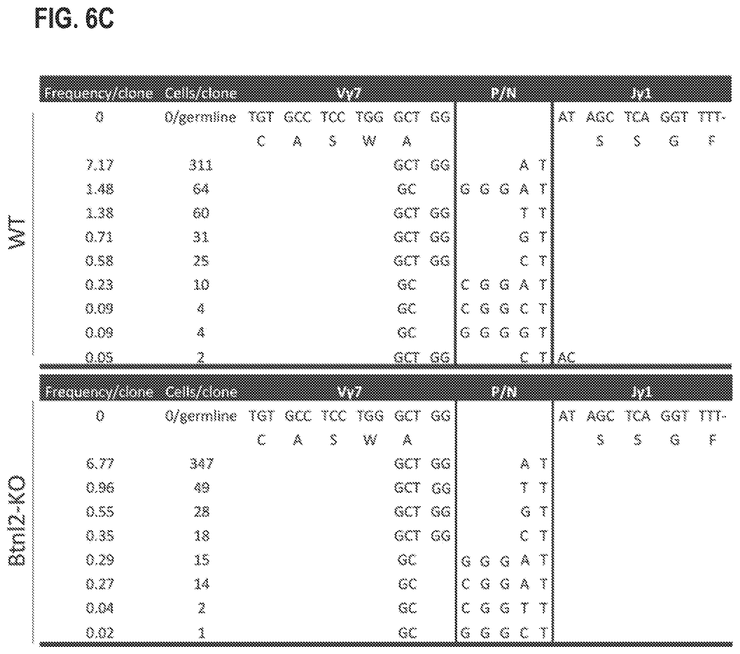

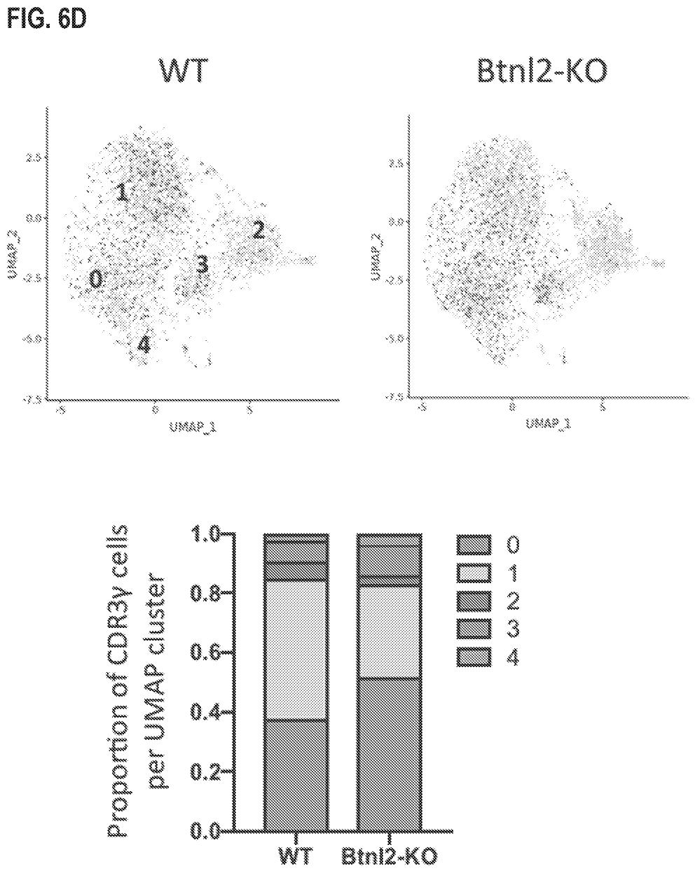

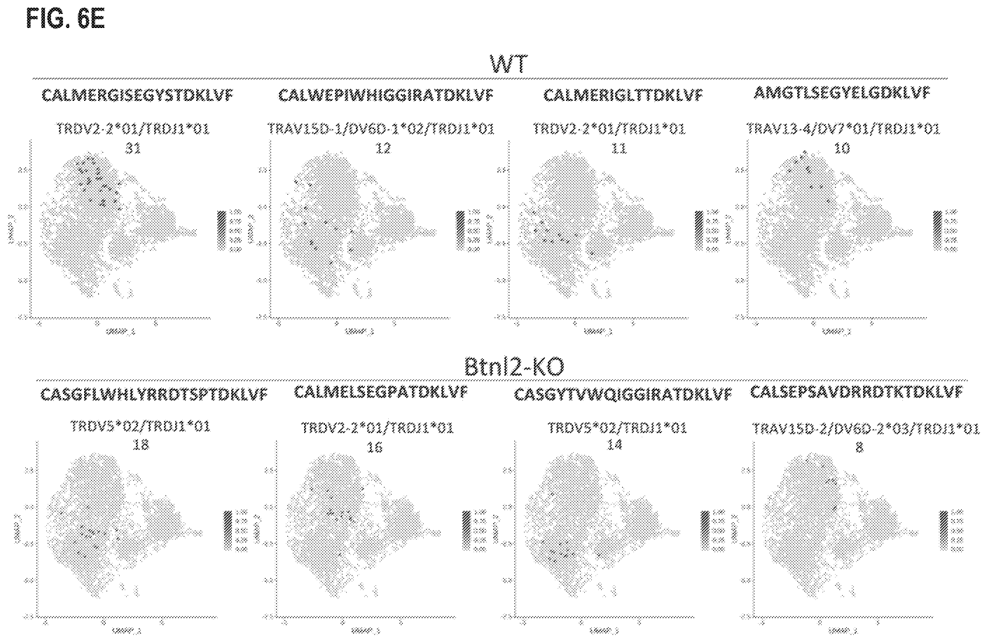

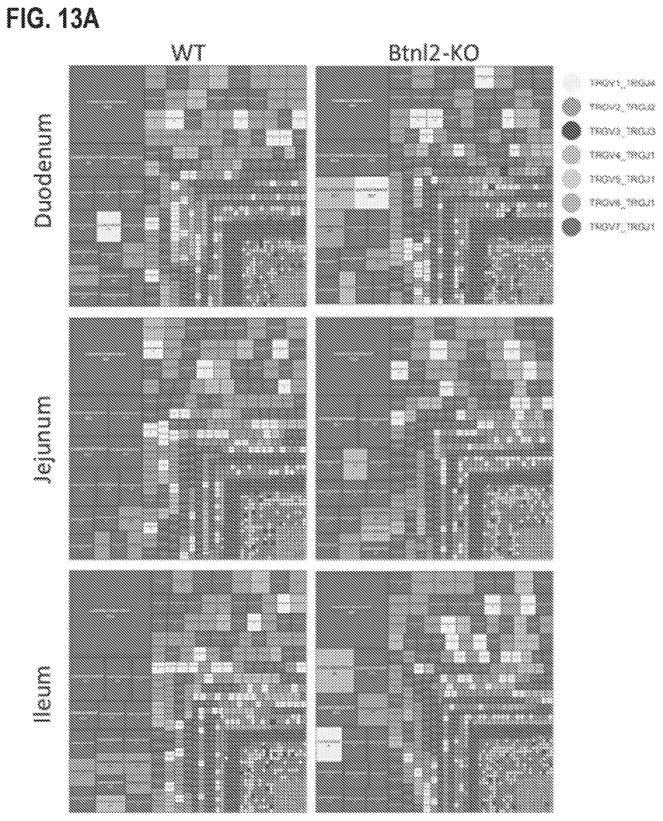

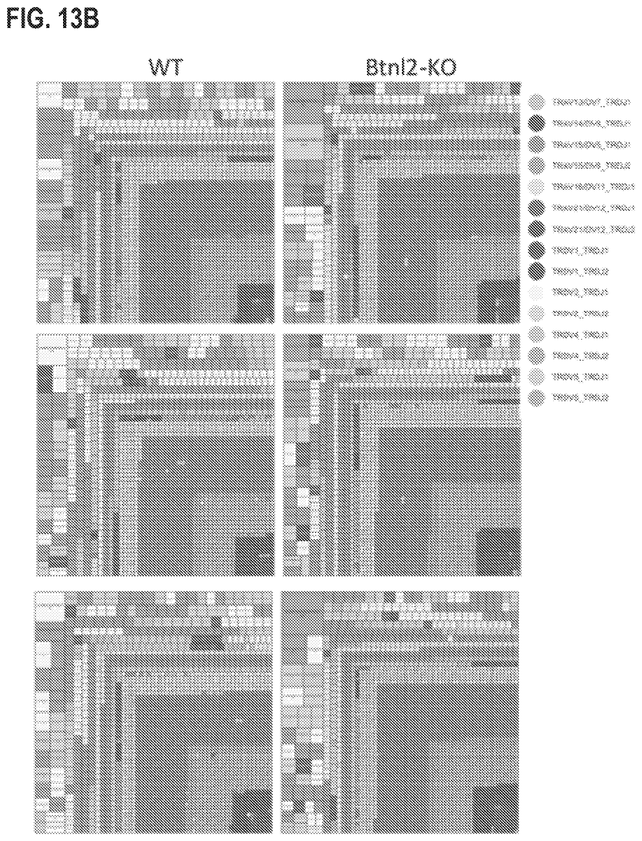

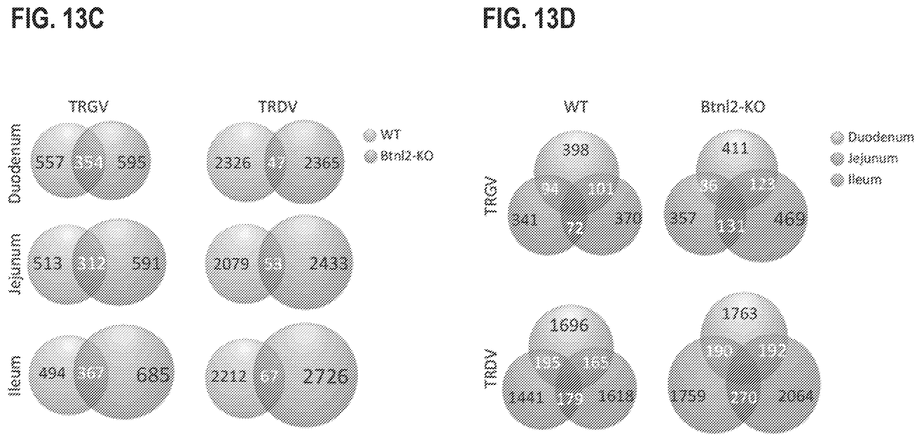

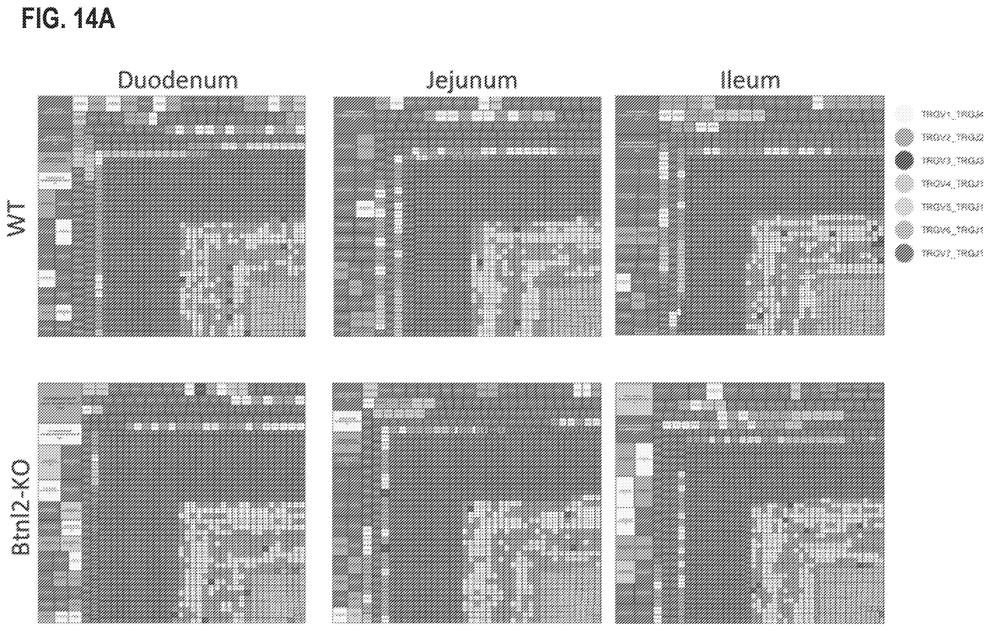

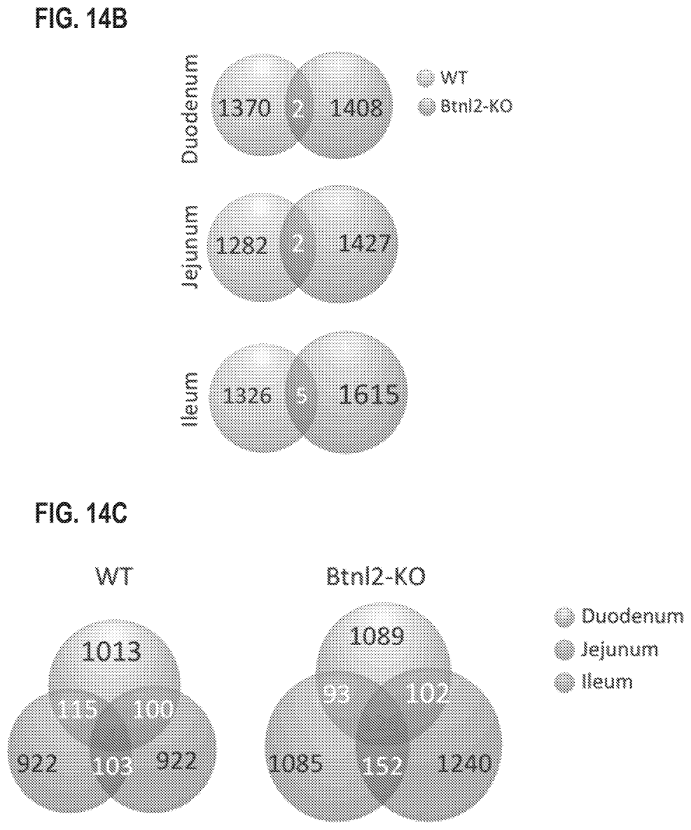

In addition to unique Btnl2-KO CDR3γ clones, 19 of the top 20 ileal Btnl2-KO CDR3γ clones were shared by ileal WT γδ IELs as contracted or expanded clones, possibly contributing to the TRGV repertoire diversity in Bin/2-KO compared to WT γδ IELs (FIG. 6A). Overall, ˜40% of CDR3γ clones were shared by Bin/2-KO and WT γδ IELs in each segment (FIGS. 13A-C). In TCRδ chains, each segment was characterized by a large number of unique CDR3δ clones and fewer than 3% shared clones between Bin/2-KO and WT mice (FIGS. 13B-C). More CDR3γ and CDR3δ clones were shared by jejunal and ileal γδ IELs (131 vs. 72 and 270 vs. 179, respectively) in Bin/2-KO compared to WT mice, highlighting the jejunum as a transitional segment in the small intestine (FIG. 13D). Bin/2-KO and WT γδ IELs carrying one CDR3γ/δ pair showed virtually no overlap (less than 0.4%) of their CDR3γ/δ clonal repertoire, emphasizing that individual mice carry unique γ-chain-δ-chain pairings (FIGS. 14A-B). Nevertheless, up to 13% of CDR3γ/δ paired clones overlapped between two or all three segments, indicating the presence of dominant CDR3γ/δ pairs that populate all segments within individual mice (FIG. 13C).

Next, CDR3γ sequences were reconstructed using bulk RNA-seq data from each individual mouse. The most frequent ileal CDR3γ clones revealed by single cell TCR sequencing data were also found in different individual mice, which indicated that the single cell TCR repertoire was an accurate representation of individual Btnl2-KO and WT CDR3γ diversities (FIG. 6B). The top ileal Btnl2-KO CDR3γ clones also included TRGV4, TRGV1, and TRGV7 genes, whereas ileal WT CDR3γ clones carried TRGV7 almost exclusively (FIG. 6C). For the most frequent CDR3γ chain (Vγ7-J1, CASWAGYSSGFHKVF (SEQ ID NO: 29)), ˜70% of IELs carrying this CDR3γ amino acid sequences were translated from the same DNA sequence, which could result from clonal expansion of one progenitor or from recurrent independent recombinations that pair with distinct V8 sequences in each clone, while the remainder of the IELs derived from smaller clones with different DNA sequences (FIG. 6D). Similar convergent Vγ recombination has been observed for common human Vγ9⁺ clonotypes, where their abundance has been proposed to be preconfigured since birth. Likewise, the most prevalent Vγ7⁺ clonotype stemmed from one major and several minor independent convergent recombination events. These findings highlighted the presence of public TRGV clonotypes of γδ IELs and indicated that the paired TRGV/TRDV repertoire diversity may be driven by the CDR3δ sequence.

### Example 8

γδ IEL Transcriptome of Shared CDR3γ is Shaped by Pairing with CDR3δ

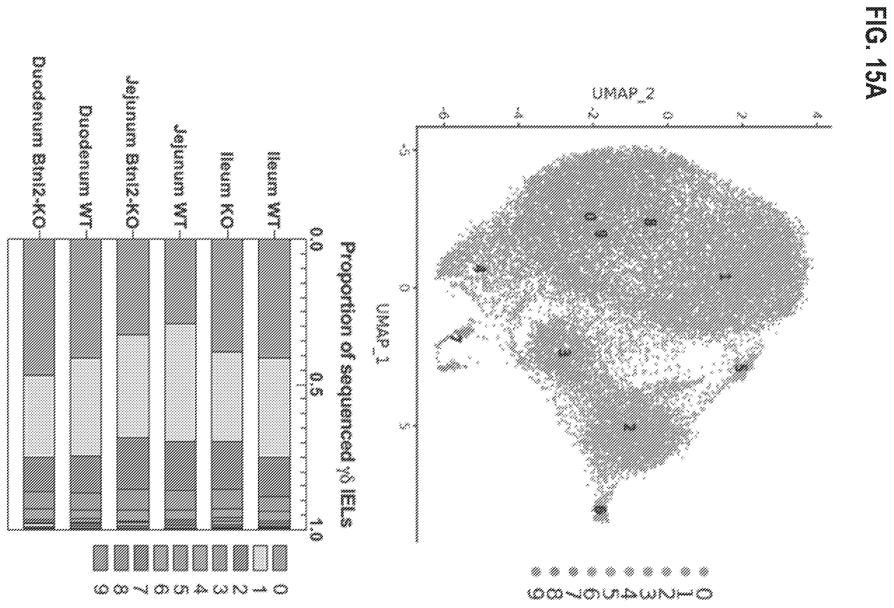

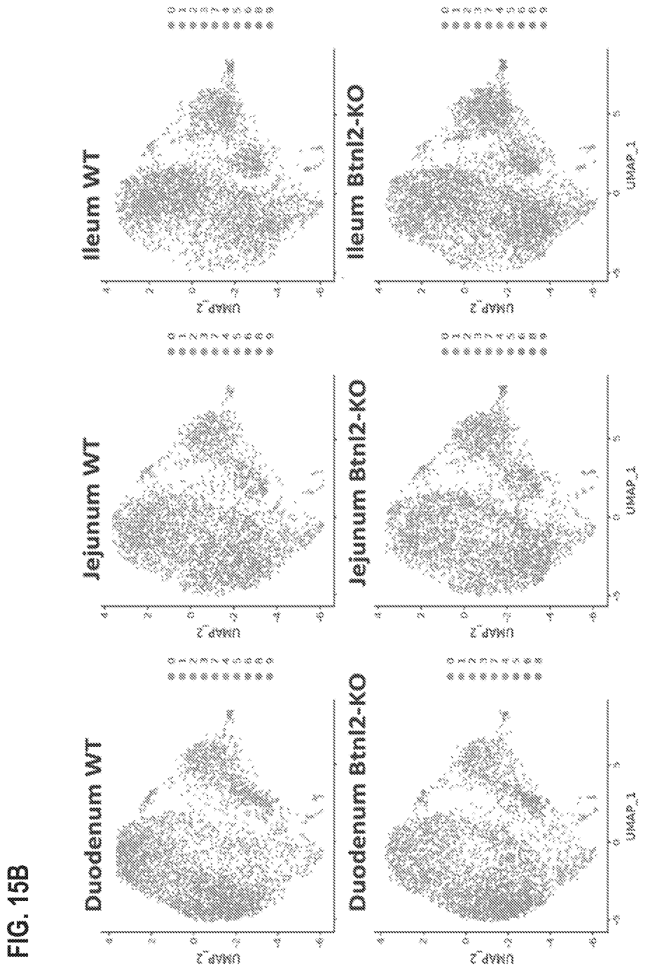

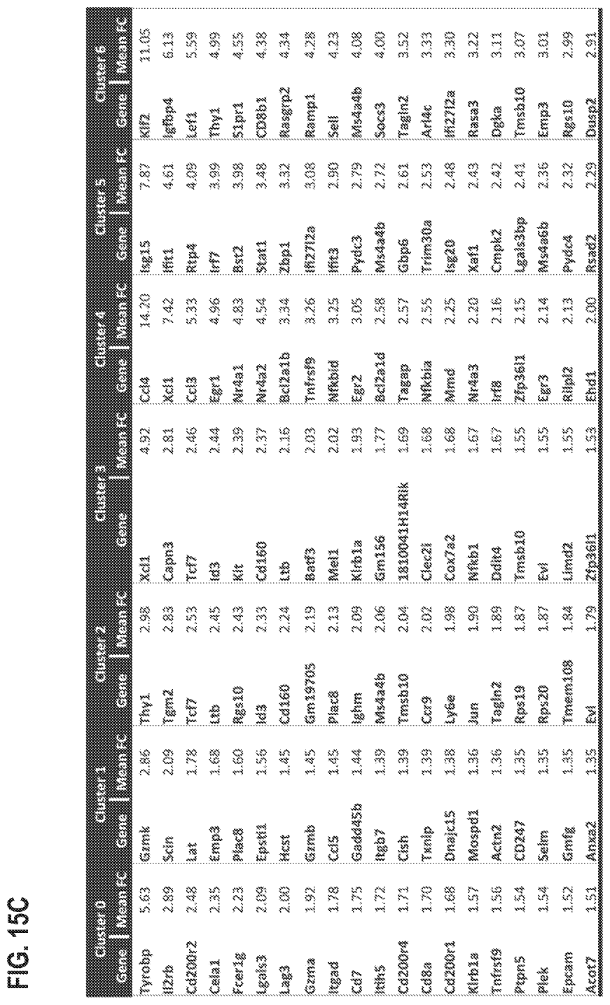

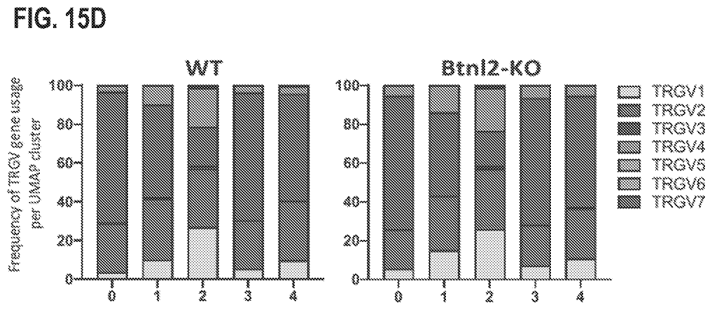

To further explore the relationship between TCR and γδ IEL transcriptome, scRNA-seq was performed on the same duodenal, jejunal, and ileal γδ IELs that were profiled for TCR sequencing. Nine clusters in each sample were identified (FIG. 15A), and the transcriptome of single γδ IELs in clusters 0, 1, and 3 differentiated between duodenal and ileal origin with jejunal γδ IELs exhibiting intermediate transcriptome profiles (FIGS. 15A-B).

Using the top 20 markers detected in each single cell cluster, in conjunction with molecular signatures described in recent scRNA-seq and bulk RNAseq reports (Mayassi et al., Cell, 2019. 176 (5): 967-981.e19; Crinier et al., Immunity, 2018. 49 (5): 971-986.e5.), γδ IEL attributes were proposed, such as differentiation stage, maturation, and effector profile, to distinguish among γδ IEL clusters (FIG. 15C). Clusters 0 and 1 contain mature and highly cytolytic IELs, clusters 2 and 3 include immature IELs, whereas cluster 4 consists of newly activated IELs undergoing transcriptional changes such as antigen-mediated differentiation (FIG. 15C). The remaining IELs were subdivided into smaller clusters with specialized effector profile such as type I/III interferon responses in cluster 5 (Isg15, Irf7, Stat1) and subset-specific differentiation stage such as recently emigrated CD8β⁺ IEL progenitors in cluster 6 (Klf2, Thy1, S1pr1, CD8b1, Sell) (FIG. 15C). With respect to TRGV distribution, TRGV7 gene usage was dominant in clusters 0-4, while ileal Btnl2-KO γδ IELs had reduced frequencies of TRGV7 and higher frequencies of TRGV1 and TRGV4 in cluster 1 compared to their WT counterparts (40.4% vs. 45.9%, 15.2% vs. 10.3%, and 13.1% vs. 9.5%, respectively; FIG. 15D).

Next, the distribution of CDR3γ/δ pairings was examined using the most common ileal CDR3γ, encompassing ˜10% of total CDR3γ clones across different segments and genotypes (Vγ7-J1, CASWAGYSSGFHKVF (SEQ ID NO: 29)). It was found that the top CDR3Y chain was preferentially enriched in cluster 0 of ileal Bin/2-KO γδ IELs (51.3% vs. 37.2%), which is defined by the largest number of maturation and cytolytic molecules (FIG. 14C), and dominated cluster 1 in WT γδ IELs (47.4% vs. 31.5%) (FIG. 6C). Pairing of the top γ chain bearing the same nucleotide sequence with distinct CDR3δ sequences shaped the transcriptome of the pairs, as they preferentially associated with specific clusters (FIG. 6D).

Collectively, these RNA-seq and scTCR-seq observations indicate that Btnl2 deficiency alters the transcriptome as well as the TRGV/TRDV repertoire of ileal γδ IELs, such that their antigenic specificities and antibacterial responses are changed. This disclosure is the first to describe intestinal γδ IEL transcriptome and TCR repertoire diversity simultaneously at single cell resolution, revealing a previously uncharacterized heterogeneity in duodenal, jejunal, and ileal γδ IELs that may account for compartment-specific immune responses driven by tissue-specific expression of immune-modulatory molecules.

### Example 9

### Btnl2-KO Mice Exhibit More Severe Intestinal Inflammation in Chronic DSS-Induced Colitis

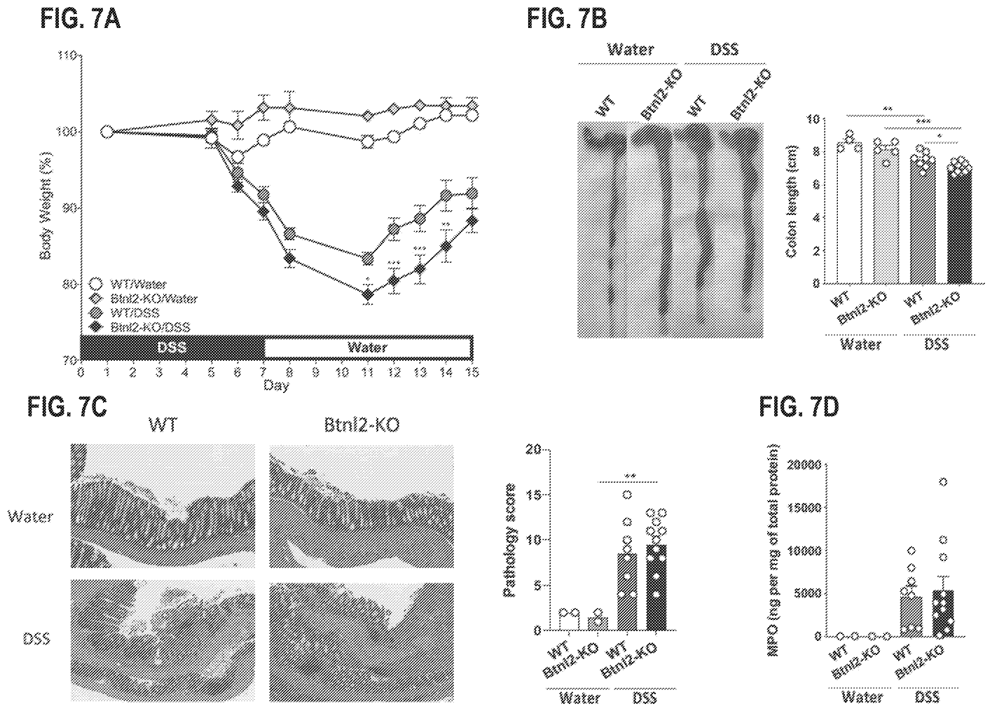

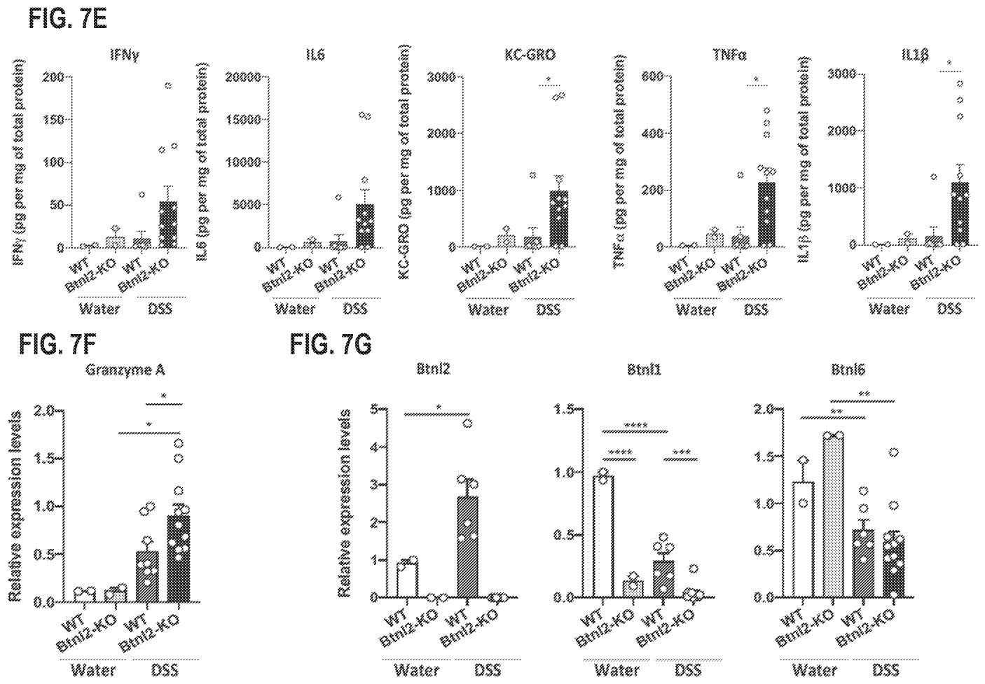

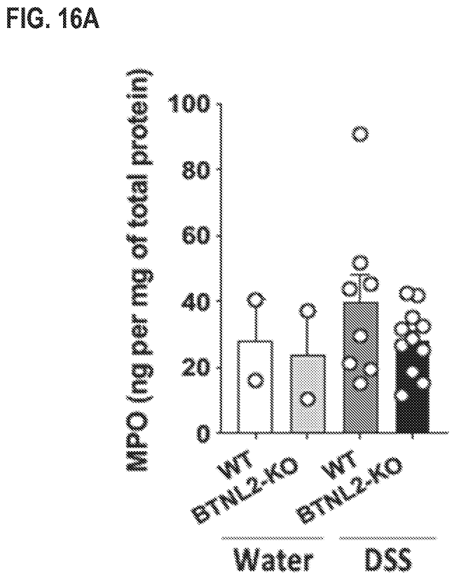

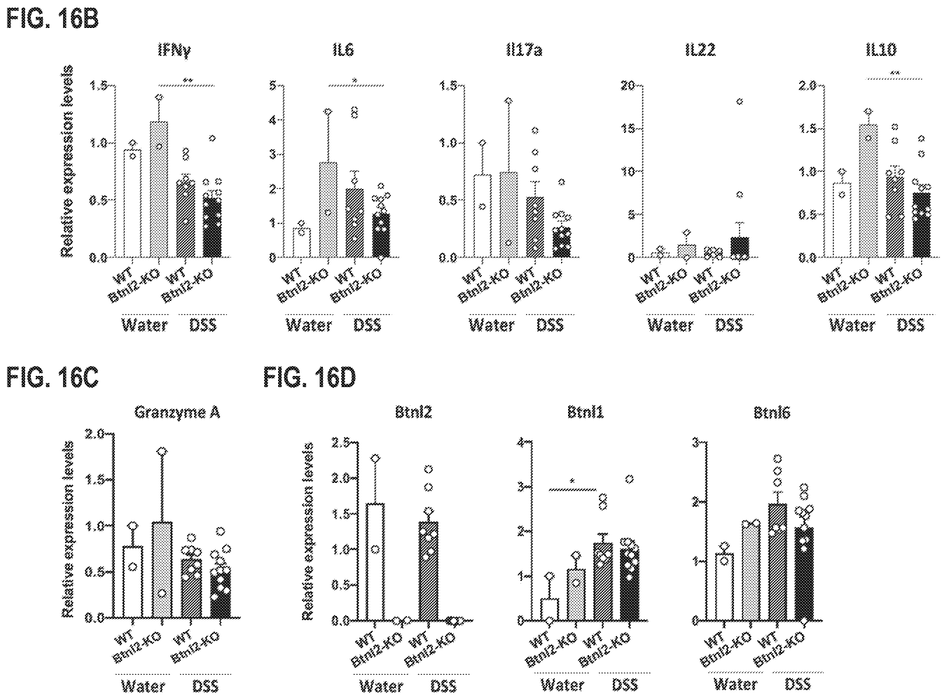

Btnl2 expression is reduced in the colon compared to the small intestine (FIG. 1C). However, significant upregulation in colonic Btnl2 levels was observed in colitic WT mice. Next, the impact of its deficiency was assessed on mucosal immune responses in the setting of DSS-induced epithelial injury. Cohoused Btnl2-KO and WT littermates were subjected to DSS-induced colitis by administering DSS for 7 days followed by 8 days of water. While Btnl2-KO and WT mice exhibited comparable intestinal damage in the early phase of the disease (day 7), as demonstrated by comparable body weight loss and increased myeloperoxidase activity (MPO) levels, a biomarker of intestinal injury and neutrophilia (FIGS. 7A-D), it was observed that Btnl2-KO mice exhibited a significant delay in body weight recovery compared to WT littermates during the repair phase of colitis (FIG. 7A). The observed delay in recovery was accompanied by significantly shorter colons, increased granzyme A levels, greater histopathological damage and MPO activity in the colon compared to WT littermates (FIGS. 7B-D and 7F). Notably, DSS-treated Btnl2-KO mice had ˜2-fold higher levels of pro-inflammatory cytokines such as IFNγ, IL-6, KC-GRO, TNFα, and IL-1β in the colon (FIG. 7E). In contrast, MPO activity, pro-inflammatory cytokine and granzyme A levels were not significantly altered in the ileum of DSS-treated Btnl2-KO mice compared to WT littermates (FIGS. 16A-C). Corroborating these results, Btnl2 transcripts were increased in the colon of DSS-treated WT mice, whereas the levels of other family members, such as Btnl1 and Btnl6, were decreased with DSS treatment (FIG. 7G). Btnl2 transcripts were unchanged in the ileum of DSS-treated, indicating that Btnl2 expression in the colon may be induced as a feedback regulatory mechanism at the site of injury to attenuate DSS-triggered inflammation and facilitate the recovery process (FIG. 16D).

### Example 10

Emerging research places Btn/Btnl family of molecules at the heart of γδ T cell development. This disclosure sheds light on Btnl2 as a regulator of ileal γδ IEL maintenance. Specifically, Btnl2 acts as a coinhibitory ligand to an unidentified receptor(s) on γδ IELs and regulates both proliferation and segment-specific effector profiles of ileal γδ IELs under homeostatic conditions.

Through the segment-focused approach, a temporal and spatial window was observed, during which Btnl2 exerted its functions on intestinal γδ IELs. ScTCRseq revealed that Vγ7⁺ IELs dominated the small intestine of 11-wk-old Btnl2-KO and WT mice, indicating that their development was not affected. Although Btnl2 impacted γδ IEL proliferation preferentially in the ileum, its deficiency reverberated throughout distinct segments of the small intestine. Specifically, despite the ability of Btnl2 to suppress both duodenal and jejunal/ileal γδ IEL proliferation in vitro, this was confined in vivo only to ileal γδ IEL expansion. However, at the molecular level, Btnl2 deficiency led to an altered Vγ usage among Vγ7 IELs and similarly altered Vδ usage across all three segments of the small intestine, indicating overlapping as well as unique roles for Btnl2 across the distinct segments of the small intestine. This, in turn, was accompanied by dysregulated antibacterial module in ileal Btnl2-KO γδ IELs, which may be relevant for mucosal repair and clearance of segment-tropic pathogenic microbes.

Consistent with a region-specific effect, duodenal Btnl2-KO CDR3γ and CDR3γ/δ clonal repertoires were not markedly different from those identified in cohoused WT littermates underscoring the ileum as the predominant site of Btnl2-mediated regulation at steady-state. Importantly, the most abundant Vγ clones in the ileal compartment of individual Btnl2-KO mice included Vγ1⁺ and Vγ4⁺ clones, in contrast to Vγ7⁺ clones exclusively enriched in WT mice. Btnl2 may be important for co-regulating ligands (i.e., Btnl1, Btnl6) of Vγ7⁺ TCRs during early adulthood. In support of this dynamic remodeling of the γδ TCRs, Btnl2 deficiency led to different convergent recombination events, such that pairing of the most common Vγ7⁺ clonotype with distinct Vδ sequences defined the transcriptome profiles of ileal γδ IELs. It is possible that site-specific metabolite levels and/or antigenic pressure led to multiple independent in situ recombination events, indicating an adaptive behavior of γδ IELs towards local environmental antigens. Since ileal γδ IEL motility along the villi-crypt axis is strictly dependent on the presence of microbiota, an impaired antibacterial profile could also be a consequence of improper localization or ineffective surveillance of ileal Btnl2-KO γδ IELs. Conversely, loss of epithelia-expressed Btnl2 could lead to alterations in the local microbiome, which would then drive reshaping of the TCR repertoire and antibacterial response module of ileal Btnl2-KO γδ IELs. Further studies are required to understand how the reshaped Vγ-Vδ repertoire alongside the defective antibacterial response module may affect the susceptibility of Btnl2-KO mice to small intestinal infectious agents.

An acute reliance on Btnl expression at a predefined time has been proposed for both murine Vγ7⁺ IEL development and human Vγ4⁺/V81-IEL maintenance. Specifically, Btnl1 expression in adult Btnl1-KO mice could not rescue Vγ7⁺ development (Di Marco Barros et al., Cell, 2016. 167 (1): 203-218), whereas mucosal repair and Btnl8 expression restoration following adherence to a gluten-free diet could not reconstitute Vγ4⁺/Vδ1⁺ IEL subsets in patients with celiac disease. In light of these observations, it was speculated that Btnl molecules may regulate not only the selective expansion of tissue-specific Vγ chains in neonates but also their TCR specificities across distinct tissue compartments in young adults. As such, segment-biased γδ TCR specificities may be determined by choice of dimerization partners among Btnl molecules and their nuanced spatial and temporal expression in the intestine. While Btnl1 and Btnl6 jointly affect Vγ7 selection and maturation, there is no known binding partner for Btnl2. In addition, no other Btnl molecules were induced in the intestine to compensate for the loss of Btnl2, indicating that their expression patterns were not co-regulated, despite being encoded at the same locus.

Of the various family members, structurally, Btnl2 is unique in that it lacks the antigen-binding B30.2 domain shared by most of the Btn/Btnl superfamily members, indicating that the inhibitory effect of Btnl2 may depend on the signaling pathways triggered downstream of engagement of its putative receptor on γδ IELs. Btnl2 could either homodimerize or heterodimerize with other intestine-specific Btnls through IgC interactions independent of B30.2 domains. As a heterodimer, Btnl2 interacting partner may contribute to the B30.2-driven activation of the heterodimer and binding to the putative receptor, whereby Btnl2 ligation would induce the downstream inhibition of proliferation. Btnl1, Btnl4, and Btnl6 can be candidate binding partners of Btnl2 due to their similar intestinal expression. Hence, despite higher expression on duodenal IECs, Btnl2 may exert more profound inhibition on ileal γδ IELs due to increased regional expression of its binding partner on IECs and putative receptor on γδ IELs. As such, this region-specific interaction may be promoted by local soluble antigens like bacterial metabolites. Btnl2 could also function as a receptor antagonist prohibiting the binding of another Btnl heterodimer to γδ TCR and suppressing γδ IEL proliferation. Alternatively, as this suppression is only partial, Btnl2 may indirectly target certain Vγ TCR(s) or TCR specificities via regulating surface expression of Vγ ligands (i.e., Btnl6 for Vγ7 TCRs). Further studies are required to address whether Btnl2 can exert its inhibitory effects on γδ IELs across all intestinal compartments during segment-specific inflammation.

Previous studies showed that γδ T cell depletion induces greater colonic damage, reduced KGF secretion, increased IFNγ production by γδ T cells and decreased IEC proliferation during DSS-induced colitis, indicating that γδ IELs can promote mucosal repair following epithelial injury. Conversely, impaired IL-10 production by Tregs leads to uncontrolled γδ IEL proliferation and spontaneous colitis in Pdk I^{f/f};CD4^{cre} mice, supporting a proinflammatory role for γδ IELs in the colon. Btnl2 expression is upregulated in the distal colon during DSS-induced colitis, and Btnl2-KO colitic mice exhibit a delay in recovery during the mucosal repair phase of the disease, potentially due to γδ IEL-dependent and independent (i.e., Tregs, proinflammatory helper T cells) mechanisms to controlling the damage caused by epithelial injury. Interestingly, Btnl1 and Btnl6 transcripts were downregulated in Btnl2-KO colitic mice, confirming previous reports in which Btnl1/6 transcripts were shown to be significantly reduced in distal colon of Muc2-KO mice and BTNL8 expression was diminished or lost in colonic and duodenal biopsies of patients with UC and celiac disease, respectively. Hence, IECs upregulate Btnl2 expression in response to environmental stress factors to limit damage-induced expansion of γδ IELs and induce release of antibacterial molecules. As such, the findings support the idea that close interaction between γδ IELs and epithelium-specific Btnl molecules throughout the small and large intestines drives their proliferation and function in homeostatic and inflammatory settings.

In conclusion, this disclosure demonstrates a novel role for Btnl2 in regulating the expansion of ileal γδ IELs, sculpting of their Vγ and Vγ-Vδ TCR specificities and altering their antibacterial response module. The scRNAseq and scTCRseq surveys revealed a highly dynamic γδ IEL compartment finely adapted to environmental cues of each segment of the small intestine during adulthood.

### Example 11

### Role of Btnl2 in Immune Function During Homeostasis and Disease

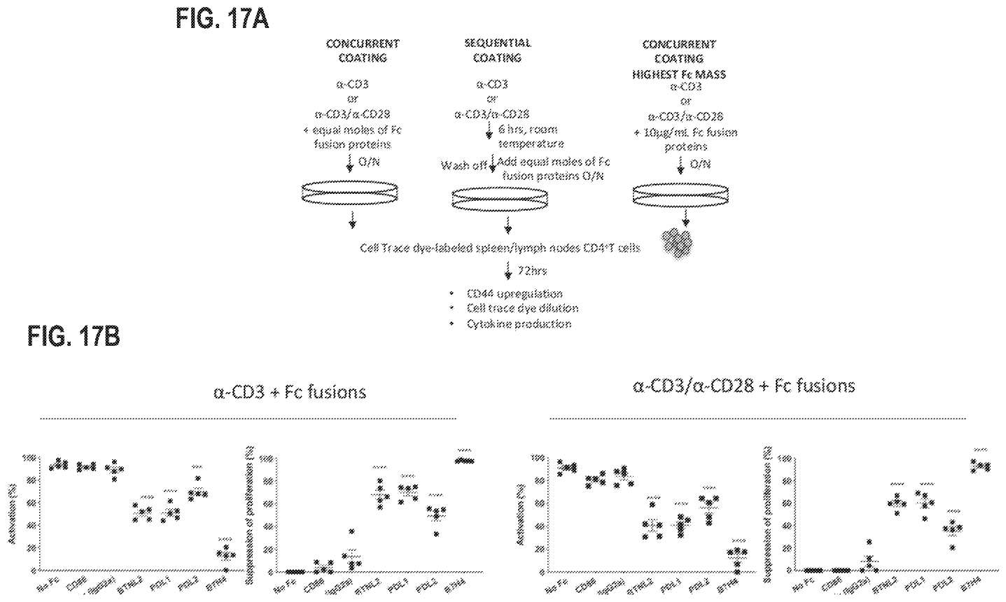

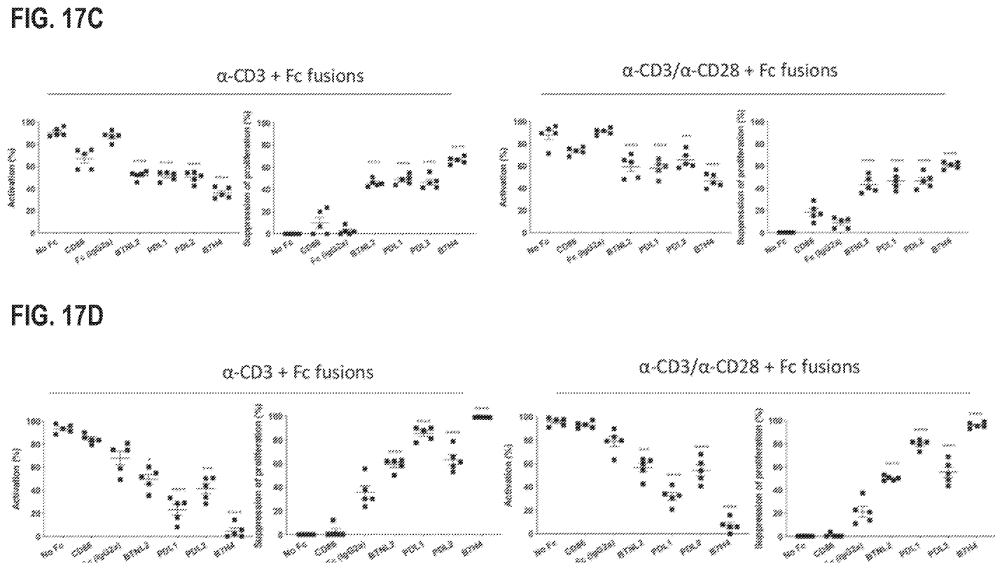

To understand how Btnl2 modulates CD4⁺ T cell function, the in vitro proliferation assay described above was performed. It was confirmed that recombinant Btnl2 inhibited both activation, proliferation, and cytokine secretion of CD4⁺T cells (FIGS. 11A-C). Addition of CD28 signal partially rescued Btnl2-mediated inhibition of proliferation, which also translated into partial rescue of cytokine production (e.g., TNFα, IL-6, and IFNγ), with IL-2 still at significantly lower levels in the presence of recombinant Btnl2 (FIGS. 11β-C). Recombinant Btnl2 inhibited CD4⁺ T cell proliferation similarly to PDL1, during sequential coating of α-CD3, α-CD28, and Fc fusion proteins or concurrent coating with equal mass rather than equal moles of Fc fusions (FIGS. 17A-D). These results indicate that Btnl2-driven inhibition of proliferation is a consequence of Btnl2 ligation with its putative receptor on activated CD4⁺T cells (FIG. 6D) rather than displacement of plate-bound α-CD3/α-CD28.

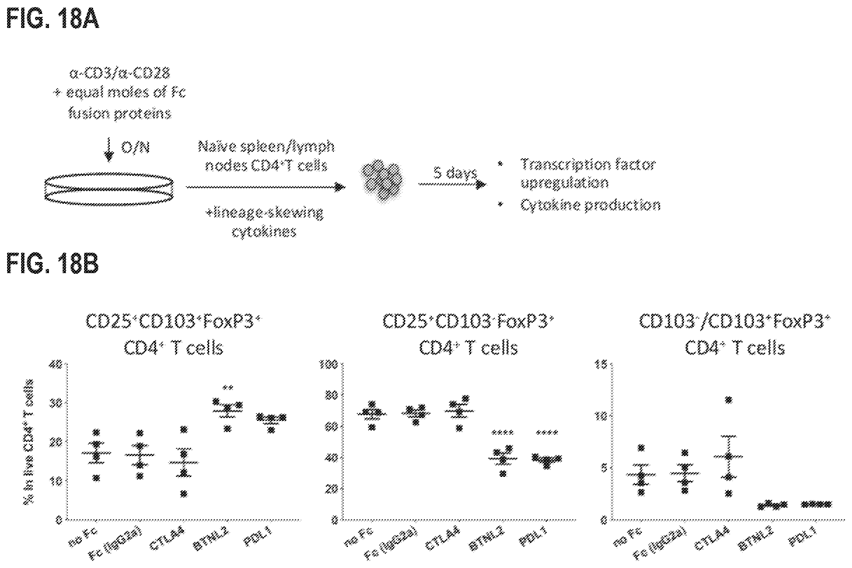

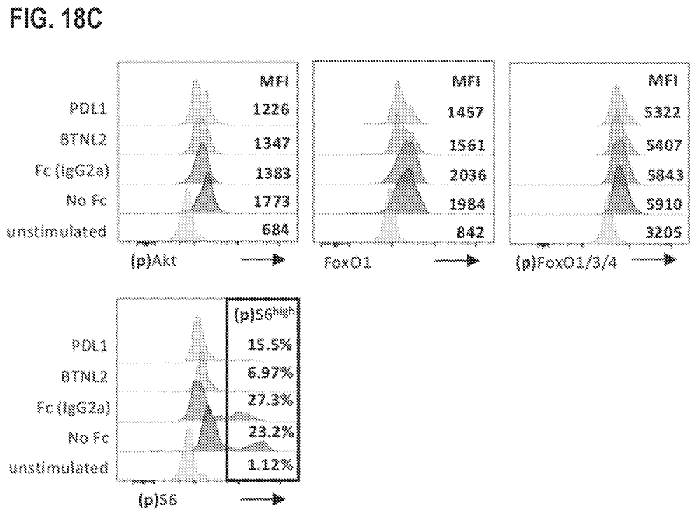

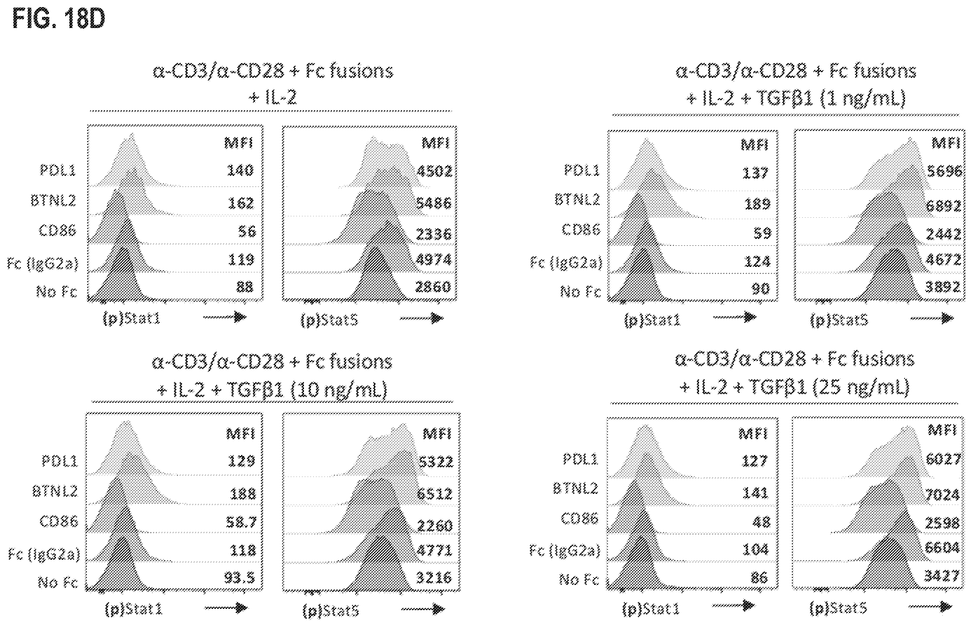

Next, whether Btnl2 affected T helper differentiation in the presence of lineage-specific cytokines was examined (FIG. 18A). It was found that recombinant Btnl2 could support de novo differentiation of tissue-resident-like, CD103⁺ regulatory T cells in the presence of IL-2 and TGFβ1 in vitro similarly to PDL1, bringing the ratio of CD103⁺/CD103⁺ to almost 1 (FIG. 18B). Low FoxO1 expression levels distinguish activated (non-lymphoid tissue-resident) from resting (spleen, lymph node) regulatory T cells. In the presence of recombinant Btnl2, regulatory T cells showed reduced Akt and S6 phosphorylation and FoxO1 expression, indicating that Btnl2 could promote differentiation of activated regulatory T cells (FIG. 18C). In addition, recombinant Btnl2 drove STAT5 phosphorylation in a TGFβ1 dose-dependent manner (FIG. 18D). Overall, these results indicated that Btnl2 preferentially induced tissue-resident-like regulatory T cells similarly to PDL1 in vitro.

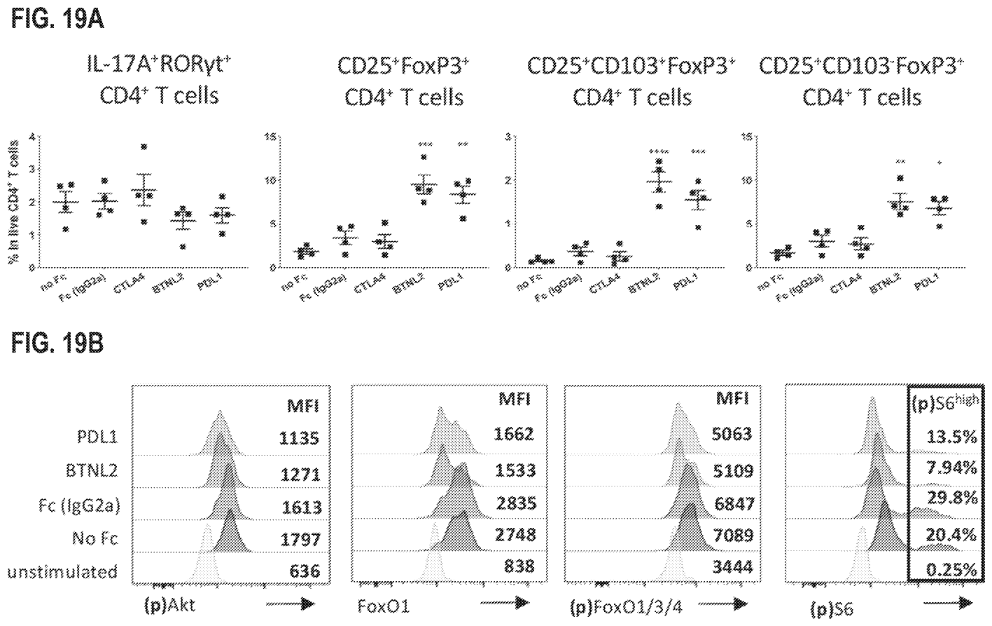

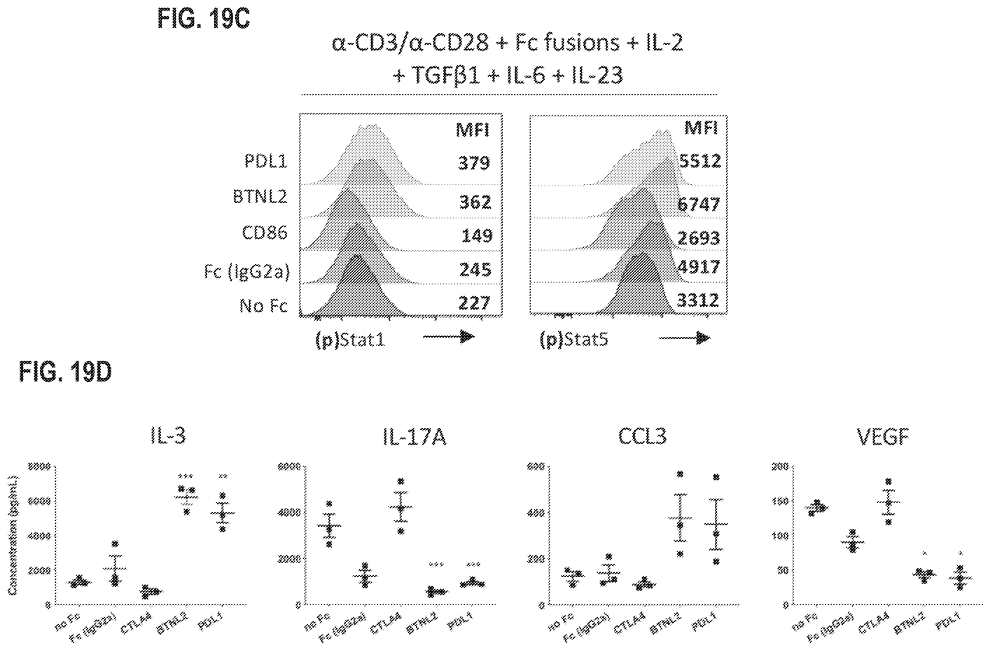

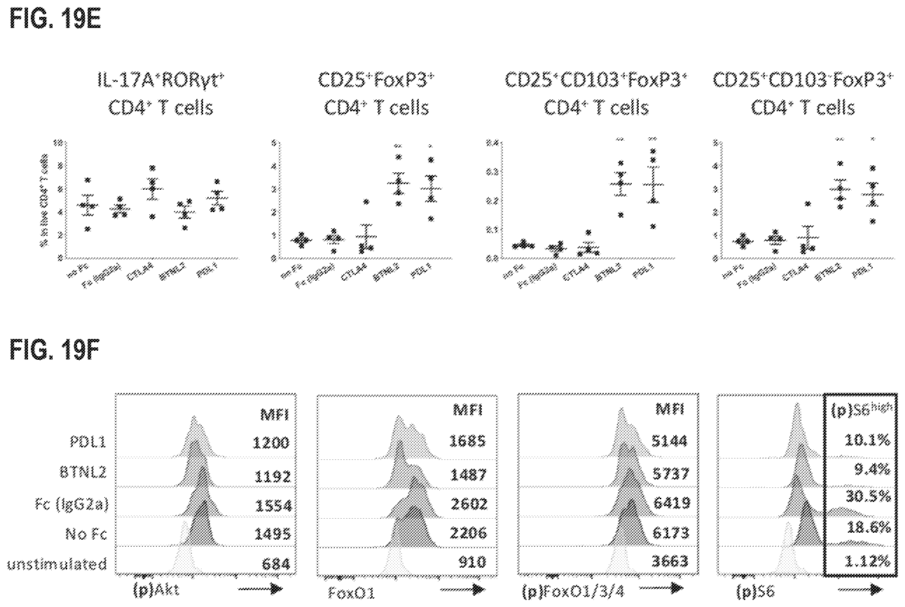

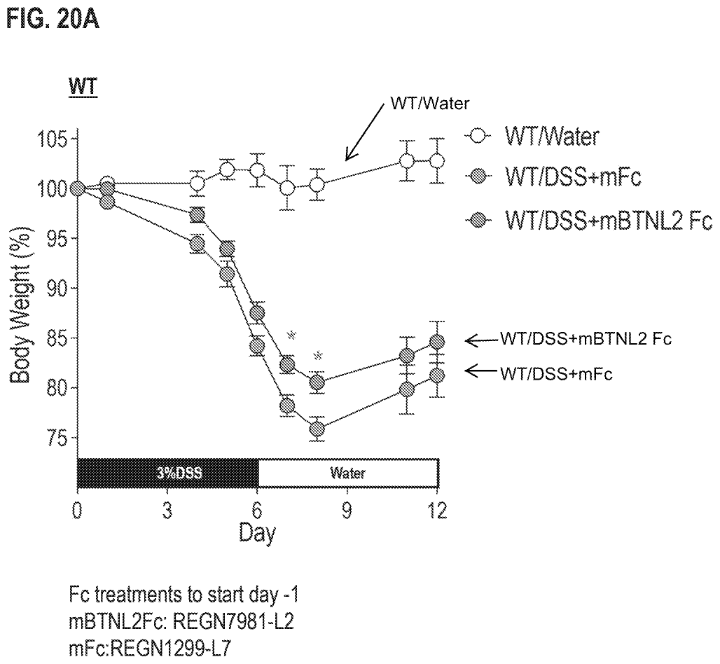

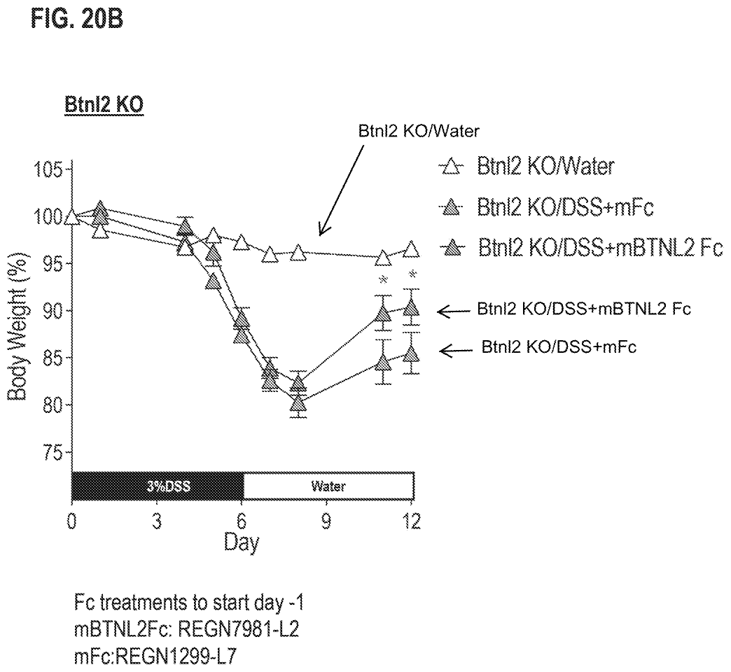

Within non-lymphoid tissues, a fine balance between pro-inflammatory T helper lineages such as Th1, Th2, and Th17 cells and tolerogenic regulatory T cells ensures context-appropriate immune responses. Given the plasticity of Th17 and regulatory T cell lineages and their interconnected developmental pathways, whether Btnl2 affected Th17 cell differentiation was examined. As shown in FIG. 19A, recombinant Btnl2 significantly drove the Th17 cell differentiation towards FoxP3⁺ regulatory T cell induction, similarly to PDL1, even in the presence of Th17-skewing cytokines in vitro. Notably, recombinant Btnl2 promoted CD103 FoxP3⁺ regulatory T cell differentiation (FIG. 19A) through similar mechanisms used in regulatory T cell-skewing conditions such as downregulation of Akt and S6 phosphorylation, reduction in FoxO1 expression levels (FIG. 19B), and upregulation of STAT5 phosphorylation (FIG. 19C).

In addition, levels of proinflammatory cytokines such as IL17A and VEGF were reduced, while CCL3 and IL3 levels were significantly increased in the presence of Btnl2 and PDL1 during Th17 cell-inducing conditions, indicating a shift towards regulatory T cell phenotype (FIG. 19D). By varying one cytokine at a time within the Th17 cell-inducing cytokine cocktail, it was shown that Btnl2-mediated skewing towards CD103 FoxP3⁺ regulatory T cells was strictly dependent on TGFβ1 levels, was enhanced by blockade of IL-6 signaling, occurred independently of IL-23, and was strongly inhibited by IL-1β (FIG. 19E). Specifically, IL17A RORγt⁺ Th17 cell frequencies were comparable across all Fc fusion conditions, and Btnl2-driven CD103⁺ FoxP3⁺ regulatory T cell induction was reduced 10-fold with the addition of IL-1β to the Th17 lineage-skewing cocktail of TGFβ1, IL-6, and IL-23 (FIG. 19E). Furthermore, IL-1β can inhibit the later stages of Btnl2-driven regulatory T cell induction since it did not rescue Akt and S6 phosphorylation and FoxO1 expression (FIG. 19F). Corroborating these results, Btnl2 can override the conventional Th17 program (e.g., TGFβ1, IL-6, and IL-23) towards tissue-resident-like regulatory T cells similarly to PDL1 in vitro.

### Example 12

BTNL2 Putative Loss-of-Function (pLoF) Variant Associates with Increased Odds of Inflammatory Bowel Disease

A genome wide association study was carried out on a genomic sequencing cohort comprising 14,274 cases and 439,809 controls, identifying a significant association of a rare pLoF variant (p.Glu454*/rs28362675) with an increased risk of IBD. Controlling the data for known common risk variants in the HLA class I or class II regions that are proximate to Btnl2 locus revealed that the association of Btnl2 Glu454* with IBD is independent of nearby IBD-associated HLA variants in Btnl2 (Table 3). FineMap analysis of the GWAS confirmed that p.Glu454*/rs28362675 is independent of other HLA signals in the region.

TABLE 3
A rare loss-of-function variant in Btnl2
(p.Glu454*; rs28362675) significantly associates with increased odds of IBD

| | Case | Control | OR | | |
|---|---|---|---|---|---|
| Variant | RR:RA:AA | RR:RA:AA | (95% CI) | p-value | AAF |
| 6:32394744:C:A | 14005:267:2 | 435160:4631:18 | 1.56 | 4.14E−8 | 0.0054 |
| | | | (1.33, 1.83) | | |

### Example 13

Effects of Treatments with a Btnl2-Fc Fusion Protein in WT and Btnl2 KO Mice with DSS-induced Colitis.

To induce chronic DSS-induced colitis, 15-week old female Btnl2 KO and WT mice with an average of more than 23 g were given 3% DSS (Sigma-Aldrich) in drinking water for 6 days, followed by water for additional 6 days. Control group received distilled water for the duration of the study. mBTNL2-Fc and mFc as control were administered intraperitoneally at 25 mg/kg biweekly starting on day −1. Mice were weighed and monitored for clinical signs of colitis (e.g., stool consistency and fecal blood) on a daily basis. Body weight loss was calculated as the percent difference between the original and actual body weight on any particular day. All bar charts represent means±SEM. Statistical significance between mBTNL2-Fc and mFc-treated groups was determined by two-way analysis of variance (ANOVA) with Tukey's multiple comparison post test (*p<0.05, ** p<0.005, *** p<0.001, **** p<0.0001). Data analysis was performed using GraphPad Prism software. As a result, prophylactic treatment with mBTNL2-Fc promoted epithelial barrier repair from DSS-induced mucosal damage and reduced the disease severity in WT and Btnl2 KO mice as reflected in increased body weight gain compared to the mFc control treatment group. Importantly, mBtnl2-Fc treatment was able to potently override any developmental changes associated with Btnl2 deficiency in mice, indicating that Btnl2 agent administration might be beneficial in improving colitis in patients carrying Btnl2 gene mutations.

### Example 14

Effects of Btnl2 Agent in Mouse Model of Multiple Sclerosis (Experimental Autoimmune Encephalomyelitis (EAE)) and Mouse Model of graft-versus-host Disease (GvHD)

The goal of this study is to establish whether treatment with a Btnl2 agent can alleviate symptoms and halt disease progression in a T cell-driven mouse model of multiple sclerosis such as EAE induced by the myelin oligodendrocyte glycoprotein (MOG) peptide comprising amino acids 35-55. As demonstrated in this disclosure (see, for example, EXAMPLE 4), Btnl2-Fc inhibits T cell proliferation and activation in vitro. Since EAE relies on high infiltration of activated T cells in central nervous system tissues such as brain and spinal cord, MOG35-55-induced EAE is likely a good disease model to test the role of this gene in regulating EAE severity.

10-12 week-old WT mice are immunized with a suboptimal dose of MOG35-55 in complete Freund's adjuvant (CFA) (50 μg/mouse compared to 200 μg/mouse in regular dosing) to induce EAE without saturating the system. Using a suboptimal dose of MOG35-55 allows a comparison of EAE induction and progression in the presence or absence of BTNL2-Fc. Four groups of 10 WT mice are immunized, each with CFA only (1 group) or MOG35-55 in CFA (3 groups).

Prior to the onset of paralysis in MOG35-55 groups on day 7-8 post-immunization, one group (no Fc fusion) is administered PBS, one group (isotype control) is administered negative control-Fc fusion, and one group (experimental Fc) is administered Btnl2-Fc fusion, twice per week for 3 weeks. We expect to observe no EAE in the CFA only group and to notice regular onset and disease progression of EAE in the MOG35-55 groups with no Fc fusion or with isotype control. We expect to observe a delay in EAE onset, a milder disease course and a quicker recovery in MOG35-55 group with BTNL2-Fc.

Next, at the onset of paralysis in MOG35-55 groups on day 10-14 post-immunization, one group (no Fc fusion) is administered PBS, one group (isotype control) is administered negative control-Fc fusion, and one group (experimental Fc) is administered Btnl2-Fc fusion, twice per week for 3 weeks. We expect to observe no EAE in the CFA only group and to notice regular onset and disease progression of EAE in the MOG35-55 groups with no Fc fusion or with isotype control. We expect to observe a delay in EAE peak of disease, a milder disease course and a quicker recovery in MOG35-55 group with BTNL2-Fc.

Another example is the mouse model of graft-versus-host disease (GvHD) with the goal to determine whether in vivo administration of Btnl2 agent can inhibit T cell proliferation, activation of Th1/Th17 cells and increase the generation of regulatory T cells in vivo, and thus prevent GvHD development. Briefly, bone marrow (BM) suspension is harvested from C57BL/6 mice by flushing the marrow from the femurs and tibias, and transplanted into letally irradiated BALB/c mice. mBtnl2-Fc fusion proteins are administered intravenously on day 0 and intraperitoneally on day 2. The control group receives mFc injections. The severity of GvHD is evaluated with a clinical GvHD scoring system. Mice are euthanized 14 days post-transplantation, and GvHD target organs such as liver and gut are harvested for histopathological, cytokine and flow cytometry analysis. We expect to observe increased survival and reduced gut GvHD associated with reduced T cell proliferation, Th1/Th17 cytokines and increased frequency of regulatory T cells with Btnl2-Fc treatment.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method of treating a subject having an inflammatory disorder, comprising:
(a) selecting a subject with an inflammatory disorder and an elevated level of intraepithelial lymphocytes (IELs) as compared to a predetermined reference value; and
(b) administering to the subject a therapeutically effective amount of a butyrophilin-like 2 (Btnl2) agent that is capable of decreasing proliferation of IELs in the subject by increasing a level or activity of Btnl2 in the subject, thereby treating the inflammatory disorder.

2. The method of claim 1, wherein the subject has at least one loss-of-function single nucleic polymorphism (SNP) at rs28362675 in the Btnl2 gene.

3. (canceled)

4. The method of claim 1, wherein the IELs comprise γδ T cells or intestinal γδ IELs.

5. (canceled)

6. The method of claim 4, wherein the intestinal γδ IELs comprise jejunal γδ IELs, ileal γδ IELs, colonic γδ IELs, or combinations thereof.

7. (canceled)

8. The method of claim 6, wherein the intestinal γδ IELs comprise ileal CD8αα* γδ IELs.

9. The method of claim 1, wherein the inflammatory disorder is selected from the group consisting of: an intestinal inflammatory disorder, an immune-mediated disease, an autoimmune disease, and a gut-associated immune-mediated disease.

10-12. (canceled)

13. The method of claim 9, wherein the immune-mediated disease comprises graft versus host disease (GVHD), Celiac disease, ulcerative colitis, Crohn's disease, rheumatoid arthritis, sarcoidosis, myositis, inflammatory bowel disease, gastrointestinal inflammation, or type I diabetes.

14. The method of claim 1, wherein the Btnl2 agent comprises a Btnl2 protein or variant thereof or a fusion protein comprising the Btnl2 protein or variant thereof.

15. The method of claim 14, wherein the Btnl2 protein comprises an amino acid sequence having at least 90% identity to an amino acid sequence of any one of SEQ ID NOs: 1-3 or comprises an amino acid sequence of any one of SEQ ID NOs: 1-3.

16. (canceled)

17. The method of claim 14, wherein the fusion protein comprises a MADCAM1 inhibitor.

18. The method of claim 17, wherein the MADCAM1 inhibitor is an anti-MADCAM1 antibody.

19. The method of claim 14, wherein the fusion protein comprises a Btnl2 protein or variant thereof and an anti-MADCAM1 antibody.

20. (canceled)

21. The method of claim 1, wherein the Btnl2 agent comprises a nucleic acid having a polynucleotide sequence encoding a Btnl2 protein or variant thereof or encoding a fusion protein comprising the Btnl2 protein or variant thereof.

22. (canceled)

23. The method of claim 1, wherein the subject is human.

24. The method of claim 1, wherein the Btnl2 agent is administered intratumorally, intravenously, subcutaneously, intraosseously, orally, transdermally, as a suppository, or sublingually.

25. The method of claim 1, wherein the Btnl2 agent is administered in a sustained release, controlled release, or delayed release dosage form.

26. The method of claim 1, further comprising administering to the subject an anti-inflammation agent.

27. (canceled)

28. A method of treating a subject having an inflammatory bowel disease (IBD), comprising:
(a) identifying a subject having an IBD and at least one loss-of-function SNP at rs28362675 in the Btnl2 gene; and
(b) administering to the subject a therapeutically effective amount of a Btnl2 agent capable of increasing a level or activity of Btnl2 in the subject, thereby treating the IBD.

29. The method of claim 28, wherein the subject has at least one loss-of-function SNP at rs28362675 that results in a Glu454Ter (stop codon) mutation.

30. The method of claim 28, wherein the Btnl2 agent comprises a Btnl2 protein or variant thereof or a fusion protein comprising the Btnl2 protein or variant thereof.

31. The method of claim 30, wherein the Btnl2 protein comprises an amino acid sequence having at least 90% identity to an amino acid sequence of any one of SEQ ID NOs: 1-3 or comprises an amino acid sequence of any one of SEQ ID NOs: 1-3.

32. The method of claim 28, wherein the Btnl2 agent comprises a nucleic acid having a polynucleotide sequence encoding a Btnl2 protein or variant thereof or a fusion protein comprising the Btnl2 protein or variant thereof.

33. (canceled)

34. (canceled)

35. A method of decreasing the number of IELs in a subject, comprising administering an effective amount of a Btnl2 agent to a subject in need thereof, the Btnl2 agent capable of increasing a level or activity of Btnl2 in the subject.

36. The method of claim 35, wherein the IELs comprise γδ T cells.

37. The method of claim 35, wherein the IELs comprise intestinal γδ IELs.

38. The method of claim 37, wherein the intestinal γδ IELs comprise jejunal γδ IELs, ileal γδ IELs, colonic γδ IELs, or combinations thereof.

39. (canceled)

40. The method of claim 38, wherein the intestinal γδ IELs comprise ileal CD8αα⁺ γδ IELs.

41. The method of claim 38, wherein the Btnl2 agent comprises a Btnl2 protein or variant thereof or a fusion protein comprising the Btnl2 protein or variant thereof.

42. The method of claim 41, wherein the Btnl2 protein comprises an amino acid sequence having at least 90% identity to an amino acid sequence of any one of SEQ ID NOs: 1-3 or comprises an amino acid sequence of any one of SEQ ID NOs: 1-3.

43. (canceled)

44. The method of claim 41, wherein the fusion protein comprises a MADCAM1 inhibitor.

45. The method of claim 44, wherein the MADCAM1 inhibitor is an anti-MADCAM1 antibody.

46. (canceled)

47. The method of claim 35, wherein the Btnl2 agent comprises a nucleic acid having a polynucleotide sequence encoding a Btnl2 protein or variant thereof or encoding a fusion protein comprising the Btnl2 protein or variant thereof.

48. (canceled)

49. A method of eliciting an antimicrobial response in a subject with an elevated level of intraepithelial lymphocytes (IELs) as compared to a predetermined reference value, comprising administering to the subject an effective amount of a Btnl2 agent, the Btnl2 agent capable of increasing a level or activity of Btnl2 in the subject.

50. The method of claim 49, wherein the IELs comprise γδ T cells.

51. The method of claim 49, wherein the IELs comprise intestinal γδ intraepithelial lymphocytes (IELs).

52. The method of claim 51, wherein the intestinal γδ IELs comprise jejunal γδ IELS, ileal γδ IELs, colonic γδ IELs, or combinations thereof.

53. (canceled)

54. The method of claim 52, wherein the intestinal γδ IELs comprise ileal CD8αα⁺ γδ IELs.

55. The method of claim 49, wherein the Btnl2 agent comprises a Btnl2 protein or variant thereof or a fusion protein comprising the Btnl2 protein or variant thereof.

56. The method of claim 55, wherein the Btnl2 protein comprises an amino acid sequence having at least 90% identity to an amino acid sequence of any one of SEQ ID NOs: 1-3 or comprises an amino acid sequence of any one of SEQ ID NOs: 1-3.

57. (canceled)

58. The method of claim 55, wherein the fusion protein comprises a MADCAM1 inhibitor.

59. The method of claim 58, wherein the MADCAM1 inhibitor is an anti-MADCAM1 antibody.

60. (canceled)

61. The method of claim 49, wherein the Btnl2 agent comprises a nucleic acid having a polynucleotide sequence encoding a Btnl2 protein or variant thereof or encoding a fusion protein comprising the Btnl2 protein or variant thereof.

62. (canceled)

63. A method for identifying a Btnl2 agent capable of decreasing IELs in a subject, comprising:
(a) administering to the subject an amount of a Btnl2 agent capable of increasing a level or activity of Btnl2 or having Btnl2 agonist activity;
(b) performing an assay on a sample obtained from the subject and determining the number of the IELs in the sample; and
(c) identifying the Btnl2 agent as having capability of decreasing the IELs in the subject if the subject has an increased number of IELs as compared to a predetermined reference value.

64-66. (canceled)


SEQUENCE LISTING (60 sequences)

SEQ ID NO: 1 - protein, 482 aa, synthetic construct
  REGION 1..482: note = Synthetic
MVDFPGYNLSGAVASFLFILLTMKQSEDFRVIGPAHPILAGVGEDALLTCQLLPKRTTMHVEVRWYRSEPSTPVFVHRDGVEVTEMQMEEYRGWVEWIENGIAKGNVALKIHNIQPSDNGQYWCHFQDGNYCGETSLLLKVAGLGSAPSIHMEGPGESGVQLVCTARGWFPEPQVYWEDIRGEKLLAVSEHRIQDKDGLFYAEATLVVRNASAESVSCLVHNPVLTEEKGSVISLPEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVSLGSSPLITVEGQEDGEMQPMCSSDGWFPQPHVPWRDMEGKTIPSSSQALTQGSHGLFHVQTLLRVTNISAVDVTCSISIPFLGEEKIATFSLSESRMTFLWKTLLVWGLLLAVAVGLPRKRS

SEQ ID NO: 2 - protein, 366 aa, synthetic construct
  REGION 1..366: note = Synthetic
MVDFPGYNLSGAVASFLFILLTMKQSGLGSAPSIHMEGPGESGVQLVCTARGWFPEPQVYWEDIRGEKLLAVSEHRIQDKDGLFYAEATLVVRNASAESVSCLVHNPVLTEEKGSVISLPEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVSLGSSPLITVEGQEDGEMQPMCSSDGWFPQPHVPWRDMEGKTIPSSSQALTQGSHGLFHVQTLLRVTNISAVDVTCSISIPFLGEEKIATFSLSESRMTFLWKTLLVWGLLLAVAVGLPRKRS

SEQ ID NO: 3 - protein, 272 aa, synthetic construct
  REGION 1..272: note = Synthetic
MVDFPGYNLSGAVASFLFILLTMKQSEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVSLGSSPLITVEGQEDGEMQPMCSSDGWFPQPHVPWRDMEGKTIPSSSQALTQGSHGLFHVQTLLRVTNISAVDVTCSISIPFLGEEKIATFSLSESRMTFLWKTLLVWGLLLAVAVGLPRKRS

SEQ ID NO: 4 - DNA, 1449 bp, synthetic construct
  misc_feature 1..1449: note = Synthetic
  source 1..1449: mol_type = other DNA
atggtggattttccaggctacaatctgtctggtgcagtcgcctccttcctattcatcctgctgacaatgaagcagtcagaagactttagagtcattggccctgctcatcctatcctggccggggttggggaagatgccctgttaacctgccagctactccccaagaggaccacaatgcacgtggaggtgaggtggtaccgctcagagcccagcacacctgtgtttgtgcacagggatggagtggaggtgactgagatgcagatggaggagtacagaggctgggtagagtggatagagaatggcattgcaaagggaaatgtggcactgaagatacacaacatccagccctccgacaatggacaatactggtgccatttccaggatgggaactactgtggagaaacaagcttgctgctcaaagtagcaggtctggggtctgcccctagcatccacatggagggacctggggagagtggagtccagcttgtgtgcactgcaaggggctggttcccagagccccaggtgtattgggaagacatccggggagagaagctgctggccgtgtctgagcatcgcatccaagataaagatggcctgttctatgcggaagccaccctggtggtcaggaacgcctctgcagagtctgtgtcctgcttggtccacaaccccgtcctcactgaggagaaggggtcggtcatcagcctcccagagaaactccagactgagctggcttctttaaaagtgaatggaccttcccagcccatcctcgtcagagtgggagaagatatacagctaacctgttacctgtcccccaaggcgaatgcacagagcatggaggtgaggtgggaccgatcccaccgttaccctgctgtgcatgtgtatatggatggggaccatgtggctggagagcagatggcagagtacagagggaggactgtactggtgagtgacgccattgacgagggcagactgaccctgcagatactcagtgccagaccttcggacgacgggcagtaccgctgcctttttgaaaaagatgatgtctaccaggaggccagtttggatctgaaggtggtaagtctgggttcttccccactgatcactgtggaggggcaagaagatggagaaatgcagccgatgtgctcttcagatgggtggttcccacagccccacgtgccatggagggacatggaaggaaagacgataccatcatcttcccaggccctgactcaaggcagccacgggctgttccacgtgcagacattgctaagggtcacaaacatctccgctgtggacgtcacttgttccatcagcatcccctttttgggcgaggagaaaatcgcaactttttctctctcagagtccaggatgacgtttttgtggaaaacactgcttgtttggggattgcttcttgctgtggctgtaggcctgcccaggaagaggagctga

SEQ ID NO: 5 - DNA, 1215 bp, synthetic construct
  misc_feature 1..1215: note = Synthetic
  source 1..1215: mol_type = other DNA
ccgggtgtgtcacagcctcacttaggcacagcgtttccttgtctttgttccagggaggatggtggattttccaggctacaatctgtctggtgcagtcgcctccttcctattcatcctgctgacaatgaagcagtcagtctggggtctgcccctagcatccacatggagggacctggggagagtggagtccagcttgtgtgcactgcaaggggctggttcccagagccccaggtgtattgggaagacatccggggagagaagctgctggccgtgtctgagcatcgcatccaagataaagatggcctgttctatgcggaagccaccctggtggtcaggaacgcctctgcagagtctgtgtcctgcttggtccacaaccccgtcctcactgaggagaaggggtcggtcatcagcctcccagagaaactccagactgagctggcttctttaaaagtgaatggaccttcccagcccatcctcgtcagagtgggagaagatatacagctaacctgttacctgtcccccaaggcgaatgcacagagcatggaggtgaggtgggaccgatcccaccgttaccctgctgtgcatgtgtatatggatggggaccatgtggctggagagcagatggcagagtacagagggaggactgtactggtgagtgacgccattgacgagggcagactgaccctgcagatactcagtgccagaccttcggacgacgggcagtaccgctgcctttttgaaaaagatgatgtctaccaggaggccagtttggatctgaaggtggtaagtctgggttcttccccactgatcactgtggaggggcaagaagatggagaaatgcagccgatgtgctcttcagatgggtggttcccacagccccacgtgccatggagggacatggaaggaaagacgataccatcatcttcccaggccctgactcaaggcagccacgggctgttccacgtgcagacattgctaagggtcacaaacatctccgctgtggacgtcacttgttccatcagcatcccctttttgggcgaggagaaaatcgcaactttttctctctcagagtccaggatgacgtttttgtggaaaacactgcttgtttggggattgcttcttgctgtggctgtaggcctgcccaggaagaggagctgaaaagagtgaatgtgacattggcttcaaacacagctcacctgagactgatttcttctg

SEQ ID NO: 6 - DNA, 934 bp, synthetic construct
  misc_feature 1..934: note = Synthetic
  source 1..934: mol_type = other DNA
ccgggtgtgtcacagcctcacttaggcacagcgtttccttgtctttgttccagggaggatggtggattttccaggctacaatctgtctggtgcagtcgcctccttcctattcatcctgctgacaatgaagcagtcagagaaactccagactgagctggcttctttaaaagtgaatggaccttcccagcccatcctcgtcagagtgggagaagatatacagctaacctgttacctgtcccccaaggcgaatgcacagagcatggaggtgaggtgggaccgatcccaccgttaccctgctgtgcatgtgtatatggatggggaccatgtggctggagagcagatggcagagtacagagggaggactgtactggtgagtgacgccattgacgagggcagactgaccctgcagatactcagtgccagaccttcggacgacgggcagtaccgctgcctttttgaaaaagatgatgtctaccaggaggccagtttggatctgaaggtggtaagtctgggttcttccccactgatcactgtggaggggcaagaagatggagaaatgcagccgatgtgctcttcagatgggtggttcccacagccccacgtgccatggagggacatggaaggaaagacgataccatcatcttcccaggccctgactcaaggcagccacgggctgttccacgtgcagacattgctaagggtcacaaacatctccgctgtggacgtcacttgttccatcagcatcccctttttgggcgaggagaaaatcgcaactttttctctctcagagtccaggatgacgtttttgtggaaaacactgcttgtttggggattgcttcttgctgtggctgtaggcctgcccaggaagaggagctgaaaagagtgaatgtgacattggcttcaaacacagctcacctgagactgatttcttctg

SEQ ID NO: 7 - DNA, 20 bp, synthetic construct
  misc_feature 1..20: note = Synthetic
  source 1..20: mol_type = other DNA
ggattgcccacggtatagtc

SEQ ID NO: 8 - DNA, 19 bp, synthetic construct
  misc_feature 1..19: note = Synthetic
  source 1..19: mol_type = other DNA
aggaccgaccactctgaag

SEQ ID NO: 9 - DNA, 22 bp, synthetic construct
  misc_feature 1..22: note = Synthetic
  source 1..22: mol_type = other DNA
tatctggcgtggctgcctcctt

SEQ ID NO: 10 - DNA, 21 bp, synthetic construct
  misc_feature 1..21: note = Synthetic
  source 1..21: mol_type = other DNA
ggtgcagatgccggaatacag

SEQ ID NO: 11 - DNA, 21 bp, synthetic construct
  misc_feature 1..21: note = Synthetic
  source 1..21: mol_type = other DNA
gccacacttcccatgtcaatg

SEQ ID NO: 12 - DNA, 24 bp, synthetic construct
  misc_feature 1..24: note = Synthetic
  source 1..24: mol_type = other DNA
caggacccagatggtgagacaagc

SEQ ID NO: 13 - DNA, 20 bp, synthetic construct
  misc_feature 1..20: note = Synthetic
  source 1..20: mol_type = other DNA
gaggccatcttggaactgaa

SEQ ID NO: 14 - DNA, 20 bp, synthetic construct
  misc_feature 1..20: note = Synthetic
  source 1..20: mol_type = other DNA
ccaccgtcttctggaccttt

SEQ ID NO: 15 - DNA, 21 bp, synthetic construct
  misc_feature 1..21: note = Synthetic
  source 1..21: mol_type = other DNA
tggcagcaatgggctctgtcc

SEQ ID NO: 16 - DNA, 23 bp, synthetic construct
  misc_feature 1..23: note = Synthetic
  source 1..23: mol_type = other DNA
gggaagccgaacatactgaactg

SEQ ID NO: 17 - DNA, 24 bp, synthetic construct
  misc_feature 1..24: note = Synthetic
  source 1..24: mol_type = other DNA
cccgttcttcagcatttggatttc

SEQ ID NO: 18 - DNA, 23 bp, synthetic construct
  misc_feature 1..23: note = Synthetic
  source 1..23: mol_type = other DNA
acgtaacacagttccacccgcct

SEQ ID NO: 19 - DNA, 23 bp, synthetic construct
  misc_feature 1..23: note = Synthetic
  source 1..23: mol_type = other DNA
ggcgctttgattgaaaagaactg

SEQ ID NO: 20 - DNA, 24 bp, synthetic construct
  misc_feature 1..24: note = Synthetic
  source 1..24: mol_type = other DNA
tgttctggctccttattgattgag

SEQ ID NO: 21 - DNA, 24 bp, synthetic construct
  misc_feature 1..24: note = Synthetic
  source 1..24: mol_type = other DNA
tgactgctgcccactgtaacgtgg

SEQ ID NO: 22 - DNA, 22 bp, synthetic construct
  misc_feature 1..22: note = Synthetic
  source 1..22: mol_type = other DNA
acactctttccctacacgacgc

SEQ ID NO: 23 - DNA, 25 bp, synthetic construct
  misc_feature 1..25: note = Synthetic
  source 1..25: mol_type = other DNA
ttcctgggagtccaggatrgtattg

SEQ ID NO: 24 - DNA, 28 bp, synthetic construct
  misc_feature 1..28: note = Synthetic
  source 1..28: mol_type = other DNA
cacccttatgacttcaggaaagaacttt

SEQ ID NO: 25 - DNA, 27 bp, synthetic construct
  misc_feature 1..27: note = Synthetic
  source 1..27: mol_type = other DNA
ttccacaatcttcttggatgatctgag

SEQ ID NO: 26 - DNA, 22 bp, synthetic construct
  misc_feature 1..22: note = Synthetic
  source 1..22: mol_type = other DNA
acactctttccctacacgacgc

SEQ ID NO: 27 - DNA, 57 bp, synthetic construct
  misc_feature 1..57: note = Synthetic
  source 1..57: mol_type = other DNA
gtgactggagttcagacgtgtgctcttccgatctgtcccagycttatggagatttgt

SEQ ID NO: 28 - DNA, 61 bp, synthetic construct
  misc_feature 1..61: note = Synthetic
  source 1..61: mol_type = other DNA
gtgactggagttcagacgtgtgctcttccgatcttagtcacctctttagggtagaaatctt

SEQ ID NO: 29 - protein, 15 aa, synthetic construct
  REGION 1..15: note = Synthetic
CASWAGYSSGFHKVF

SEQ ID NO: 30 - protein, 14 aa, synthetic construct
  REGION 1..14: note = Synthetic
CSYGLYSSGFHKVF

SEQ ID NO: 31 - protein, 14 aa, synthetic construct
  REGION 1..14: note = Synthetic
CASWAYSSGFHKVF

SEQ ID NO: 32 - protein, 14 aa, synthetic construct
  REGION 1..14: note = Synthetic
CASWGYSSGFHKVF

SEQ ID NO: 33 - protein, 15 aa, synthetic construct
  REGION 1..15: note = Synthetic
CAVWMRYSSGFHKVF

SEQ ID NO: 34 - protein, 13 aa, synthetic construct
  REGION 1..13: note = Synthetic
CAVWIGTSWVKIF

SEQ ID NO: 35 - protein, 15 aa, synthetic construct
  REGION 1..15: note = Synthetic
CASWAGRSSGFHKVF

SEQ ID NO: 36 - protein, 15 aa, synthetic construct
  REGION 1..15: note = Synthetic
CASWAVYSSGFHKVF

SEQ ID NO: 37 - protein, 15 aa, synthetic construct
  REGION 1..15: note = Synthetic
CASWAPYSSGFHKVF

SEQ ID NO: 38 - protein, 14 aa, synthetic construct
  REGION 1..14: note = Synthetic
CASWVYSSGFHKVF

SEQ ID NO: 39 - protein, 14 aa, synthetic construct
  REGION 1..14: note = Synthetic
CASWAGSSGFHKVF

SEQ ID NO: 40 - protein, 14 aa, synthetic construct
  REGION 1..14: note = Synthetic
CASWADSSGFHKVF

SEQ ID NO: 41 - protein, 15 aa, synthetic construct
  REGION 1..15: note = Synthetic
CASWAGGSSGFHKVF

SEQ ID NO: 42 - protein, 16 aa, synthetic construct
  REGION 1..16: note = Synthetic
CASWAGKYSSGFHKVF

SEQ ID NO: 43 - protein, 15 aa, synthetic construct
  REGION 1..15: note = Synthetic
CASWAEYSSGFHKVF

SEQ ID NO: 44 - protein, 14 aa, synthetic construct
  REGION 1..14: note = Synthetic
CAVWVYSSGFHKVF

SEQ ID NO: 45 - protein, 14 aa, synthetic construct
  REGION 1..14: note = Synthetic
CAVWGYSSGFHKVF

SEQ ID NO: 46 - protein, 13 aa, synthetic construct
  REGION 1..13: note = Synthetic
CAVWNSSGFHKVF

SEQ ID NO: 47 - protein, 15 aa, synthetic construct
  REGION 1..15: note = Synthetic
CASWAGDSSGFHKVF

SEQ ID NO: 48 - protein, 15 aa, synthetic construct
  REGION 1..15: note = Synthetic
CASWALYSSGFHKVF

SEQ ID NO: 49 - protein, 14 aa, synthetic construct
  REGION 1..14: note = Synthetic
CASWEYSSGFHKVF

SEQ ID NO: 50 - protein, 11 aa, synthetic construct
  REGION 1..11: note = Synthetic
CASSSGFHKVF

SEQ ID NO: 51 - protein, 15 aa, synthetic construct
  REGION 1..15: note = Synthetic
CASWAGHSSGFHKVF

SEQ ID NO: 52 - protein, 15 aa, synthetic construct
  REGION 1..15: note = Synthetic
CAVWMRYSGFHKVFF

SEQ ID NO: 53 - protein, 19 aa, synthetic construct
  REGION 1..19: note = Synthetic
CALMERGISEGYSTDKLVF

SEQ ID NO: 54 - protein, 21 aa, synthetic construct
  REGION 1..21: note = Synthetic
CALWEPIWHIGGIRATDKLVF

SEQ ID NO: 55 - protein, 16 aa, synthetic construct
  REGION 1..16: note = Synthetic
CALMERIGLTTDKLVF

SEQ ID NO: 56 - protein, 17 aa, synthetic construct
  REGION 1..17: note = Synthetic
AMGTLSEGYELGDKLVF

SEQ ID NO: 57 - protein, 22 aa, synthetic construct
  REGION 1..22: note = Synthetic
CASGFLWHLYRRDTSPTDKLVF

SEQ ID NO: 58 - protein, 17 aa, synthetic construct
  REGION 1..17: note = Synthetic
CALMELSEGPATDKLVF

SEQ ID NO: 59 - protein, 21 aa, synthetic construct
  REGION 1..21: note = Synthetic
CASGYTVWQIGGIRATDKLVF

SEQ ID NO: 60 - protein, 21 aa, synthetic construct
  REGION 1..21: note = Synthetic
CALSEPSAVDRRDTKTDKLVF